(19) United States
(12) Patent Application Publication
(10) Pub. No.: US 2022/0003777 A1
(43) Pub. Date: Jan. 6, 2022

(54) Methods Employing Mucin-Specific Proteases

(71) Applicant: The Board of Trustees of the Leland Stanford Junior University, Stanford, CA (US)

(72) Inventors: Carolyn R. Bertozzi, Menlo Park, CA (US); Stacy A. Malaker, Menlo Park, CA (US); Kayvon Pedram, Stanford, CA (US); Dayeon Shon, Stanford, CA (US)

(21) Appl. No.: 17/291,376

(22) PCT Filed: Nov. 7, 2019
(86) PCT No.: PCT/US2019/060346

§ 371 Date: May 5, 2021

Related U.S. Application Data
(60) Provisional application No. 62/757,585, filed on Nov. 8, 2018.

(51) Int. Cl. G01N 33/68; G01N 33/574

(52) U.S. Cl. CPC G01N 33/6848; G01N 33/6818; G01N 33/574; G01N 2333/988; G01N 2333/4725; G01N 2333/95; G01N 2400/00; C12Y 402/02001

(57) ABSTRACT

The present disclosure provides compositions and methods involving the use of mucin-specific proteases for mucin-specific cleavage, labeling, and/or enrichment of mucin domain glycoproteins. Also provided are methods for the analysis of mucin-domain glycoproteins useful in glycomapping of mucin glycosites and their associated glycoforms. Provided compositions and methods are also useful for selective cleavage, release, and enrichment of mucins from cell and tissue samples, for the study of native mucin biology, and for the detection and analysis of mucins that are aberrantly expressed in various conditions, including cancer.

### CROSS REFERENCE TO PRIORITY APPLICATION

This application claims the benefit of U.S. Provisional Application No. 62/757,585, filed Nov. 8, 2018, which is incorporated herein by reference in its entirety.

### STATEMENT OF GOVERNMENT SUPPORT

This invention was made with Government support under contract GM059907 awarded by the National Institutes of Health. The Government has certain rights in the invention.

### INTRODUCTION

Mucins are a class of proteins whose closely-spaced serine- and threonine-bound glycans (O-glycans) enforce a rigid extended structure. In addition to being a major structural component of native mucus, which coats all wet epithelial surfaces in the body and serves as the first line of defense against pathogens, mucins are found on cell surfaces on nearly every cell of the human body, where their towering structures act as physical barriers, glycocalyx stiffening agents, receptor ligands, and mediators of intracellular signaling.

Aberrant mucin expression and glycosylation are reliable biomarkers of carcinomas in humans Indeed, the membrane-associated mucin MUC1 is aberrantly expressed in ˜60% of all cancers diagnosed each year in the U.S. (Jonckheere et al. Biochimie (2010) 92, 1-11), rendering MUC1 one of the most prominently dysregulated genes in cancer. Another mucin, MUC16 (also called CA125), is highly expressed in ovarian cancer and clinically used as a biomarker for treatment efficacy and surveillance. The functional roles of not only the C-terminal mucin signaling domain, but also the heavily glycosylated mucin ectodomain, in promoting tumor progression have also been identified. For example, the MUC1 ectodomain alone can drive tumor progression by enhancing cancer cell survival and promoting proliferation in the metastatic niche. In addition, mucin-based vaccines, small molecule and antibody therapies, and chimeric antigen receptor (CAR)-T cell therapies have been and are being developed.

### SUMMARY

Provided herein are compositions, methods and kits involving the selective cleavage of mucin-domain glycoproteins using a mucin-specific protease (i.e., “mucinase”). Also provided are methods of analysis that employ selective cleavage of mucin-domain glycoproteins using a mucinase. The specificity of the mucinase for mucins derives from its recognition of a mucin-specific glycan-peptide cleavage motif, which involves a combination of peptide and glycan motifs within the mucin domain. Treatment of biological samples with the mucinase results in cleavage of the peptide backbone of the mucin-domain glycoprotein upon recognition of the mucin-specific glycan-peptide cleavage motif by the mucinase. Such cleavage releases glycosylated peptide fragments (i.e., glycopeptides) containing various glycans.

Released glycopeptides may be employed for subsequent glycomapping, obtaining of glycosignatures, and the like. Useful mucin-specific proteases (mucinases) include secreted protease of C1 esterase inhibitor (StcE), recombinant StcE polypeptides, including those comprising a sequence that has at least 90% sequence identity to SEQ ID NO:1. StcE variants and mutants may also find use in the subject compositions, methods, and kits. Also of interest are polynucleotides encoding StcE or variants or recombinants thereof, including e.g., nucleic acids comprising a sequence having at least 70% sequence identity to SEQ ID NO:2.

Additional mucinases are provided that may be employed for the selective cleavage of mucin-domain glycoproteins, staining of cells/tissues expressing mucin-domain glycoproteins, and for other methods, such as those as described herein. These mucinases include mucinases of serine peptidases family, e.g., Family S6, mucinases of zinc metallopeptidase family, e.g., Family M26, Family M60, or Family M66. These mucinases may have an amino acid sequence at least 90% identical (e.g., 91%, 92%, 93, 94%, 95%, 96%, 97%, 98%, 99%, or 100% identical) to a mucinase sequence provided in Table 1.

In some embodiments, glycosignatures may be produced and/or employed in the subject methods. Useful glycosignatures will vary and may include those produced by analyzing cleaved mucin-domain glycoproteins. In some instances, the selective analysis of mucin-domain glycoproteins and subsequent glycomapping provides glycosignatures. Glycosignatures produced in the subject methods may be employed for various purposes, including e.g., to facilitate the detection of disease conditions that are characterized by aberrant glycosylation and associated with particular glycosignatures.

Also provided are methods of identifying a receptor as mucin-domain glycoprotein-binding. Such methods may be performed for various purposes, including but not limited to e.g., identifying whether a receptor, such as an orphan receptor, binds a mucin-domain glycoprotein ligand.

Kits are also provided, including but not limited to where such kits may be employed in any of the methods described herein.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1C, illustrates that StcE is a protease that specifically cleaves mucins. (FIG. 1A) A “mucinase” would enable mucin-domain glycoproteins to be selectively removed from cells and tissue and cut into fragments, facilitating their analysis. (FIG. 1B) Recombinant or isolated glycoproteins were treated with recombinant StcE at a 1:10 enzyme:substrate (E:S) ratio for 3 h at 37° C. and the digests were separated by SDS-PAGE. Glycoproteins and glycosylated peptide fragments were visualized with periodate-based Emerald 300 Glycoprotein Stain® (Thermo Fisher Scientific). Corresponding silver stained gel images are shown below, in FIG. 9. (FIG. 1C) Recombinant human MUC16 was digested with StcE, E447D, or trypsin with and without prior enzymatic deglycosylation (Deglycosylation Mix, Promega). The digestion products were visualized as in (FIG. 1B).

FIG. 2A-2E, shows that StcE exhibits peptide-, glycan-, and secondary structure-based specificity for mucins. (FIG. 2A) The glycoproteins shown in FIG. 1B were digested with StcE. The digest was then deglycosylated by treatment with PNGase F, trypsinized, and analyzed by MS. Sequences of the StcE-dependent cleavage products were used as WebLogo inputs (weblogo.berkeley.edu). StcE recognizes the consensus sequence S/T*-X-S/T, and cleaves the peptide backbone before the P1'S/T only when the P2 S/T is glycosylated (indicated with an asterisk). Detected glycoforms on P2 are shown. Parenthesis indicate that the linkage for the second sialic acid of the disialylated structure could not be assigned. (FIG. 2B) Examples of StcE-cleaved N-terminal peptides from several recombinant mucins, with assigned glycan structures shown. (FIG. 2C) StcE, E447D, and trypsin were reacted with a native peptide backbone N-carboxyanhydride (NCA) derived co-polymer consisting of 50% GalNAc-α-O-serine and 50% lysine, with and without prior enzymatic deglycosylation (Delgycosylation Mix, Promega). Arrow indicates the StcE band. (FIG. 2D) StcE was incubated with RPPIT*QSSL (SEQ ID NO:3) at an E:S ratio of 1:10 for 3 h at 37° C. and subjected to MS analysis. Electron transfer dissociation (ETD) spectrum is shown. (FIG. 2E) Structure of StcE and the model peptide Ac-P(GalNAcα-)TL(GalNAcα-)TH-NMe (SEQ ID NO:4) following docking using the Molecular Operating Environment (MOE) software suite and the X-ray crystal structure of StcE (PDB ID: 3UJZ).

FIG. 3A-3B, illustrates that StcE increased the number of assigned glycosites, number of localized glycans, and sequence coverage of every protein studied. (FIG. 3A) Recombinant substrates were digested with StcE, de-N-glycosylated with PNGaseF, trypsinized, then subjected to MS using a higher energy collision-induced dissociation (HCD)-triggered electron transfer dissociation (ETD) instrument method. ETD spectra were used to assign glycosites. (FIG. 3B) ETD spectra of N- and C-terminal StcE-cleaved peptides LSTMMSPTT (SEQ ID NO:5) and STNASTVPFR (SEQ ID NO:6) (top) from CD43 from the experiment described in (FIG. 3A). ETD spectrum from the control sample (PNGaseF and trypsin only) is shown in the bottom panel. Lowercase ‘n’ denotes deamidation. Parentheses indicate that the sites modified with GalNAc residues could not be assigned.

FIG. 4A-4F, illustrates that StcE can cleave native mucins from cancer patient-derived ascites fluid and cultured cell surfaces. (FIG. 4A) StcE was incubated at a 1:10 E:S ratio for 3 h at 37° C. with a semi-crude patient-derived commercial preparation of MUC16 (Lee BioSolutions). Anti-MUC16 Western blot is shown, as MUC16 was a minority of the material by total protein stain (see FIG. 14 for silver stain). The MUC16 (Abcam, X75) antibody binds to extracellular repeat domains. (FIG. 4B) Crude ovarian cancer patient-derived ascites fluid was incubated with StcE for 1 h at 37° C. at the concentrations shown. (FIG. 4C) SKBR3 cells were treated with 50 nM StcE for 2 h at 37° C., then subjected to live cell flow cytometry with staining for MUC16 and HER2. (FIG. 4D) Western blot of MCF10A cells expressing signaling deficient MUC1 (MUC1ΔCT) on a doxycycline (dox) promoter. StcE treatment was performed on live cells as in (FIG. 4C). The MUC1 antibody (Cell Signaling Technology, VU4H5) binds to extracellular repeat domains. (FIG. 4E) BT-20, HeLa, and K562 cells were treated with StcE as in (FIG. 4C) and subjected to live cell flow cytometry with staining for MUC1 or MUC16. (FIG. 4F) Plated HeLa cells incubated in Hank's Buffered Salt Solution (HBSS) were treated with StcE at the times and concentrations shown. Supernatants were lyophilized, resuspended in sample buffer, separated by SDS-PAGE, and immunoblotted for MUC16.

FIG. 5A-5F, shows that Siglec-7 binds mucin-domain glycoproteins. (FIG. 5A) Siglecs are a family of leukocyte receptors that bind sialylated ligands of unknown identity. Similar to PD-1, upon ligand binding, they transmit inhibitory signals through intracellular ITSM and ITIM domains. (FIG. 5B) SKBR3 cells were treated with 50 nM StcE or E447D for 2 h at 37° C., stained with Siglec-7-Fc and Siglec-9-Fc, and subjected to live cell flow cytometry (top). Mean fluorescence intensity of three biological replicates is shown (bottom). Error bars are standard deviations. **=p<0.005 by Student's two-tailed t-test. (FIG. 5C) SKBR3 cells treated as in (FIG. 5B) were washed, stained with anti-His-FITC or Isotype-FITC, and subjected to live cell flow cytometry. (FIG. 5D) SKBR3 cells treated with 50 nM StcE, 50 nM E447D, or 30 nM Vibrio cholerae sialidase for 2 h at 37° C. were subjected to periodate-based sialic acid labeling followed by fixed cell flow cytometry. (FIG. 5E) Flow cytometry as in (b) on HeLa, ZR-75-1, BT-20, and MDA-MB-453 cells. (FIG. 5F) Flow cytometry analysis of ldlD CHO cells, with galactose (Gal) and GalNAc rescue conditions shown. Staining was performed with Siglec-7-Fc or Siglec-9-Fc.

FIG. 6 illustrates the expression of StcE and the inactive point mutant E447D. Detection was performed with Coomassie stain. Both StcE and E447D ran below the predicted molecular weight of 98 kDa. Purity of the enzymes was estimated at >90% by densitometry.

FIG. 7 illustrates StcE cleavage of C11NH, with controls. Detection was performed with periodate-based Emerald 300 Glycoprotein Stain® (Thermo Fisher Scientific). C11NH was treated with StcE or the inactive point mutant E447D at an enzyme to substrate ratio of 3:10 for the times shown. The reaction slowed significantly in the presence of 25 mM EDTA, consistent with StcE's zinc-dependent active site chemistry. E447D was not completely inactive (see lanes 7 and 8).

FIG. 8 illustrates that StcE is stable to lyophilization. The known StcE substrate C11NH was treated with StcE, E447D, lyophilized/resuspended StcE, or lyophilized/resuspended E447D for 15 and 120 min. Enzyme to substrate ratio was 1:10. Cleavage activity was not reduced after lyophilization. The arrow denotes the StcE band. Detection was with silver stain.

FIG. 9 illustrates silver staining of the gel shown in FIG. 1B. Arrow denotes the StcE band.

FIG. 10A-10D, illustrates optimization of StcE digest conditions. (FIG. 10A) Length of digestion. Trypsin, E447D, and StcE were reacted with 0.125 μg MUC16 in a total volume of 15 μl for 0.25, 1, 3, 6, and 12 h (the latter with and without the addition of EDTA). (FIG. 10B) Enzyme:substrate (E:S) ratio. Three conditions were tested: 1:20, 1:10, and 3:10 E:S. E447D and EDTA were added as negative controls. (FIG. 10C) E:S optimization with higher concentration. The second reaction was repeated with 0.5 μg substrate in 15 The increased concentration aided StcE digestion. (FIG. 10D) Deglycosylation and trypsin optimization. For all proteins tested, trypsin cleaved the post-StcE cleavage glycopeptides into sizes amenable for MS analysis. Optimal conditions for gel screens were therefore: 0.5 μg substrate, 1:10 E:S ratio, total volume of 15 μL, buffer 0.1% Protease Max in 50 mM ammonium bicarbonate, 3 h at 37° C. For MS samples, substrate content in the cleavage reactions was increased to 1-5 μg.

FIG. 11 illustrates that StcE can cleave S/T*-S/T. While the preferred amino acid sequence is S/T*-X-S/T, in the absence of this motif, StcE cleaved PSGL-1 sequences in which the amino acid spacer (X) was missing, meaning the cleavage motif S/T*-S/T is permitted. Treatment of PSGL-1 was performed as described in panel (a) of Figure.

FIG. 12 illustrates glycopeptide docking studies with a previously reported crystal structure of StcE (Yu et al., Structure (2012) 20, 707-717). The docked peptides Ac-PTLTH-NMe (magenta sticks; SEQ ID NO:7), Ac-P(GalNAcα-)TLTH-NMe (cyan sticks; SEQ ID NO:8), and Ac-P(GalNAcα-)TL(GalNAcα-)TH-NMe (green sticks; SEQ ID NO:4) derived from a StcE-labile peptide sequence in podocalyxin all adopted a common backbone conformation that was consistent with that of ligands bound to homologous metzincin enzymes (PDB IDs: 1QJI and 3VI1) (Gomis-Rüth et al., (2009) J. Biol. Chem. 284, 15353-15357).

FIG. 13 illustrates that StcE increased the total number of peptides with N-terminal T/S. Using the PNGaseF treated samples, the total number of peptides whose N-terminus was either serine or threonine (“TS peptides”) was calculated. In all proteins studied, the total number of TS peptides was higher due to the presence of StcE-cleaved peptides (“StcE-consensus peptides”). The increase in TS peptides may aid in database searches of StcE-cleaved samples.

FIG. 14 illustrates silver staining of a commercial semi-crude MUC16 preparation, along with positive (C11NH) and negative (fetuin) controls, corresponding to FIG. 4A and with identical treatment conditions. Arrow denotes the StcE band. MUC16 was a minority of the total protein present in the semi-crude preparation. Therefore, an anti-MUC16 Western blot was needed to detect MUC16 cleavage.

FIG. 15 illustrates uncut total protein (left) and anti-MUC16 (right) blots corresponding to FIG. 4B. The numbered lanes are those shown in FIG. 4B. The unnumbered band is 1000 nM E447D treatment for 1 h at 37° C. E447D was not completely inactive (see also FIG. 7).

FIG. 16A-16B, illustrates that StcE treatment was nontoxic to both adherent (HeLa) and suspension (K562) cell lines. (FIG. 16A) Cellular viability was read out using a resorufin-based dye (PrestoBlue, Thermo Fisher Scientific). Proliferation was unaffected over the course of 4 days in the presence of up to 500 nM StcE. (FIG. 16B) Live cell epifluorescent images of K562 (top) and HeLa (bottom) cells at 24 hours post treatment, with the same samples as those used in (a). Consistent with its status as a hemagglutinin, StcE treatment resulted in clumping of K562 cells. HeLa cells did not exhibit morphological changes.

FIG. 17 illustrates uncut blots corresponding to FIG. 4D, along with total protein and longer film exposure. The numbered lanes are those shown in FIG. 4D. The lanes directly to the left were from samples plated on a different growth substrate not relevant to the work presented here. Comparison of lanes 1 and 2 in the 30 s exposure reveals cleavage of MUC1 by StcE in the uninduced MCF10A cells.

FIG. 18 illustrates results from flow cytometry analysis corresponding to FIG. 4D. Doxycycline (dox) induction of MCF10A MUC1ΔCT cells resulted in an increase in cell surface anti-MUC1 antibody binding. StcE treatment at 50 nM for 2 h at 37° C. of both induced and uninduced cells resulted in a substantial loss of anti-MUC1 antibody binding.

FIG. 19A-19B illustrates results from Flow cytometry and western blot analysis of HeLa cells corresponding to supernatants shown in FIG. 4F. After StcE treatment at the times and concentrations shown, supernatants were removed and cells were (FIG. 19A) fixed and subjected to flow cytometry or (FIG. 19B) lysed and subjected to Western blotting. In both cases, a time and concentration dependent decrease in MUC16 positive signal was observed.

FIG. 20 illustrates that covalent conjugation of StcE to beads enables pull-down of the known StcE substrate C11NH from a 1:10 mixture of C11NH:BSA. StcE was conjugated to POROS AL beads (see Experimental Procedures). A portion of the beads (50 μL) was incubated with 10 μg BSA and 1 μg C11NH in a total volume of approx. 100 μL PBS. EDTA was added at 25 mM to inhibit cleavage of the substrate. After the reaction, the beads were pelleted and the supernatants were saved (“flow through”). The beads were washed once with 100 μL PBS (“wash 1”), once with 100 μL 1% Tween (“wash 2”), and once with 100 μL PBS (“wash 3”). For the elution, 32 μL 1×NuPage LDS sample buffer (Thermo Fisher Scientific) was added and the beads were boiled. Samples visualized by silver stain.

FIG. 21A-21C, illustrates that Siglec-7- and -9-Fc binding was sialidase sensitive, while only Siglec-7-Fc binding signal was StcE sensitive. (FIG. 21A) Treatment for 2 h at 37° C. with 30 nM Vibrio cholerae sialidase decreased Siglec-7- and -9-Fc binding on K562 cells, confirming that the observed signal was sialic acid dependent. Unstrained controls are shown in purple, and were analyzed with a higher laser power. (FIG. 21B) Sialidase treatment of ldlD CHO cells as in (FIG. 21A) also abrogated Sigec-7- and -9-Fc binding, as expected. (FIG. 21C) StcE treatment did not decrease Siglec-9-Fc binding in any ldlD CHO rescue condition. Siglec-7-Fc binding decreased by 4.1-fold with GalNAc only supplementation and 1.8-fold with Gal and GalNAc supplementation, but not in any condition where GalNAc was omitted.

FIG. 22 depicts candidate mucinases grouped by peptidase family according to the MEROPS database.

FIG. 23 shows expression and purification of recombinant mucinases from Family M60. Detection was performed with Coomassie stain.

FIG. 24 shows expression and purification of recombinant mucinases from Family M26, Family M66, and Family S6. Detection was performed with Coomassie stain.

FIG. 25 shows that the mucinases exhibit unique activities against native mucins. Recombinant mucinases were incubated at a 1:1 enzyme:substrate (E:S) ratio with 0.5 μM human plasma-derived C1 esterase inhibitor (C11NH) for 21 h at 37° C. either with or without 10 nM Vibrio cholerae sialidase (VC Sia). Digests were separated by SDS-PAGE and glycosylated peptides were visualized with Pro-Q Emerald 300 Glycoprotein Stain® (Thermo Fisher Scientific). Each mucinase candidate produced a unique banding pattern of digest products and exhibited differences in sialic acid sensitivity.

FIG. 26 illustrates the recombinant expression and purification of the inactive point mutants AM0627 E326A and BT4244 E575A. AM0627 E326A and BT4244 E575A were purified via His affinity chromatography, with an additional size exclusion chromatography (SEC) step for BT4244 E575A. Protein bands were detected with Coomassie stain.

FIGS. 27A-27B illustrate a decrease in catalytic activity for StcE E447D, BT4244 E575A and AM0627 E326A compared to their active enzyme counterparts. (FIG. 27A) 1 μM C11NH was treated with the appropriate mucinases at an E:S ratio of 1:5 for 20 h at 37° C. The activities of the point mutants were compared to other forms of enzyme inactivation, including addition of 25 mM EDTA and heat inactivation (HI) at 65° C. for 10 minutes. Glycosylated fragments were visualized with Pro-Q Emerald 300 Glycoprotein Stain® (Thermo Fisher Scientific). (FIG. 27B) Point mutant mucinase activity was tested at high concentration (1 μM) and higher E:S ratio (1:2) against C11NH at 37° C. for 18 h with or without the addition of 10 nM Vibrio cholerae sialidase (VC Sia). Proteins were visualized with Coomassie stain. Even under harsher digest conditions, point mutant mucinases exhibited little to no catalytic activity.

FIG. 28 shows that Alexa Fluor 647-labeled StcE E447D (AF647-E447D) (degree of labeling: 4.39 mol dye/mol E447D) is capable of staining live cells. HeLa or K562 cells were treated with 50 nM mucinase for 2 h at 37° C., stained with 50 nM to 100 nM (5 μg/mL-10 μg/mL) AF647-E447D for 30 min at 4° C., and subjected to live cell flow cytometry. Fold-change in mean fluorescence intensity with respect to an untreated control (dotted line) is shown for n=2 or 3 biological replicates. Binding levels were sensitive to removal of mucins by mucinase treatment and blocking of sites with StcE E447D prior to staining.

FIG. 29 demonstrates that Alexa Fluor 647-labeled BT4244 E575A (AF647-E575A) (degree of labeling: 6.12 mol dye/mol E447D) is capable of staining live cells. K562 cells were treated with 50 nM mucinase for 2 h at 37° C., stained with 100 nM (10 μg/mL) AF647-E575A for 30 min at 4° C., and subjected to live cell flow cytometry. Fold-change in mean fluorescence intensity with respect to an untreated control (dotted line) is shown for n=2 or 3 biological replicates. E575A staining was most sensitive to pretreatment with its active counterpart (BT4244) compared to pretreatment with other mucinases, reflecting its more selective glycoepitope binding properties.

FIG. 30 shows that live cell staining with Alexa Fluor 647-labeled BT4244 E575A (AF647-E575A) increases with knockout of the COSMC chaperone and VC sialidase treatment. Wild-type K562 cells and COSMC knockout K562 cells were incubated with 10 nM VC sialidase for 2 h at 37° C., stained with 100 nM (10 μg/mL) AF647-E575A for 1 h at 4° C., and subjected to live cell flow cytometry (n=3 biological replicates). The increase in staining with VC sialidase pretreatment reflects the sensitivity of BT4244 E575A to terminal sialic acid residues. Knockout of the COSMC chaperone prevents elongation of mucin-type O-glycans beyond the initiating N-acetylgalactosamine. The highest staining was observed for sialidase-treated COSMC knockout cells, indicating the selectivity of BT4244 E575A for the Tn antigen.

FIG. 31 illustrates that StcE E447D is capable of selectively staining mucin-domain glycoproteins by Western blot. A serially diluted 1:1 mixture of C11NH and bovine serum albumin (BSA) was transferred to a 0.2 μm nitrocellulose membrane and incubated with 20 μg/mL StcE E447D overnight at 4° C. IRdye800CW-labeled ReadyTag anti-6-His (Bio X Cell) was used as a secondary. Total protein was visualized using REVERT stain (LI-COR Biosciences). The signal was selective for C11NH over the non-mucin BSA and was visible down to 0.03 μg C11NH.

FIGS. 32A-32B show that StcE E447D is capable of identifying StcE-sensitive proteins in cell lysates by Western blot. (FIG. 32A) Untreated and StcE-treated (100 nM StcE, 1.5 h, 37° C.) HeLa lysates were transferred to a 0.2 μm nitrocellulose membrane and incubated with anti-MUC16 antibody (Abcam, X75) or 10 μg/mL biotin-StcE E447D (1.89 mol biotin/mol E447D). MUC16 and additional StcE-sensitive proteins were visualized through StcE E447D binding. (FIG. 32B) Untreated and StcE-treated K562 lysates were transferred to a nitrocellulose membrane and incubated with anti-MUC1 antibody (EMD Millipore, 214D4) or 10 μg/mL biotin-StcE E447D. MUC1 and additional StcE-sensitive bands were visualized through StcE E447D binding. IRdye800CW-streptavidin (LI-COR Biosciences) was used as a secondary for E447D blots and for secondary-only control blots. IRdye800CW goat anti-mouse IgG (LI-COR Biosciences) was used as a secondary for MUC16 and MUC1 blots.

FIGS. 33A-33B demonstrate that StcE E447D is capable of selectively staining a panel of mucin-domain glycoproteins by Western blot while BT4244 E575A stains a subset of this panel. (FIG. 33A) 1 μg of each substrate was transferred to a 0.2 μm nitrocellulose membrane and incubated with 5 μg/mL biotin-StcE E447D (1.89 mol biotin/mol E447D). (FIG. 33B) 1 μg of each substrate was treated with 10 nM VC sialidase for 1 h at 37° C., transferred to a 0.2 μm nitrocellulose membrane, and incubated with 5 μg/mL biotin-BT4244 E575A (1.37 mol biotin/mol E575A). IRdye800CW-streptavidin (LI-COR Biosciences) was used as a secondary. Total protein was visualized using REVERT stain (LI-COR Biosciences).

FIG. 34 depicts mucinase consensus motifs determined for ZmpC, BT4244, AM0627, and Pic using the methods previously outlined (see FIG. 2A). Brackets indicate glycans with only a few examples of cleavage, parentheses indicate that the linkage for the second sialic acid of the disialylated structure could not be assigned. Notably, each mucinase has a unique glycoepitope cleavage motif that is distinct from that of StcE. For instance, BT4244 and Pic cleave N-terminally to a glycosylated Ser or Thr residue, especially those bearing T- or Tn-antigens (represented by the cleavage motif X-S/T*). AM0627 cleaves in between two glycosylated residues bearing similar glycans (represented by the cleavage motif S/T*-S/T*). ZmpC cleaves 4 residues away from a glycosylated Ser or Thr, especially when the glycan contains sialic acid (represented by the cleavage motif S/T*-X-X-X-X (SEQ ID NO:25), where X is any amino acid). Importantly, these cleavage motifs, along with those associated with StcE (S/T*-X-S/T and S/T*-S/T) are minimal cleavage motifs, meaning that further glycosylation beyond the minimal motif can in some cases still result in cleavage. For example, StcE can also cleave S/T*-X-S/T* (Malaker, Pedram, et al. PNAS, 2019, FIG. 2H).

FIG. 35 provides an illustration of an enrichment procedure for enriching mucin-domain glycoprotein. Inactivated and/or point-mutant mucinases are conjugated to beads overnight at 4° C. Sample (lysate, ascites fluid) is added to the beads and bound overnight at 4° C. Beads are washed three times, and then mucin-domain glycoproteins are eluted by boiling in protein loading buffer. The samples are analyzed by western blot or mass spectrometry.

FIGS. 36A-36D. Volcano plots of StcE-enrichment with (FIG. 36A) HeLa lysate, (FIG. 36B) OVCAR3 lysate, and (FIG. 36C) crude cancer-patient ascites fluid (OC235), and of (FIG. 36D) BT4244-enrichment with HeLa lysate. Fold change is shown on the x-axis, and 2.32 indicates >5-fold enrichment of mucins compared to lysate alone. Significance is displayed on the y-axis, where 1.301 designates a p-value of <0.05. Significantly enriched proteins are in the upper-right quadrant, and proteins with a mucin domain are highlighted by enlarged red circles.

FIG. 37. StcE E447D can be used to stain tissues for immunohistochemistry.

FIG. 38. StcE pretreatment of tissues decreases StcE E447D immunohistochemistry staining.

### DETAILED DESCRIPTION

The present disclosure includes the discovery that Secreted Protease of C1 Esterase Inhibitor (StcE), a bacterial protease, cleaves glycoproteins at the peptide backbone by recognizing discrete peptide, glycan-, and secondary structure-based motifs resulting in glycosylated peptide fragments that may be readily analyzed. Among other features, cleavage of glycosylated proteins by StcE provides a powerful tool for the selective proteolysis and analysis of mucin-domain glycoproteins.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

In some aspects, cleavage of mucin-domain glycoproteins by StcE is useful for the structural and functional investigation of mucin-domain glycoproteins. In some aspects, cleavage of mucin-domain glycoproteins by StcE is useful for the breakdown and analysis of secreted and membrane-associated mucin-domain glycoproteins that have been identified as diagnostic and prognostic markers, e.g., in human cancers and in other diseases that are associated with altered or aberrant glycosylations. In some aspects, methods include treating a subject in accordance with a performed analysis as described herein.

Mucin-domain-specific proteolysis, as described herein, facilitates the discovery of unique alterations in disease-associated glycan structures, e.g., the discovery of disease associated “glycosignatures”. Disease associated glycosignatures, as for example cancer glycosignatures, can be utilized in the design and development of diagnostic, prognostic and therapeutic tools. Mucin-domain specific proteolysis, as described herein, facilitates discovery of unique glycosignatures where mucin-domain glycoproteins, e.g., in the host or in an infectious agent, make the host more susceptible to a disease, cause the development of a disease and/or contribute to disease progression. Likewise, the existence of unique glycosignatures may also make a host less susceptible to a disease, prevent the development of a disease and/or prevent disease progression. As such, unique glycosignatures obtained through mucin-domain specific proteolysis facilitates the discovery and development of novel diagnostic and therapeutic tools.

Glycosylation is the enzymatic post-translational addition of carbohydrates (glycans) to proteins and lipids, resulting in “glycoproteins” and “glycolipids,” respectively. Canonically, glycoprotein glycans can be N-linked (linkage to the amide group of Asn) or O-linked (linkage to the hydroxyl group of Ser, Thr). The particular glycan structures, the “glycoforms,” of a glycoprotein impact the function, stability, folding, localization and ligand specificity of the glycoprotein, and play a role in cell adhesion and cell trafficking by modulating how cells interact with each other and with their extracellular matrix environment. The regular process of glycosylation is disrupted during malignant transformation of cells leading to the abnormal, aberrant expression of glycans, that can manifest by, e.g., altered branching and/or truncation of the glycan structures. Aberrantly expressed glycan structures play a crucial role in the pathogenesis and metastasis of solid cancers and hematological cancers. The methods and tools described herein break down areas of dense mucin-type O-glycosylation increasing their availability to interrogation. Proteolytically cleaved fragments of mucin-domain glycoproteins are amenable to analysis using techniques such as gel electrophoresis, column chromatography, mass spectrometry, glycan array, etc.

Mucin-domain glycoproteins (or “mucins”) are a class of heavily glycosylated, high-molecular-mass proteins. They are characterized by the presence of one or more mucin domains, which are enriched in proline, threonine, and serine (PTS) amino acids. The serine and threonine amino acids in these mucin domains (also called “PTS domains”) are heavily modified by glycans pointing out in all directions as bristles, giving them a “bottle-brush” like conformation. Due to the hydroxyl groups of the densely packed saccharide polymers, many mucins have a high capacity to bind water giving them a gel-like consistency. Mucins consist mainly of 0-glycans in which large glycan chains are attached via N-acetylgalactosamine (GalNAc), and often have a high sialic acid content which renders mucins negatively charged in water and increases their rigidity. The complexity and size of the various glycan chains and the thereby resulting variety of mucins provides a high degree of resistance against proteases.

Mucins are present in high density on all mucosal surfaces including the gastrointestinal, respiratory, reproductive, hepatic, pancreatic and renal epithelium, where they function as protection and barriers against extraneous agents, various microbial pathogens and cells.

The generic structure of transmembrane, i.e. membrane-bound, mucins encompasses a mucin domain, glycan side chains, a central protein core (also called mucin protein backbone), a transmembrane domain and a cytoplasmic tail. Secreted mucins contain only a mucin domain, glycan side chains, and a mucin protein backbone.

The human mucin family (MUC) encompasses 21 mucins (MUC 1-21). MUC2, MUC5AC, MUC5B, MUC6, MUC7, MUC8, MUC9 and MUC19 are secreted mucins that protect the epithelium from inflammation, pH changes, toxins and pathogens, while MUC1, MUC3A/B, MUC4, MUC11, MUC12, MUC13, MUC15, MUC16, MUC17, MUC20, MUC21 and MUC22 are transmembrane mucins that may also function as barriers against toxins and pathogens. Aside from the MUC family, there are other 0-glycosylated proteins that are “mucin-type” 0-glycoproteins and characterized by a mucin-domain. As used herein, as described in more detail below, the term “mucin-domain glycoproteins” will generally refer to those proteins recognized as mucins (e.g., belonging to a mucin family) as well as those proteins containing a mucin domain or otherwise recognized as “mucin-type” or “mucin-like”.

Mucin-domain or mucin-type 0-glycoproteins are also present either as secreted or as transmembrane mucins on the surface of nearly every cell in the human body, particularly at outer surfaces that lack an impermeable layer, such as the surfaces of the digestive, genital, and respiratory system tracts. All mucin-domain glycoproteins contain Ser/Thr-linked α-GalNAc as the initiating, anchoring O-linked glycan (0-glycan). The 0-glycan can terminate with a single GalNAc, like the transferrin receptor, or be elaborated to a few dozen O-glycans, like the LDL-receptor, or many dozens, like PSGL-1.

O-linked glycans influence the secondary, tertiary and quaternary structure of protein, and maintain protein stability, heat resistance, hydrophilicity, and protease resistance. Furthermore, O-linked glycans are involved in immunologic recognition, nonspecific protein interactions, receptor-mediated signaling, modulation of the activity of enzymes and signaling molecules, protein expression, and protein processing.

### Role of Mucin-Domain Glycoproteins in Disease Causation, Progression, Diagnosis, and Prognosis

Genetic aberrations, including those due to altered expression of glycan-synthesizing or glycan-modifying enzymes, infections, inflammation and other environmental changes can cause changes in glycosylation. Mucin-domain glycoproteins, as part of the host or as part of an infectious agent, may play a role in causing a disease, progressing a disease, or spreading a disease. Given the vast complexity and variety of glycan structures, particularly of O-linked glycans, in the human body, the identification of glycosignatures may greatly aid in diagnosing those diseases and in prognosing disease progression.

Diseases where altered glycosylation is implicated include, but are not limited to, cancer, viral infections, bacterial infections, autoimmune diseases, and inflammatory diseases.

### Cancers

Both secreted and membrane-bound mucin-domain glycoproteins are detectable by monoclonal antibodies and have gained relevance as diagnostic and prognostic biomarkers in human cancers, e.g., CA-125 and CA19-9, and as potential therapeutic targets and/or vaccines. This is because alterations in mucin-domain glycoprotein expression levels, glycosylation patterns, and sequence aberrations have been found to play a role in cancer growth, cancer progression, metastasis and resistance to cancer treatment, particularly in cancers of epithelial origin, including, but not limited to, breast, ovarian, colorectal, prostate cancer. Tn antigen (GalNAcα1-O-Ser/Thr), Sialyl Tn also known as STn antigen (NeuAcα2-6GalNAcα1-O-Ser/Thr), T antigen (Galβ1-3 GalNAcα1-O-Ser/Thr), and ST (NeuAcα2-Galβ1-3GalNAcα1-O-Ser/Thr) are exemplary O-glycans that are associated with mucin-domain glycoproteins and that have particular relevance as biomarkers, because they occur in a majority of human cancers of various origins, but are generally not expressed in non-cancerous tissues or cells.

### Infections

Mucus is a biopolymer-based hydrogel that lines all moist epithelia of humans and animals. As a physical, tight barrier that prevents microbial pathogens from reaching the underlying epithelial cells, mucus plays a crucial role in the innate immune system or “mucosal immune response”. Mucosal epithelial cells regulate the mucosal immune response by secreting antimicrobial substances and inflammatory mediators, and by modulating antigen-presenting cells and adaptive immune responses, thereby creating organ-specific microenvironments.

Since infections are often caused by airborne pathogens, the microenvironment that is created by the epithelial cells of the respiratory tract is of interest. Access to the respiratory tract is shielded by an airway surface liquid layer that covers the airway surface at the interface between surface epithelial cells and the air space. This airway surface liquid layer comprises a superficial mucus layer that contacts the air space and that covers a periciliary layer which, in turn, contacts surface cilia and the epithelial cells lining the airway. Diffusion of molecules into the periciliary layer is impeded by the membrane-spanning mucins and mucopolysaccharides within the superficial mucus layer.

### Viral Infections

Infections with representative infectious agents from virus families and species including, but not limited to, the Ebola virus species, retroviruses including, but not limited to, the HIV-1 or HIV-2 families, Herpes virus families may be detected by identifying glycosignatures that reveal alterations in mucin-domain glycoprotein expression levels, glycosylation patterns, and/or sequence aberrations in the infectious agent.

Mucins such as MUC1, MUC4, MUC5B, MUC7, and mucin-domain glycoproteins occurring in human breastmilk, saliva and cervical plug have been described to inhibit HIV infection in-vitro, and may also function as barriers against infection with HIV-1 and HIV-2 in vivo, and against infections with poxvirus. Proteolytic analysis of the glycans involved as to identify patterns and sequences that provide such protection facilitates the development of diagnostic and therapeutic tools to prevent the infection with HIV-1 and/or HIV-2, and other retroviruses.

Furthermore, isolated gastric mucin polymers (e.g., isolated porcine gastric mucin polymers), which are key structural components of native mucus, reportedly protect underlying cell layers from infection by viruses (e.g., small viruses) such as human papillomavirus Merkel cell polyomavirus (MOT), or a strain of influenza A virus, and may also inhibit rotaviruses and noroviruses.

The Ebola virus (EBOV) species including, but not limited to, Zaire (ZEBOV), Sudan, Côte d'Ivoire, Reston (REBOV) and ‘Bundibugyo’ encompass filoviruses that cause severe, potentially life-threatening, hemorrhagic fever in humans and non-human primates, and for which currently no approved treatment is available. The Ebola virus glycoprotein, which exhibits a prominent mucin domain, is presumed to be responsible for binding and fusion of the virus with host cells. Upon infection of the host, very few neutralizing antibodies are elicited which is believed to be due to excessive glycosylation.

Heparan sulfate proteoglycans are present on the surface of most types of vertebrate cells. Both herpes simplex virus types 1 and 2 (HSV-1 and HSV-2) initiate infection by binding to cell surface heparan sulfate, but mucus can interfere with the binding, for example, by trapping the viruses.

Members of the Paramyxoviridiae family (respiratory RNA viruses) and of the Orthomyxoviridiae family (influenza viruses) have also been reported to manipulate mucus secretion and cause major obstruction of the airways.

### Bacterial Infections

Infections with representative infectious agents from bacterial families that are known to cause pathologies may be detectable, treatable or preventable by identifying glycosignatures that reveal alterations in mucin-domain glycoprotein expression levels, glycosylation patterns, and/or sequence aberrations in the infectious agent.

Cystic fibrosis (CF) is a genetic, ultimately fatal disease where a mutation in the CF transmembrane conductance regulator gene causes a hypersecretion of mucus in organs, particularly in the lungs, where the excessive amount of mucus clogs the airways and traps microbial pathogens leading to lung damage and ultimately respiratory failure. Among the microbial pathogens, Pseudomonas aeruginosa is the primary bacterial cause of chronic pneumonia in cystic fibrosis patients and is also thought to further increase mucus secretion. Information gathered from glycosignatures may be used counteract such hypersecretion of mucus.

Tuberculosis is a severe and possibly life-threatening respiratory disease caused by transfection with Mycobacterium tuberculosis, an airborne pathogen, via the respiratory mucosa. Although reportedly every third person carries Mycobacterium tuberculosis, only about 10% of those carriers develop tuberculosis, which means that the majority of infections with Mycobacterium tuberculosis remain dormant, most likely due to the mucosal immune response.

Infective endocarditis is a life-threatening cardiovascular disease in which blood-borne microbial pathogens attach to and colonize in platelet-fibrin thrombi on cardiac valve surfaces. Microbial pathogens expressing cell surface serine-rich repeat glycoproteins (adhesins) containing “Siglec-like” binding regions, for example Siglec-like streptococcal adhesins, have been reported to play a role in the pathogenesis of infective endocarditis, because the “Siglec-like” binding regions on the pathogens were found to have a significant impact on the degree of colonization and virulence, depending on their interaction with mucin-type O-glycosylated subsets of plasma glycoproteins (sialylated proteins) such as proteoglycan 4 (PRG4), inter-alpha-trypsin inhibitor heavy chain H4 (ITIH4), C1 esterase inhibitor (C1-INH), and GPIbα. Tools for breaking down and analyzing O-linked glycoproteins, as described herein, will facilitate the analysis and identification of sialylated proteins in plasma which contribute to the pathogenesis of infective endocarditis. Note, as well, that since many of those sialylated proteins described above are biomarkers for disorders and diseases including, but not limited to: rheumatoid arthritis, chronic obstructive pulmonary disease, obesity, type-2 diabetes, stroke, depression, hepatic fibrosis, thrombocytopenia, pre-eclampsia, hereditary angioedema, cancers, the tools for breaking down and analyzing O-linked glycoproteins, as described herein, facilitate the analysis and identification of sialylated proteins in those disorders and diseases as well and improve the diagnosis, prognosis and monitoring of therapeutic success.

Chronic infection with Helicobacter pylori and subsequent gastric tissue inflammation have been reported to cause a change in the gastrointestinal glycan repertoire. Likewise, in cases of long-lasting inflammation such as in inflammatory bowel disease (ulcerative colitis, Chrono's disease) alterations in glycosylation were reported which were sometimes reversible, once inflammation had ceased.

### Auto-Immune Diseases

Cutaneous lupus erythematosus (LE) is an incompletely understood autoimmune disease that is characterized by increased dermal mucin containing various glycoproteins as well as glycosaminoglycans such as hyaluronic acid and chondroitin sulfate. The occurrence of dermal mucin is often used to differentiate LE from other inflammatory dermatitides. A more in-depth and specific analysis of the dermal mucin using the tools described herein facilitates the diagnosis and treatment of LE.

Rheumatoid arthritis (RA) is a systemic autoimmune disease characterized by infiltration of lymphocytes and macrophages into the synovium and abnormal synovial hyperplasia, synovial fluid and synovial tissues from patients with inflamed knee, elbow, and hip joints due to rheumatoid arthritis were found to contain various mucin-domain glycoproteins including MUC1 which may suggest that MUC1 and similar glycoproteins may be new targets for treatment of rheumatoid arthritis.

Multiple sclerosis (MS) is an inflammatory, demyelinating disease of the central nervous system that is characterized by chronic neuroinflammation and progressive neurodegeneration. Recently, family members of T cell Ig- and mucin-domain molecules (TIMs), expressed on T cells and that are involved in the regulation of the innate immune response, have been observed to be differentially expressed in multiple sclerosis, and are thought to be involved in the etiology of autoimmune and allergy diseases.

### Ocular Surface Diseases

Ocular surface mucins play a critical role in the protection of corneal and conjunctival epithelia and the tools described herein facilitate the identification of glycoprotein changes in ocular surface diseases.

As described herein, StcE possesses unique properties to map glycosylation sites and structures on purified and recombinant human mucin-domain glycoproteins, including cancer-associated mucin-domain glycoproteins from cultured cells and from ovarian cancer patient-derived ascites fluid, by mass spectrometry. The present disclosure also describes methods for investigating mucin-binding receptors and their biological ligands, which is exemplified herein by the discovery that Siglec-7, a glyco-immune checkpoint receptor, specifically binds sialomucins as biological ligands, whereas the related Siglec-9 receptor does not.

Before describing these specific embodiments of the disclosure, it will be helpful to set forth definitions that are used in describing the present disclosure.

### Definitions

Unless defined otherwise, all technical and scientific terms used herein have the same meaning as commonly understood by a person of ordinary skill in the art to which this invention belongs. The following definitions are intended to also include their various grammatical forms, where applicable. As used herein, the singular forms “a,” “an,” or “the” include plural referents, unless the context clearly dictates otherwise. Thus, for example, reference to “a cell” includes a plurality of such cells and reference to “the agent” includes reference to one or more agents known to those skilled in the art, and so forth.

The term “about” in relation to a reference numerical value can include a range of values plus or minus 10% from that value. For example, the amount “about 10” includes values from 9 to 11, including the values of 9, 10, and 11. The term “about” in relation to a reference numerical value can also include a range of values plus or minus 10%, 9%, 8%, 7%, 6%, 5%, 4%, 3%, 2%, or 1% from that value.

The terms “mucinase” and “mucin-specific protease,” as used herein, refers to molecules that proteolytically cleave mucin-domain glycoproteins.

The term “nucleic acid,” “nucleotide,” or “polynucleotide,” as used herein, refers to deoxyribonucleic acids (DNA), ribonucleic acids (RNA) and polymers thereof in either single-, double- or multi-stranded form. The term includes, but is not limited to, single-, double- or multi-stranded DNA or RNA, genomic DNA, cDNA, DNA-RNA hybrids, or a polymer comprising purine and/or pyrimidine bases or other natural, chemically modified, biochemically modified, non-natural, synthetic or derivatized nucleotide bases. In some embodiments, a nucleic acid can comprise a mixture of DNA, RNA and analogs thereof. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues.

The terms “polypeptide,” “peptide,” and “protein” are interchangeably used in some instances herein and refer to a polymer of amino acid residues, or an assembly of multiple polymers of amino acid residues. In some instances, a peptide may refer to one or more cleaved portions generated from a longer polypeptide. The term “peptide” will include single linear chains of many amino acids of varied length, including but not limited to e.g., dipeptides, tripeptides, tetrapeptides, pentapeptides, hexapeptides, heptapeptides, octapeptides, nonapeptides, decapeptides, etc., as well as peptides from about 2 to over about 100 residues in length, including but not limited to e.g., from 2 to 100 residues, from 2 to 75 residues, from 2 to 50 residues, from 2 to 25 residues, from 2 to 20 residues, from 2 to 15 residues, from 2 to 10 residues, from 5 to 100 residues, from 5 to 75 residues, from 5 to 50 residues, from 5 to 25 residues, from 5 to 20 residues, from 5 to 15 residues, from 10 to 100 residues, from 10 to 75 residues, from 10 to 50 residues, from 10 to 25 residues, from 10 to 20 residues, from 10 to 15 residues, and the like.

The term “amino acid” includes, but is not limited to, naturally-occurring α-amino acids and their stereoisomers. “Stereoisomers” of amino acids refer to mirror image isomers of the amino acids, such as L-amino acids or D-amino acids. For example, a stereoisomer of a naturally-occurring amino acid refers to the mirror image isomer of the naturally-occurring amino acid (i.e., the D-amino acid).

Naturally-occurring α-amino acids are those encoded by the genetic code as well as those amino acids that are later modified (e.g., hydroxyproline, γ-carboxyglutamate, and 0-phosphoserine). Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

The 20 amino acids that are encoded by the triplet codons of the genetic code include (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr).

Amino acids may be referred to herein by either their commonly known three letter symbols or by the one-letter symbols recommended by the IUPAC-IUB Commission on Biochemical Nomenclature. For example, an L-amino acid may be represented herein by its commonly known three letter symbol (e.g., Arg for L-arginine) or by an upper-case one-letter amino acid symbol (e.g., R for L-arginine). A D-amino acid may be represented herein by its commonly known three letter symbol (e.g., D-Arg for D-arginine) or by a lower-case one-letter amino acid symbol (e.g., r for D-arginine).

The terms “deglycosylating” and “to deglycosylate,” as used herein, generally refer to removing glycans (N-glycans, O-glycans) from a protein.

The terms “deglycosylated protein” or “deglycosylated polypeptide,” as used herein, refer to a polypeptide that was at one point glycosylated, but has been exposed to a deglycosylating enzyme or chemical mixture under deglycosylating conditions to reduce the number of glycans or to entirely eliminate attached glycans.

The term “glycan,” as used herein, refers to monomers as well as polymers of saccharide residues, including, but not limited to, naturally occurring residues, e.g., glucose, N-acetylglucosamine, N-acetyl neuraminic acid, galactose, mannose, fucose, hexose, arabinose, ribose, xylose, and modified residues, e.g., 2′-fluororibose, 2-deoxy-ribose, phosphomannose, 6′-sulfo-N-acetylglucosamine Glycans play key roles in a variety of biological processes ranging from vascularization, immunity, differentiation to cellular communication, and the glycosylation pathways are closely regulated through the activity of glycosyltransferases and glycosidases which synthesize and modify glycans.

Glycan structures can be linear or branched, and can be composed of homopolymers or heteropolymers of oligosaccharide residues. Glycans can occur as free glycans (e.g., hyaluronan), as components of glycoconjugates or as glycans that were released from glycoconjugates. Glycoconjugates are molecules in which at least one saccharide moiety is covalently linked to at least one other moiety, such as a lipid or a protein, and include, but are not limited to, N-linked glycoproteins, O-linked glycoproteins, glycolipids, proteoglycans, etc.

The saccharide moieties may be in the form of monosaccharides, disaccharides, oligosaccharides, and/or polysaccharides, may comprise branched or unbranched chains of saccharide residues, and may include sulfyl- or phosphor-modifications as well as acetyl-, glycolyl-, propyl- or other alkyl modifications. The term “glycan” also encompasses sialic acids, which are a class of glycans with a shared nine-carbon backbone and which are often attached to the terminal positions of several classes of cell-surface and secreted N- and O-linked glycans. Sialylated ligands are recognized by sialic acid-binding proteins such as the family of Selectins (vascular adhesion molecules), Siglecs, L1 cell-adhesion molecule (L1CAM) and factor H. The term “glycan” also includes glycosaminoglycans (GAGs), such as heparin, heparan sulfate, hyaluronic acid, chondroitin sulfate, dermatan sulfate, and keratan sulfate, which are long carbohydrate chains consisting of repeating disaccharide units. In proteoglycans, glycosaminoglycans are covalently attached to a protein core. Sialic acid residues and glycosaminoglycan are frequently targeted by different viruses to assist their attachment to susceptible host cells. Furthermore, glycosaminoglycans are reported to function immunologically by activating macrophages, dendritic cells, and neutrophils, and may also have an effect on tumor necrosis factor alpha and interleukin-6.

Standard symbol nomenclature for glycans has been established and is widely employed and available in the relevant art. See e.g., Symbol Nomenclature for Glycans (SNFG) described in Glycobiology (2015) 25: 1323-1324, and available online at www(dot)ncbi(dot)nlm(dot)nih(dot)gov/glycans/snfg(dot)html.

The term “N-glycan” refers to a glycan linked to the glycoconjugate via a nitrogen linkage (N-linked glycan). Generally, N-glycans are linked to the amino side chain of an asparagine residue.

The term “O-glycan” refers to a glycan linked to the glycoconjugate via an oxygen linkage (O-linked glycan). Mucin-type O-glycans are defined as O-glycans that are linked to the side chain of a serine or threonine residue via the addition of an N-acetylgalactosamine (GalNAc) residue. The glycan chain, once containing GalNAc, can be further extended by adding other monosaccharides (GalNAc, N-acetylglucosamine or GlcNAc, galactose, mannose, fucose, xylose etc).

The term “glycoprotein,” as used herein, refers to a polypeptide sequence that is associated with one or more carbohydrate (sugar, saccharide, oligosaccharide, or as most commonly used, glycan) structures. Glycoproteins can have distinct “glycosignatures” depending on the particular glycan structures that a polypeptide sequence is associated with. Such glycosignatures refer to the set of glycan structures that is present in a population of glycoproteins or fragments thereof along with the associated context, e.g. attachment sites, of the glycan structures. Glycosignatures can, e.g., in comparison to a reference or control profile or signature, serve as biomarkers to indicate the presence of a condition, such as a disease, where glycoproteins play a role in causing the condition, e.g., in disease progression, disease spread, and the like. Glycosignatures may be useful in “glycoproteomics”, which involves the analysis of the glycosylation in the context of the protein backbone to which glycans are attached, e.g., analysis of a glycoprotein, including the backbone amino acid sequence and the position and identity of attached glycans. In comparison, “glycomics”, as commonly referred to in the art, involves removal and identification of glycans, including e.g., all glycans, of a sample outside the context of the protein backbone to which such glycans were attached (i.e., glycan attachment information, including the position of attachment, is generally not retained).

Glycosignatures can be characterized by various parameters including, but not limited to, the composition and identity of analyzed glycans, the specific amino acid or sequence sites occupied, the presence of glycans of a particular type either in isolation or in combination, the degree of occupancy of glycosylation sites, the three-dimensional arrangement of the glycans relative to one another and the protein backbone, and combinations of such parameters. In some instances, glycosignatures may include quantitation of glycoproteins or the glycosylation thereof, including e.g., where such quantitation is relative, e.g., increased or decreased, relative to a reference or control. In some instances, glycosignatures may include qualitative measures. Herein, the term “glycosignatures” may be interchangeably used with any of the terms “glycoprofiles,” “glycosylation profiles,” or “glycosylation patterns.”

The term “sugar” or “carbohydrate,” as used herein, encompasses any of a class of aldehyde or ketone derivatives of polyhydric alcohols, including, but not limited to, mannitol, sorbitol, xylitol, maltitol, lactitol, erythritol, arabitol, ribitol, glucose, fructose, mannose, galactose, lactose, sucrose, raffinose, ribitol, maltose, sorbose, cellobiose, sorbose, trehalose, maltodextrins, dextrans, inulin, 1-O-alpha-D-glucopyranosyl-D-mannitol.

The terms “mucin-domain glycoprotein” and “mucin-type glycoprotein” are interchangeably used herein, and encompass any glycoprotein that is characterized by a mucin domain and, as such, contains Ser/Thr-linked α-GalNAc as the initiating, anchoring O-linked glycan (0-glycan). The 0-glycan can terminate with a single GalNAc or be elaborated to a few dozen O-glycans.

Podocalyxin, MUC16, PSGL-1, Syncam-1, CD43, and CD45 are non-limiting examples of mucin-domain glycoproteins. Podocalyxin is a major sialoprotein in the glycocalyx of the podocytes in the kidney glomerulus, and reportedly promotes the growth and proliferation of solid tumors and enhances the metastasis of solid tumors. MUC16 is expressed by normal bronchial, endometrial, ovarian and corneal epithelial cells. MUC16 has been found to function as a barrier against bacterial and viral infections in ocular epithelia. In addition, MUC16 can be overexpressed in cancerous cells (especially in ovarian cancer). This overexpression facilitates evasion of these cells from detection and/or eradication by innate immune cells. The engagement of P-selectin glycoprotein ligand-1 (PSGL-1), a sialomucin, leads to the activation of several signaling pathways that are involved in the innate immune response, involving monocytes, macrophages, microglial cells, MAPK, NF-KB, and more. SynCams such as SynCam-1 are synaptic cell adhesion molecules that trigger synaptogenesis and contribute to synaptic organization and maintenance. SynCams belong to the immunoglobulin superfamily (IgSF) which are linked to the plasma membrane via a single transmembrane domain or via a glycosyl-phosphatidyl anchor, whereby glycosylation regulates their cis- and trans-interactions.

Endogenous glycan-binding proteins such as the c-type lectins, sialic acid-binding immunoglobulin-like lectins (siglecs), and galectins (Gal), are important in facilitating vascular signaling, immune cell activation or suppression, and immune cell viability. For example, Gal-1 can engage apoptotic programs through binding to N- and O-glycans present in CD45, CD43, and CD7.

The terms “isolating,” “separating,” and “purifying,” as used herein, refer to the separation of a polynucleotide, protein, glycan, cell, or other component in a sample, thereby substantially enriching the component. For example, in the context of a deglycosylation reaction, isolating the free glycans, particularly O-glycans, means separating the free glycans from the deglycosylated protein and the deglycosylating enzyme by various adsorption or affinity methodologies, known in the art, that are based on size, charge etc. of the various, to-be-separated components.

The term “glycosite,” as used herein, refers to the specific amino acid that bears glycosylation.

The term “glycomapping,” as used herein, is defined as the complete analysis of a protein and its associated glycans. Specifically, this involves amino acid sequence determination, tabulation of all glycans present on the protein, and site localization of all glycans to all glycosites. Glycomapping may be used in some instances to identify particular glycosignatures.

The term “sample,” as used herein, refers to any solid or fluid sample obtained from any living cell or organism, including, but not limited to, human or animal tissue, organ, tissue culture, bioreactor sample, eukaryotic organism, prokaryotic organism. For example, a sample can be obtained from, e.g., blood, plasma, serum, urine, sputum (saliva), bile, seminal fluid, cerebrospinal fluid, vitreous humor, aqueous humor, any bodily secretion, transudate, exudate. Essentially any convenient and appropriate sample may find use in the subject methods and, correspondingly, any convenient sampling or sample collection method may be utilized.

As used herein, the term “recombinant” refers to a polypeptide derived from genetic material that has been modified using methods well known in the art. The term “recombinant” can also be applied to cells, tissues, and organisms in which genetic modifications have been made. The modified genetic material (polynucleotides) may also be referred to as “recombinant.”

The terms “subject,” “individual,” and “patient” are used interchangeably herein to refer to a vertebrate, e.g., a mammal, e.g., a human. Mammals include, but are not limited to, rodents (e.g., mice, rats), simians, humans, farm animals, and pets. Tissues, cells, and their progeny of a biological entity obtained in vivo or cultured in vitro are also encompassed.

The term “codon optimization” refers to altering a nucleic acid sequence, without changing the encoded amino acid sequence, in such a way that codon bias (i.e., the preferential use of particular codons that can vary between species) is reduced or rebalanced. In some embodiments, codon optimization increases translational efficiency. As a non-limiting example, leucine is encoded by six different codons, some of which are rarely used. By rebalancing codon usage (e.g., within a reading frame), preferred leucine codons can be selected over rarely used codons. The nucleic acid sequence encoding the protein of interest is altered such that the rarely used codons are converted to preferred codons. Rare codons can be defined, for example, by using a codon usage table derived from the sequenced genome of a host species (i.e., the species in which the protein will be expressed). Codon optimization may also be employed to modulate GC content, e.g., to increase mRNA stability or reduce secondary structure; or otherwise minimize codons that may result in stretches of sequence that impair expression of the protein of interest.

The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=# of identical positions/total # of positions×100). When a position in one sequence is occupied by the same nucleotide as the corresponding position in the other sequence, then the molecules are identical at that position.

In some instances, a polynucleotide or peptide of the present disclosure may have at least about 70% identity (e.g., sequence identity), including but not limited to e.g., at least about 85%, 86%, 87%, 88%, 89%, 90%, 91%, 92%, 93, 94%, 95%, 96%, 97%, 98%, 99%, or 100% identity, to a reference sequence, such as a sequence provided herein. Such comparisons may be made where the sequences are aligned for maximum correspondence over a comparison window, or designated region as measured using a sequence comparison algorithm or by manual alignment and visual inspection

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence similarities for the test sequences relative to the reference sequence, based on the program parameters. For sequence comparison of nucleic acids and proteins, the BLAST and BLAST 2.0 algorithms can be used.

Methods of alignment of sequences for comparison are well-known in the art. Optimal alignment of sequences for comparison can be conducted, e.g., by the local homology algorithm of Smith & Waterman (Adv. Appl. Math., 2, 482-489 (1981)), by the homology alignment algorithm of Needleman & Wunsch (J Mol Biol 48, 443-453 (1970)), by the search for similarity method of Pearson & Lipman (Proc Natl Acad Sci USA 85, 2444-2448 (1988)), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (1995)).

Additional examples of algorithms that are suitable for determining percent sequence similarity or identity are the BLAST and BLAST 2.0 algorithms. Software for performing BLAST analyses is publicly available at the National Center for Biotechnology Information website, ncbi(dot)nlm(dot)nih(dot)gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix.

The BLAST algorithm also performs a statistical analysis of the similarity and/or identity between two sequences. One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The terms “disease,” “disease condition,” and “condition,” as used herein, are used interchangeably herein, and refer to any disease where mucin-domain glycoproteins, for example, as part of the host or as part of an infectious agent, play any role in causing the disease, progressing the disease, or spreading the disease. Diseases that are contemplated in this context include cancer; viral infections including, but not limited to, infections with representative pathogens from the Ebola virus families, HIV-1 or HIV-2 families, Herpes virus families; bacterial infections including, but not limited to, tuberculosis, inflammatory bowel disease, infective endocarditis; and autoimmune diseases including, but not limited to, rheumatoid arthritis, multiple sclerosis, lupus erythematosus.

The term “cancer,” as used herein, refers to any of various malignant neoplasms characterized by the proliferation of anaplastic cells that tend to invade surrounding tissue and metastasize to new body sites. Non-limiting examples of different types of cancer suitable for identification and study according to methods and compositions of the present disclosure include skin cancer (e.g., melanoma), colorectal cancer, colon cancer, anal cancer, liver cancer, ovarian cancer, breast cancer, lung cancer, bladder cancer, thyroid cancer, pleural cancer, pancreatic cancer, cervical cancer, prostate cancer, testicular cancer, bile duct cancer, gastrointestinal carcinoid tumors, esophageal cancer, gall bladder cancer, rectal cancer, appendix cancer, small intestine cancer, stomach (gastric) cancer, renal cancer (i.e., renal cell carcinoma), cancer of the central nervous system, oral squamous cell carcinoma, choriocarcinomas, head and neck cancers, bone cancer, osteogenic sarcomas, fibrosarcoma, neuroblastoma, glioma, melanoma, leukemia (e.g., acute lymphocytic leukemia, chronic lymphocytic leukemia, acute myelogenous leukemia, chronic myelogenous leukemia, or hairy cell leukemia), lymphoma (e.g., non-Hodgkin's lymphoma, Hodgkin's lymphoma, B-cell lymphoma, or Burkitt's lymphoma), and multiple myeloma. The cancer can be any stage (e.g., advanced cancer or metastatic cancer).

### Methods

As summarized above, the present disclosure includes methods involving the cleavage of a mucin-domain glycoprotein by a mucin-specific protease, including e.g., where the mucin specific protease is a secreted protease of C1 esterase inhibitor (StcE) or an analog, mutant, or derivative thereof. Useful StcE proteins include but are not limited to e.g., a StcE having at least 90% sequence identity with SEQ ID NO:1, e.g., a StcE having 100% sequence identity with SEQ ID NO:1, a recombinant StcE variant having less than 100% sequence identity with SEQ ID NO:1, and the like.

The present disclosure also includes methods involving the cleavage of a mucin-domain glycoprotein by a mucin-specific protease, including e.g., where the mucin specific protease is a serine peptidase mucinase, a zinc metallopeptidase mucinase, an analog, mutant, or derivative thereof. Useful serine peptidase mucinase include but are not limited to e.g., a Pic polypeptide having at least 90% sequence identity with SEQ ID NO:17, e.g., a Pic polypeptide having 100% sequence identity with SEQ ID NO:17. Useful zinc metallopeptidase mucinase include but are not limited to mucinases of Family M26, Family M60, or Family M66. For example, methods involving the cleavage of a mucin-domain glycoprotein by a mucin-specific protease may include using a ZmpC polypeptide having at least 90% sequence identity with SEQ ID NO:18, e.g., a ZmpC polypeptide having 100% sequence identity with SEQ ID NO:18; a BT4244 polypeptide having at least 90% sequence identity with SEQ ID NO:19, e.g., a BT4244 polypeptide having 100% sequence identity with SEQ ID NO:19; a AM0627 polypeptide having at least 90% sequence identity with SEQ ID NO:20, e.g., a AM0627polypeptide having 100% sequence identity with SEQ ID NO:20; a AM0908 polypeptide having at least 90% sequence identity with SEQ ID NO:21, e.g., a AM0908 polypeptide having 100% sequence identity with SEQ ID NO:21; a AM1514 polypeptide having at least 90% sequence identity with SEQ ID NO:22, e.g., a AM1514 polypeptide having 100% sequence identity with SEQ ID NO:22; a SmEnhancin polypeptide having at least 90% sequence identity with SEQ ID NO:23, e.g., a SmEnhancin polypeptide having 100% sequence identity with SEQ ID NO:23; a VIBHAR2194 having at least 90% sequence identity with SEQ ID NO:24, e.g., a VIBHAR2194 polypeptide having 100% sequence identity with SEQ ID NO:24.

Cleavage of mucin-domain glycoprotein by a mucinase will generally involve the cleavage of a mucin-specific glycan-peptide cleavage motif. By “mucin-specific glycan-peptide cleavage motif” is generally meant a sequence having a specific arrangement of amino acid residues (or an amino acid residue motif) that includes specific glycosylation within the motif. As such, both the particular amino acid residues and the glycosylation are recognized by a mucinase, such as but not necessarily limited to StcE, to initiate cleavage of at the mucin-specific glycan-peptide cleavage motif.

In some instances, a mucin-specific glycan-peptide cleavage motif recognized by a mucin-specific protease is S/T*-X-S/T, wherein * denotes glycosylation of the S or T residue and X is any amino acid residue or absent. Accordingly, examples of glycan-peptide sequence motifs that may be recognized include, but may not be limited to, e.g., S*-S, S*-T, S*-X-S, S*-X-T, T*-S, T*-T, T*-X-S, T*-X-T, and the like, where X (where present) may be any amino acid residue.

In some instances, methods of the present disclosure may include detecting a mucin-domain glycoprotein that includes a mucin-specific glycan-peptide cleavage motif. Such detection may be performed, or other methods may involve, contacting a sample with a mucinase, where the specific mucinase employed may vary, to generate glycopeptides by cleaving mucin-domain glycoproteins present in the sample.

Analysis of the sample to detect and identify cleaved proteins in some instances facilitates identification of those proteins as mucin-domain glycoproteins.

Visualization of the sample to detect expression of a mucin-domain glycoprotein may be useful to identify cells and tissues that express mucin-domain glycoproteins. For example, the sample may be cells isolated from a subject or tissue samples from a subject. The mucinase may be labeled, e.g., by conjugation to a label. The label may be an optically detectable label, e.g., a fluorescent or a luminescent label. For example, the mucinase may be conjugated to a fluorophore, such as, Cy-3, Cy-5, Quasar 570, Quasar 670, Alexafluor555, Alexafluor647, BODIPY V-1002, BODIPY V1005, POPO-3, TOTO-3, POPRO3, or TOPRO3.

As summarized above, various samples may be employed in the herein described methods, including proteinaceous samples, cellular samples and the like. Proteinaceous samples may be substantially or entirely acellular and may, in some instances, consist substantially or entirely of protein. In some instances, proteinaceous samples may consist primarily of protein buy may include other members, including e.g., other biomolecules. Cellular samples may be derived from living tissues or collections of cultured cells or the like. Cellular samples may be heterogeneous, containing various (including 2 or more, 3 or more, 4 or more, 5 or more, etc.) different types of cells, or may substantially homogeneous, containing essentially one type of cell, depending on the source from which the cellular sample is derived.

Samples used in the methods of the present disclosure may be collected by any convenient means. In some instances, useful cellular samples may be or may be derived from a biopsy. Biopsy tissues may be obtained from healthy or diseased tissues, including e.g., cancer tissues. Depending on the type of cancer and/or the type of biopsy performed the sample may be prepared from a solid tissue biopsy or a liquid biopsy.

In some instances, a sample may be prepared from a surgical biopsy. Any convenient and appropriate technique for surgical biopsy may be utilized for collection of a sample to be employed in the methods described herein including but not limited to, e.g., excisional biopsy, incisional biopsy, wire localization biopsy, and the like. In some instances, a surgical biopsy may be obtained as a part of a surgical procedure which has a primary purpose other than obtaining the sample, e.g., including but not limited to tumor resection, mastectomy, lymph node surgery, axillary lymph node dissection, sentinel lymph node surgery, and the like.

Various other biopsy techniques may be employed to obtain biopsy tissue, for use as a sample as described herein. As a non-limiting example, a sample may be obtained by a needle biopsy. Any convenient and appropriate technique for needle biopsy may be utilized for collection of a sample including but not limited to, e.g., fine needle aspiration (FNA), core needle biopsy, stereotactic core biopsy, vacuum assisted biopsy, and the like.

Essentially any convenient and appropriate biological sample, cellular or acellular, may be employed in the herein described methods. Accordingly, various different sampling methods and/or sample collection procedures may be employed. In some instances, the instant methods may involve one or more biopsy collection methods described above. In some instances, methods of the present disclosure may involve one or more liquid biological samples and/or include one or more methods for collecting a liquid sample from a subject. Various biological fluids, including but not limited to e.g., amniotic fluid, aqueous humour, vitreous humour, bile, blood, blood plasma, blood serum, cerebrospinal fluid, cerumen, chyle, chyme, endolymph, perilymph, exudates, feces, gastric juice, lymph, mucus, pericardial fluid, peritoneal fluid, pleural fluid, pus, rheum, saliva, sebum, serous fluid, semen, serum, smegma, sputum, synovial fluid, sweat, tears, urine, vaginal secretion, vaginal discharge, vomit, and the like, may be employed and/or any appropriate method of collection corresponding to the subject fluid, e.g., paracentesis to collect peritoneal fluid or ascites fluid, may be utilized.

Methods of the present disclosure may include or exclude one or more steps for separating components obtained from or produced in a sample, including e.g., enriching and/or isolating components of an obtained or produced sample. For example, in some instances, a method described herein may include enriching a sample for intact glycoproteins or cleaved glycopeptides or isolating intact glycoproteins or cleaved glycopeptides from a sample, including where such cleaved glycopeptides are produced through the cleavage of a mucin-specific glycan-peptide cleavage motif present in a mucin-domain glycopeptide. Intact glycoproteins may be bound and/or separated through the use of a catalytically inactive mucin-specific protease, as described herein. Where employed, one or more steps for separating (e.g., enriching and/or isolating and/or depleting) components of an obtained or produced sample may be performed at any convenient and appropriate point in the procedure. For example, in some instances, sample is enriched for intact glycoproteins, generated glycopeptides, or generated glycopeptides are isolated prior to analyzing the generated glycopeptides, including but not limited to e.g., where the generated glycopeptides are analyzed by mass spectrometry. One or more separation steps may also provide for depleting a sample of a particular component, including but not limited to e.g., depleting a sample of albumin, other contaminating non-glycoproteins, one or more mucin-domain glycoproteins, or other intact glycoproteins and/or cleaved glycopeptides.

In some instances, methods of the present disclosure involving separation (e.g., enrichment and/or isolation) may employ a mucinase lacking protease activity. A mucinase lacking protease activity may be referred to as an enzymatically “dead”, a mucinase mutant, catalytically inactive, or a non-functional mucinase.

In some instances, methods of the present disclosure may include contacting a sample with catalytically inactive mucinase, such as but not limited to e.g., a catalytically inactive secreted protease of C1 esterase inhibitor (StcE), including but not limited to e.g., a catalytically inactive StcE having at least 90% sequence identity with SEQ ID NO:1. In some instances, useful catalytically inactive versions of mucinase proteins may include recombinant mucinase proteins modified to be catalytically inactive, e.g., by mutation including, deletion, insertion, or substitution mutation. Useful catalytically inactive versions of StcE include but are not limited to e.g., StcE E447D mutant (e.g., as described in Yu et al., Structure. (2012) 20(4):707-17; the disclosure of which is incorporated herein by reference in its entirety). Other useful catalytically inactive forms of StcE include those generated by targeted or random mutagenesis or rational design, including e.g., those generated with or without use of available crystal structures of StcE proteins such as e.g., PDB: 3UJZ and 4DNY.

Additional examples of catalytically inactive mucinase include mucinases having a deletion or a substitution in a catalytic domain. Useful catalytically inactive mucinases include mucinases having an amino acid sequence at least 80% identical to the amino acid sequence of any one of SEQ ID NOs: 1 and 17-24. In certain aspects, a catalytically inactive mucinase may include the substitution at position E575 (e.g., E575A substitution) with reference to the amino acid sequence of SEQ ID NO:19. In certain aspects, a catalytically inactive mucinase may include the substitution at position E326 (e.g., E326A substitution) with reference to the amino acid sequence of SEQ ID NO:20.

In some instances, a protease inhibitor may be employed in methods involving enrichment or isolation procedures. Accordingly, catalytically inactive mucin-specific proteases will include otherwise active mucinases in the presence of a protease inhibitor. Useful protease inhibitors may include but are not limited to e.g., AEBSF (4-(2-aminoethyl)benzenesulfonyl fluoride hydrochloride), 6-aminohexanoic acid, antipain, aprotinin, benzamidine HCl, bestatin, chymostatin, E-64, EDTA (ethylenediaminetetraacetic acid, including salts thereof, e.g., the disodium salt), N-ethylmaleimide, leupeptin, pepstatin, phosphoramidon, trypsin inhibitor, and the like.

Accordingly, in some instances, methods of the present disclosure may involve contacting a sample with a mucinase and a protease inhibitor or contacting a sample with a composition comprising a mucinase and a protease inhibitor, where the protease inhibitor inhibits the protease activity of the mucinase. For example, in some embodiments, methods may involve contacting a sample with a mucinase and EDTA or contacting a sample with a composition comprising a mucinase and EDTA, where the EDTA inhibits the protease activity of the mucinase. In some instances, methods may involve contacting a sample with a StcE protein and a protease inhibitor or contacting a sample with a composition comprising a StcE protein and a protease inhibitor, where the protease inhibitor inhibits the protease activity of the StcE protein. In some instances, methods may involve contacting a sample with a StcE protein and EDTA or contacting a sample with a composition comprising a StcE protein and EDTA, where the EDTA inhibits the protease activity of the StcE protein.

Methods employing a catalytically inactive mucinase and/or a mucinase in the presence of a reagent or solution sufficient to inhibit the protease activity of a mucinase (such as a protease inhibitor or a solution containing a protease inhibitor) may be employed for various purposes. For example, in such methods a sample may be enriched for glycoproteins that bind to the inactive mucinase or the binding activity of the inactive mucinase may be employed to isolate, detect and/or identify one of more glycoproteins that bind to the inactive mucinase. In some instances, binding of an inactive mucinase to a glycoprotein may be employed to deplete a sample of the glycoprotein. Accordingly, due to the catalytic inactivity of the mucinase in such methods, glycoproteins enriched, isolated, detected, identified, and/or depleted from or in a sample may remain intact or otherwise un-cleaved by the subject mucinase.

Any convenient and appropriate strategy for employing the binding of a catalytically inactive mucinase to enrich, isolate, detect, identify, and/or deplete a glycoprotein in a sample may be utilized. For example, in some instances, a catalytically inactive mucinase, rendered catalytically inactive through modification of mucinase or through the presence of a protease inhibitor, may be attached to a solid support. Useful solid supports may vary any may include but are not limited to e.g., beads, vessel (e.g., slide, well, tube, fluid-carrying channel, etc.) surfaces, resins, membranes, matrices, etc. In some instances, a sample may be contacted with a solid support having attached thereto a catalytically inactive mucinase or a mucinase rendered catalytically inactive (e.g., through the presence of a protease inhibitor) and one or more glycoproteins may be retained due to a binding interaction between the mucinase and the one or more glycoproteins. In some instances, a solid-support-attached mucinase may be configured within a vessel, such as a tube or column, including but not limited to e.g., a vessel having openings at opposite ends such that fluid may flow through the vessel thereby contacting the solid support. Various other configurations may be employed.

Where utilized, any convenient and appropriate method of mass spectrometry may be employed in analyzing samples or components thereof produced in the methods of the present disclosure. Mass spectrometry (MS) is a well-developed method for determining the characteristics of proteins including primary amino acid sequence and modifications (e.g., glycosylation). In MS-based methods, a sample (which may be solid, liquid, or gas) is ionized; the ions are separated according to their mass-to-charge ratio, e.g. by=Orbitrap, FTICR, linear ion trap, and time of flight (TOF), etc.; the ions are dynamically detected by a mechanism capable of detecting energetic charged particles, and the signal is processed into the spectra of the masses of the particles of that sample. In some instances, tandem mass spectrometry (MS/MS or MS²) may be employed, for example, to determine the sequences of peptides separated by MS.

First, all intact ions are measured in a full mass spectrum, or MS1, by the mass analyzer. Then, selected ions (usually, the most abundant ions) are subjected to fragmentation by higher-energy collision induced dissociation (HCD), collision-induced dissociation (CID), electron capture dissociation (ECD), electron transfer dissociation (ETD), infrared multiphoton dissociation (IRMPD), blackbody infrared radiative dissociation (BIRD), electron-detachment dissociation (EDD), surface-induced dissociation (SID), etc. These ions are funneled into a mass analyzer (Ion trap or Orbitrap, for instance) for measurement of the resulting fragments, which then allows for peptide sequencing and/or modification analysis.

For example, a sample, e.g. a mucinase treated sample of the present disclosure, may be applied to an LTQ ion trap mass spectrometer equipped with a Fortis tip mounted nano-electrospray ion source. In some instances, this first MS scan is followed by one or more data-dependent scans of the most abundant ions observed in the first full MS scan. Tandem MS can also be done in a single mass analyzer over time, as in a quadrupole ion trap. In some instances, MS is combined with other technologies, e.g. multiple reaction monitoring (MRM) is coupled with stable isotope dilution (SAD) mass spectrometry (MS), which allowed quantitative assays for peptides to be performed with minimum restrictions and the ease of assembling multiple peptide detections in a single measurement. In some instances, tandem mass tag (TMT) MS may be employed for quantitative analysis. Other methods for detecting peptides in a sample by MS and measuring the abundance of peptides in a sample are well known in the art; see, e.g. the teachings in US 2010/0163721, the full disclosure of which is incorporated herein by reference.

Analysis, including MS analysis, may provide various information in the methods of the present disclosure. For example, in some instances, analysis may provide the primary amino acid sequence, or a portion thereof, of a glycopeptide produced in a method of the present disclosure. In some instances, analysis may identify one or more glycans and glycosites of a glycopeptide produced in a method of the present disclosure. In some instances, analysis may provide a combination of such information, including but not limited to e.g., combinations of peptide sequence information, glycan, and glycosite information. In some instances, such information may be provided for a single glycopeptide or glycopeptides of a single fragmented mucin-domain glycoprotein. In some instances, such information may be provided for a plurality of glycopeptides, including a plurality glycopeptides of a single fragmented mucin-domain glycoprotein or a plurality glycopeptides of a plurality of fragmented mucin-domain glycoproteins. Any combination of MS-derivable data may be produced in the analyses of the present methods, including but not limited to e.g., combinations of those data described herein.

Methods of the present disclosure may or may not include one or more steps to process glycoproteins in addition to mucinase digestion. For example, in some instances, the methods may or may not include one or more protein digestion steps, e.g., employing a general protease such as trypsin. In some instances, the methods may or may not include one or more general deglycosylation steps, e.g., to generally release glycans from glycosylated proteins. Various useful and convenient deglycosylases may be employed, including but not limited to e.g., commercially available deglycosylation mixes, PNGase F, and the like. In some instances, a method of present disclosure may include mucinase digestion, e.g., using StcE, as the sole glycoprotein processing step or the sole glycoprotein digestion step of the method.

Following production of glycopeptides through the cleavage of mucin-domain glycoproteins using a mucin-specific protease such as StcE, further analysis may be performed for a variety of reasons. For example, as summarized above, analysis may provide for detection of one or more mucin-domain glycoproteins in the sample. For example, analysis may provide detection of the presence and/or absence of one or more known or unknown mucin-domain glycoproteins and/or glycopeptides. In some instances, analysis may provide for identification of one or more mucin-domain glycoproteins in the sample. For example, analysis may provide identification of the identity of one or more unknown mucin-domain glycoproteins and/or glycopeptides. In some instances, analysis may provide for the detection or identification of one or more glycopeptides generated from a mucin-domain glycoprotein (referred to as mucin-domain cleaved glycopeptides) in the sample. In some instances, analysis may provide for the production of a glycosignature of one or more glycoproteins, or a population of glycoproteins or glycopeptides, in the sample. Glycoprotein signatures may be derived de novo or may be based on a comparison, e.g., to a control or other reference.

In one embodiment, the present disclosure provides a method for the selective cleavage of mucin-domain glycoproteins and subsequent glycomapping using recombinant forms of the bacterial enzyme StcE or analogs (variants) of the bacterial enzyme StcE. In some embodiments, the method comprises (a) contacting a sample containing mucin-domain glycoproteins with a mucin-specific protease wherein the protease selectively cleaves the protein backbone thereby releasing glycosylated peptide fragments containing various glycans, (b) collecting the glycosylated peptide fragments containing various glycans separately from remainder, (c) analyzing glycopeptide fragments or (c′) releasing glycans from the glycosylated peptide fragments and analyzing released glycans and peptide fragments separately; and (d) comparing analyzed peptides, glycans, and/or glycopeptides with a control sample to obtain a glycosignature from the glycoprotein.

In some embodiments, the analogs (variants) of the bacterial enzyme StcE employed in the herein described methods comprise an amino acid sequence having at least about 70% identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity) to SEQ ID NO:1. In some instances, the StcE employed may be a variant of the StcE of SEQ ID NO:1, including where the StcE variant has less than 100% sequence identity with SEQ ID NO:1, including less than 99%, 98%, 97%, 96%, 95%, etc.

In other embodiments, the analogs (variants) of the bacterial enzyme StcE are encoded from isolated polynucleotides comprising a nucleic acid sequence having at least about 70% identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity) to SEQ ID NO:2.

In some embodiments, the analogs (variants) of the mucinase employed in the herein described methods may have an amino acid sequence having at least about 70% identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity) to any one of SEQ ID NOs:1 and 17-24.

In some embodiments, the nucleic acid sequence is codon-optimized to increase expression of the analog compared to expression from a nucleic acid sequence that is not codon-optimized. In some embodiments, the nucleic acid sequence is codon-optimized to increase expression in a particular cell type.

In another aspect, the present disclosure provides a cell that comprises a polynucleotide disclosed herein, comprising a nucleic acid sequence having at least about 70% identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity) to SEQ ID NO:2. The cell of interest can be a cell from any organism, e.g., a bacterial cell, an archaeal cell, a cell of a single-cell eukaryotic organism, a fungal cell (e.g., yeast cell, etc.), an animal cell, a cell from an invertebrate animal (e.g., fruit fly, cnidarian, echinoderm, nematode, etc.), a cell from a vertebrate animal (e.g., fish, amphibian, reptile, bird, rodent, mammal, etc.), a cell from a mammal, a cell from a mouse, a cell from a rat, a cell from a non-human primate, a cell from a human, a cell from a healthy human, a cell from a human patient, etc. In some embodiments, the cell is from a human cancer patient, or a human patient having an immune, an autoimmune, or an inflammatory disease. The cell can also be obtained from or derived from an in vivo or an animal model (e.g., an in vivo or animal model of cancer, or a model of an inflammatory disease). For instance, the cell can be obtained from or derived from a patient-derived xenograft model. The cell can be in vivo or in vitro.

Any type of cell may be of interest, such as a stem cell, e.g., embryonic stem cell, induced pluripotent stem cell, adult stem cell, e.g., mesenchymal stem cell, neural stem cell, hematopoietic stem cell, organ stem cell, a progenitor cell, a somatic cell, e.g., fibroblast, hepatocyte, heart cell, liver cell, pancreatic cell, muscle cell, skin cell, blood cell, neural cell e.g., a central nervous system cell, peripheral nervous system cell, neuron, brain cell, or spinal cord cell), immune cell, and any other cell of the body, e.g., human or animal body. The cells can be primary cells or primary cell cultures derived from a subject, e.g., an animal subject or a human subject, and allowed to grow in vitro for a limited number of passages. In some embodiments, the cells are disease cells or derived from a subject with a disease. For instance, the cells can be cancer or tumor cells or inflamed immune cells. The cells can also be immortalized cells (e.g., cell lines), for instance, from a cancer cell line. A cell of interest can also be a transplanted cell (e.g., a human cell that is transplanted into another animal such as a mouse, or a human cell contained within or derived from an organoid or organ that is transplanted into another animal such as a mouse).

Cells of interest can be harvested from a subject by any standard method. For instance, cells from tissues, such as skin, muscle, bone marrow, spleen, liver, kidney, pancreas, lung, intestine, stomach, etc., can be harvested by a tissue biopsy or a fine needle aspirate. Blood cells and/or immune cells can be isolated from whole blood, plasma or serum. In some cases, suitable primary cells include peripheral blood mononuclear cells (PBMC), peripheral blood lymphocytes (PBL), and other blood cell subsets such as, but not limited to, T cell, a natural killer cell, a monocyte, a natural killer T cell, a monocyte-precursor cell, a hematopoietic stem cell or a non-pluripotent stem cell.

Cells of interest with relevance to the herein described methods also include de-mucinated cells. As used herein, the term “de-mucinated cells”, and similar terms, will generally refer to cells that have been treated with or otherwise subjected to a mucin-specific protease. As such, de-mucinated cells will generally be substantially devoid of uncleaved mucin-domain glycoproteins. Such cells may be useful for various purpose, including but not limited to e.g., comparison to a corresponding cell population that has not been de-mucinated (i.e., non-de-mucinated cells or a population thereof), comparison to a fraction containing the glycopeptides cleaved from the de-mucinated cells (e.g., a supernatant obtained from or isolated from the de-mucinated cells), as a negative control for investigating a protein known or suspected of binding a mucin-domain glycoprotein, or the like.

Any sample may be de-mucinated as desired through contact with a mucin-specific protease under conditions sufficient for mucin-specific protease-mediated cleavage of mucin-domain glycoproteins within the sample. Samples that may be de-mucinated may be cellular samples or acellular samples. De-mucination of a sample will produce cleaved glycoproteins or glycopeptides and byproduct of the cleavage reaction. For example, where glycoproteins are attached to some proteinaceous and non-proteinaceous member, such as a matrix or membrane, de-mucinating the sample will generate cleaved glycoproteins and the proteinaceous and non-proteinaceous member with the previously attached glycoproteins removed. For example, in the case of a cellular sample, de-mucinating a cellular sample by contacting the sample with a mucin-specific protease under conditions sufficient for mucin-specific protease-mediated cleavage, will generate glycoproteins and byproduct that includes de-mucinated cells.

In some embodiments, the present disclosure provides a method for detecting a disease condition in a subject that is characterized by aberrant glycosylation and is associated with a particular glycosignature by carrying out glycomapping and determining glycosignatures in biological samples of the subject. Mucin-domain specific proteolysis will facilitate discovery of unique glycosignatures where mucin-domain glycoproteins, e.g., in the host or in an infectious agent, make the host more susceptible to a disease, cause the development of a disease and/or contribute to disease progression. Likewise, the existence of unique glycosignatures may also make a host less susceptible to a disease, prevent the development of a disease and/or prevent disease progression.

Such disease conditions can be any disease where mucin-domain glycoproteins, for example, as part of the host or as part of an infectious agent, play any role in causing the disease, progressing the disease, or spreading the disease. Diseases may include, for example, cancer, viral infections, bacterial infections, inflammatory bowel disease, infective endocarditis, autoimmune diseases.

In some instances, a method for detecting a condition or disease characterized by aberrant glycosylation in a subject may include determining a mucin-domain cleaved glycosignature from a biological sample from said subject according to the methods described herein. Such methods may include comparing a mucin-domain cleaved glycosignature to a healthy reference or control mucin-domain cleaved glycosignature. An appropriate control may include but is not limited to e.g., a sample (e.g., a cellular sample) obtained from a subject known not to have the subject condition that is prepared and analyzed in a manner corresponding to the experimental sample. An appropriate reference may include but is not limited to e.g., a reference data set obtained from a subject or a sample known not to have the subject condition, where the reference data set was obtained, prepared and analyzed in a manner corresponding to the experimental sample. Such comparisons, e.g., with control and/or reference samples and/or data, as performed in the methods may allow one to detect the condition or disease in the subject, including e.g., where the disease is cancer, viral infections, bacterial infections, inflammatory bowel disease, infective endocarditis, autoimmune diseases, or a related condition.

Methods of the present disclosure also include methods of treating a subject for a condition where the method includes performing, or having performed, a method for detecting a condition characterized by aberrant glycosylation in to detect whether the subject has the condition, and then treating the subject when the condition is detected. As an example, such methods may include performing, or having performed, a method as described herein to detect whether a subject has a cancer characterized by aberrant glycosylation, and then treating the subject when the subject is identified as having the cancer characterized by aberrant glycosylation.

In some instances, treating a subject may include treating a subject with a conventional cancer therapy (such as chemotherapy or radiation) and/or treating the subject with a mucin-domain directed therapy. Mucin-domain directed therapies will vary and will generally include those therapies employing therapeutics that bind to or otherwise target or abrogate the signaling of mucin-domain glycoproteins. Non-limiting examples of mucin-domain directed therapies include but are not limited to e.g., mucin-domain glycoprotein-specific antibody therapies, mucin-domain glycoprotein-specific chimeric antigen receptor (CAR) therapies, anti-mucin vaccine therapies, mucin inhibitor therapies, and the like.

Mucin-domain glycoprotein-specific antibody therapies will vary and will generally include administering to the subject an effective amount of a therapeutic antibody specific for a mucin-domain glycoprotein or a variant or mutant thereof. A non-limiting example of such mucin-domain glycoprotein-specific antibody therapies include MUC1-specific antibody therapies. Antibodies have been generated targeting the shed MUC1-N subunit and against the cell-bound MUC1-C subunit in the region that interacts with MUC1-N. Antibodies have also been generated against MUC4 and the overexpression of MUC16 in ovarian cancer cells represents a target for therapeutic antibodies. An antibody against the MUC16 tandem repeats has been conjugated to the cytotoxic auristatins and shown to be active against human OVCAR-3 ovarian tumor xenografts. In addition, the interaction between MUC16 and mesothelin has been blocked with an antibody against the MUC16 binding domain on mesothelin to produce an alternative therapeutic approach.

Mucin-domain glycoprotein-specific chimeric antigen receptor (CAR) therapies will generally involve the production and administration of CAR T cell expressing a CAR specific for a mucin-domain glycoprotein, such as but not limited to e.g., a mucin-domain glycoprotein described herein.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled (e.g., as described in PCT publication no. WO 2014/127261 A1 and US Patent Application No. 2015/0368342 A1, the disclosures of which are incorporated herein by reference in their entirety). CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety. CARs also include the anti-CD19-4-1BB-CD3ζ CAR expressed by lentivirus loaded CTL019 (Tisagenlecleucel-T) CAR-T cells as commercialized by Novartis (Basel, Switzerland) and the anti-CD19-CD28-CD3ζ CAR of Axicabtagene Ciloleucel as commercialized by Kite Pharma, Inc. (Santa Monica, Calif.).

Such commercial CARs may be modified, e.g., by replacing the antigen binding domain with an anti-mucin-domain glycoprotein domain to readily produce an anti-mucin-domain CAR T cell therapy. Useful anti-mucin-domain glycoprotein CAR T cell therapies also include but are not limited to those targeting MUC1 as employed in ClinicalTrials(dot)gov Identifier: NCT03633773 “Safety and Efficacy Evaluation of MUC-1 CART in the Treatment of Intrahepatic Cholangiocarcinoma” and NCT02587689 “Phase I/II Study of Anti-Mucin1 (MUC1) CAR T Cells for Patients With MUC1+Advanced Refractory Solid Tumor”, and the like.

Anti-mucin vaccine therapies will generally involve the administration of an antigen to a subject to induce an immune response to a mucin-domain glycoprotein, including but not limited to where the mucin-domain glycoprotein is a mucin-domain glycoprotein described herein. Useful anti-mucin vaccine therapies include but are not limited to e.g., vaccines against MUC1, including e.g., the BLP25 liposome vaccine (L-BLP25, also known as stimuvax; Oncothyreon, Merck KGaA, EMD Serono; a liposome-based vaccine designed to induce an immune response against the MUC1 tandem repeats) and TG4010 (Transgene; a modified vaccinia virus expressing MUC1 and IL-2).

Mucin inhibitor therapies will generally involve peptide or non-peptide small molecule inhibitors of mucin-domain glycoprotein binding/interaction and/or signaling. Useful mucin inhibitor therapies include those directed to essentially any mucin-domain glycoprotein, including but not limited to those described herein. As a non-limiting example, a useful mucin inhibitor includes a peptide derived from the MUC1-C cytoplasmic domain (designated PMIP) has been used as a decoy for binding to β-catenin and a substrate for EGFR and SRC phosphorylation (Bitler et al., Clin Cancer Res. 2009; 15:100-109). Another strategy involves direct targeting of the MUC1-C CQC motif with a peptide (GO-201) that inhibits MUC1-C oligomerization (Raina et al., Cancer Res. 2009; 69:5133-5141).

The above listed examples of mucin-domain directed therapies should not be construed as limiting and essentially any appropriate therapy resulting in the desired therapeutic outcome in subjects identified as described may be employed.

### Kits and Compositions

Aspects of the present disclosure also include compositions and kits and, in some instances, devices, for use therewith or therein. The compositions and kits may include, e.g., one or more of any of the reaction mixture components described above with respect to the subject methods.

In another aspect, the present disclosure provides a kit that is useful in the selective cleavage of mucin-domain glycoproteins and glycomapping. In some instances, a kit may include a StcE protein or other mucinasse. Components of the subject kits may be provided in various forms, including e.g., liquid or dry forms. In some instances, a StcE or variant thereof in a subject kit may be provided in lyophilized form.

In some embodiments, the kit comprises a polynucleotide disclosed herein (e.g., a polynucleotide encoding a StcE, Pic, ZmpC, BT4244, AM0627, AM0908, AM1514, SmEnhancin, VIBHAR2194, an analog, mutant, or a derivative thereof) and/or a cell disclosed herein (e.g., a cell comprising a polynucleotide encoding a StcE, Pic, ZmpC, BT4244, AM0627, AM0908, AM1514, SmEnhancin, VIBHAR2194, an analog, mutant, or a derivative thereof), and/or the purified mucinase, such as, StcE, Pic, ZmpC, BT4244, AM0627, AM0908, AM1514, VIBHAR2194, an analog, mutant, or a derivative thereof. Polynucleotides may be provided in various form. For example, polynucleotides may be provided as DNA or RNA and may, in some instances, be included as a vector including RNA or DNA vectors. In some instances, a DNA encoding StcE or a variant thereof is provided as a plasmid containing the encoding sequence.

In some embodiments, the kit further comprises one or more reagents. The reagents can be used, as non-limiting examples, to introduce a polynucleotide into the cell, to express an analog of StcE in the cell, to deglycosylate glycoproteins of a sample, to digest proteins of a sample, to enrich for or isolate a component of a sample, and the like. As such, various reagents may be included in the subject kits including but not limited to e.g., a transfection reagent, a deglycosylating enzyme (e.g., PNGase F), a protease (e.g., trypsin), etc.

In some instances, kits of the present disclosure may include one or more buffers or dry compositions for producing a buffer. For example, in some instances, a buffer may be provided for performing a mucinase cleavage reaction. Such buffers will vary and will generally be configured such that the mucin-specific protease, e.g., StcE or variant thereof, is active in the buffer.

In other embodiments, the kit comprises a mucin-specific bait protein that is conjugated to a solid-phase matrix such as beads, wherein the mucin-specific bait protein is StcE, a recombinant polypeptide comprising a sequence that has at least 90% sequence identity to any one of SEQ ID NOs:1 and 17-24, or a polynucleotide comprising a sequence that has at least 70% sequence identity to SEQ ID NO:2.

In some instances, a kit of the present disclosure may include one or more devices, e.g., a device for performing one or more steps of a method as described herein. For example, in some instances a subject kit may include a purification device for isolating and/or enriching proteins or peptides of the sample. Such devices will vary may include but are not limited to e.g., protein purification columns (e.g., a protein purification resin column, a protein purification spin column, etc.), and the like).

In some embodiments, the kit further comprises instructions for use. The instructions pertain to, as non-limiting examples, introducing a polynucleotide into the cell, expressing an analog of StcE in the cell, purifying mucins from protein compositions, carrying out the selective analysis of mucin-domain glycoproteins, and so forth.

The instructions are generally recorded on a suitable recording medium. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, Hard Disk Drive (HDD) etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXPERIMENTAL PROCEDURES

The following methods and materials were used in the examples that are described further below.

### Expression and Purification of StcE and E447D

E447D was generated using the Q5 Site-Directed Mutagenesis Kit (New England Biolabs), with primers 5′-3′ (SEQ ID NO:9) and 5′-ACTCATTCCCCAATGTGG-3′ (SEQ ID NO:10). E. coli BL21(DE3) were transformed with pET28b-StcE-435-NHis or pET28b-StcE-E447D-Δ35-NHis and grown at 37° C. until an optical density of 0.6-0.8 was reached. The culture was then induced with 0.3 mM IPTG and incubated at 20° C. overnight. Cells were lysed in 20 mM HEPES pH 7.5, 500 mM NaCl using a probe tip sonicator. Lysates were applied to HisTrap HP columns (GE Healthcare Life Sciences) using a GE ÄKTA Pure FPLC. After washing with 20 column volumes of lysis buffer+20 mM imidazole, elution was performed using a 15 mM linear gradient from 20 mM imidazole to 250 mM imidazole. Pooled fractions for each enzyme were concentrated using Amicon Ultra 30 kDa MWCO filters (Millipore Sigma), then snap frozen in liquid nitrogen and stored at −80° C.

### In Vitro StcE Activity Assays

Recombinant and purified mucins were purchased from Molecular Innovations (C11NH), and R&D Systems (MUC16, podocalyxin, CD43, PSGL-1, Syncam-1, and CD45). To test StcE's activity against mucin-like glycoproteins and non-mucins, reaction conditions were as follows: 1:10 enzyme:substrate (E:S) ratio, total volume of 15 μL, buffer 0.1% Protease Max in 50 mM ammonium bicarbonate, 3 hours at 37° C. A portion of each condition (0.5 μg) was loaded onto 10% Criterion™ XT Bis-Tris precast gels (Bio-Rad) and run with XT-MES (Bio-Rad) at 180 V for 1 h. Each gel was stained with silver stain or Pro-Q Emerald 300 Glycoprotein Gel and Blot Stain Kit® (Thermo Fisher Scientific), according to manufacturer's instructions. Deglycosylation of rhMUC16 was performed according to manufacturer's instructions (Deglycosylation Mix, Promega). Mucin mimetic copolymer consisting of 50% GalNAc-Ser and 50% Lys was synthesized as previously described (Kramer et al., Proc. Natl. Acad. Sci. (2015) 112, 12574-12579. Both deglycosylated polymer and untreated polymer were subjected to StcE cleavage and gel staining as described above for recombinant protein substrates. For peptide cleavage assays, four synthetic peptides were subjected to StcE treatment. The peptide sequences were as follows: RPPI(T-GalNAc)QSSL (SEQ ID NO:11), IPV(S-GalNAc)SHNSL (SEQ ID NO:12), IPVS(S-GalNAc-Galactose)SHNSL (SEQ ID NO:13), and DRV(Y-Phosphate)IHPF (SEQ ID NO:14). StcE was added (1:10 E:S) to 50 μL of a 500 fmol/μL solution containing all four peptides for 3 hours at 37° C. The solution was subjected to a C18 cleanup and MS analysis as described below.

### StcE Digests and Mass Spectrometry Sample Preparation

A sample (5 μg) of each recombinant glycoprotein was digested with StcE in a 1:10 E:S ratio, in a total volume of 15 μL of buffer (0.1% Protease Max in 50 mM ammonium bicarbonate) for 3 h at 37° C. Control proteins were incubated at 37° C. for 3 h in a solution containing buffer only. Afterward, the volume was increased to 19 μL with buffer. For deglycosylated samples, 0.5 μL of 10× Deglycosylation Reaction Buffer (Promega) and 0.5 μL of Protein Deglycosylation Mix (Promega) were added to give a total volume of 20 μL. For PNGaseF treated samples, 1 μL of PNGaseF (Promega) was added to 99 μL of 50 mM ammonium bicarbonate, and 1 μL of this reaction was added to each StcE reaction vial. Deglycosylation reactions were incubated overnight (12-16 h) at 37° C. Reduction and alkylation were performed according to ProteaseMax (Promega) protocols. Briefly, the solution was diluted to 93.5 μL with 50 mM ammonium bicarbonate. Then, 1 μL of 0.5 M DTT was added and the samples were incubated at 56° C. for 20 min, followed by the addition of 2.7 μL of 0.55 M iodoacetamide at room temperature for 15 min in the dark. Digestion was completed by adding sequencing-grade trypsin (Promega) in a 1:20 enzyme:protein ratio for 8 h at 37° C. and quenched by adding 0.3 μL of glacial acetic acid. C18 clean-up was performed using SPEC tips (Agilent). Each tip was wet with 200 μL of methanol three times, followed by three 200 μL rinses of buffer A (5% formic acid in water). The samples were diluted to 200 μL in buffer A and loaded through the column 5-6 times, then rinsed three times with buffer A. Finally, the samples were eluted with three rinses of 100 μL buffer B (5% formic acid, 80% acetonitrile) and dried by speedvac.

### Mass Spectrometry

Samples were reconstituted in 10 μL 0.1% formic acid (Fisher Scientific) containing 25 fmol/μL angiotensin (Millipore Sigma) and vasoactive peptide (Anaspec). Samples were analyzed by online nanoflow LC-MS/MS using an Orbitrap Fusion Tribrid mass spectrometer (Thermo Fisher Scientific) coupled to a Dionex Ultimate 3000 HPLC (Thermo Fisher Scientific). A portion of the sample (4 μL) was loaded via autosampler onto a 20 μL sample loop and injected at 0.3 μL/min onto a 75 μm×150 mm EASY-Spray column (Thermo Fisher Scientific) containing 2 μm C18 beads. The column was held at 40° C. using a column heater in the EASY-Spray ionization source (Thermo Fisher Scientific). The samples were eluted at 0.3 μL/min using a 90 minute gradient and a 185 minute instrument method. Solvent A was comprised of 0.1% formic acid in water, whereas Solvent B was 0.1% formic acid in acetonitrile. The gradient profile was as follows (min:% B) 0:3, 3:3, 93:35, 103:42, 104:98, 109:98, 110:3, 185:3. The instrument method used an MS1 resolution of 60,000 at FWHM 400 m/z, an AGC target of 3e5, and a mass range from 300 to 1,500 m/z. Dynamic exclusion was enabled with a repeat count of 3, repeat duration of 10 s, exclusion duration of 10 s. Only charge states 2-6 were selected for fragmentation. MS2s were generated at top speed for 3 s. HCD was performed on all selected precursor masses with the following parameters: isolation window of 2 m/z, 28-30% collision energy, ion trap or orbitrap (resolution of 30,000) detection, and an AGC target of 1e4 ions. ETD was performed if (a) the precursor mass was between 300-1000 m/z and (b) 3 of 7 glyco-fingerprint ions (126.055, 138.055, 144.07, 168.065, 186.076, 204.086, 274.092, 292.103) were present at +/−0.5 m/z and greater than 5% relative intensity. ETD parameters were as follows: calibrated charge-dependent ETD times, 2e5 reagent target, precursor AGC target 1e4.

### Mass Spectrometry Data Analysis

Raw files were searched using Byonic by ProteinMetrics against the Uniprot human proteome (downloaded Jun. 26, 2016) and/or directed databases containing the recombinant protein of interest. Search parameters included semi-specific cleavage specificity at the C-terminal site of R and K. Mass tolerance was set at 10 ppm for MS1s, 0.35 for MS2s. Methionine oxidation (common 2), asparagine deamidation (common 2), and N-term acetylation (rare 1) were set as variable modifications with a total common max of 3, rare max of 1. O-glycans were also set as variable modifications (common 2), using the “O-glycan 6 most common” database. Cysteine carbaminomethylation was set as a fixed modification. Peptide hits were filtered using a 1% FDR. All peptides were manually validated and/or sequenced using Xcalibur software (Thermo Fisher Scientific). HCD was used to confirm that the peptides were glycosylated, whereas ETD spectra were used for site-localization of glycosylation sites.

### Cell Culture

Cells were grown in T75 flasks (Thermo Fisher Scientific) and maintained at 37° C. and 5% CO₂. BT-20, HeLa, and MDA-MB-453 cells were cultured in DMEM supplemented with 10% fetal bovine serum (FBS) and 1% penicillin/streptomycin. SKBR3, K562, and ZR-75-1 cells were cultured in RPMI supplemented with 10% FBS and 1% penicillin/streptomycin. Ldl-D CHO cells were cultured in 1:1 DMEM/F12 with 3% FBS and 1% penicillin/streptomycin. MCF10A MUC1ΔCT cells were cultured in phenol red free 1:1 DMEM:F12 supplemented with 5% New Zealand horse serum (Thermo Fisher Scientific), 20 ng/mL epidermal growth factor (Peprotech), 0.5 μg/mL hydrocortisone (Millipore Sigma), 100 ng/mL cholera toxin (Millipore Sigma), 10 μg/mL insulin (Millipore Sigma), and 1% penicillin/streptomycin. MUC1ΔCT was induced with 200 ng/mL doxycycline for 24 h.

### Cell Viability Assay

HeLa cells and K562 cells were seeded in 48-well plates at 10,000 cells per well in 500 μL of complete media. After growth overnight (24 h), StcE was added at 500, 50, 5, 0.5, 0.05, 0.005, and 0 nM. At t=27, 48, 76, and 101 h post treatment, viability was measured using PrestoBlue (Thermo Fisher Scientific) and a bottom read fluorescence plate reader, according to manufacturer instructions.

### Flow Cytometry and Western Blotting of StcE-Treated Cells

Cells were treated with StcE or E447D when plated or after lifting with enzyme-free cell dissociation buffer (Thermo Fisher Scientific). Typical treatment conditions were 5 μg of StcE per 1 million cells in 1 mL of complete media or Hank's Buffered Salt Solution (HBSS) for two hours at 37° C. After treatment, cells were washed with PBS or HBSS. For flow cytometry, cells were resuspended in cold PBS with 0.5% bovine serum albumin and transferred to a 96-well V-bottom plate. Cells were then resuspended in the probe of interest. Flow cytometry data was analyzed using FlowJo v. 10.0 (Tree Star). For Western blots, supernatants post-treatment (1 mL volumes) were collected into tubes containing 75 μL of 0.5 M EDTA to quench the reaction, then snap frozen in liquid nitrogen and lyophilized to dryness. Post-treatment cells were washed with enzyme-free cell dissociation buffer, which contains EDTA, to quench the reaction. Cells were then washed two times with PBS, pelleted, and lysed with sample buffer (1× NuPAGE LDS Sample Buffer (Thermo Fisher Scientific) supplemented with 25 mM DTT). Genomic DNA was sheared via probe tip sonication. Lyophilized supernatants were brought up in sample buffer. Both cell lysates and supernatants were boiled for 5 min. at 95° C., spun at 14,000×g for 2 min., and 30 μL of each was loaded into an 18-well 4-12% Criterion™ XT Bis-Tris precast gel (Bio-Rad). The gel was run with XT-MOPS (Bio-Rad) at 180 V for 1 h. Proteins were transferred to 0.2 μm nitrocellulose using the Trans-Blot® Turbo™ Transfer System (Bio-Rad), at 2.5 A constant for 15 min. Total protein was quantified using REVERT stain (LI-COR Biosciences) or Ponceau-S stain (Millipore Sigma).

### Antibodies for Flow Cytometry

Anti-MUC16 antibody [X75] (Abcam) and anti-MUC1 (VU4H5) Mouse mAb #4538 (Cell Signaling Technology) were used according to manufacturer recommendations to stain for cell surface MUC1 and MUC16, respectively. Trastuzumab was conjugated to Alexa Fluor-647 using Alexa Fluor® 647 Antibody Labeling Kit (Thermo Fisher Scientific); staining for HER2 was performed at 1.2 μg/mL. Human recombinant Siglec-7-Fc chimera or Siglec-9-Fc chimera (2 μg/mL, R&D Biosystems) were precomplexed with 4 μg/mL fluorescently labeled anti-human antibody prior to use. Anti-His-FITC and Mouse IgG1-FITC (Miltenyi Biotech) were used according to manufacturer recommendations to stain for surface resident StcE and E447D. Primary and secondary antibody staining was performed for 30 min at 4° C. Two or three washes between primary and secondary staining were performed with 0.5% BSA in PBS. Secondary antibodies (Jackson ImmunoResearch) were used at 4 μg/mL, and cells were washed three times after staining Live cell periodate-based labeling of sialic acids was performed as previously described (Zeng et al., Nat. Methods (2009) 6, 207-209).

### Antibodies for Western Blot

Anti-MUC16 antibody [X75] (Abcam), IRDye® 800CW Goat anti-Mouse IgG (LI-COR Biosciences), anti-MUC1 (VU4H5) Mouse mAb #4538 (Cell Signaling Technology), and anti-mouse IgG, HRP-linked Antibody (Cell Signaling Technology) were used according to manufacturer recommendations.

Enrichment of C11NH with StcE-Conjugated Beads

StcE was concentrated in a 30 kDa MWCO Amicon filter (Millipore Sigma) from 1.93 mg/mL to 7.76 mg/mL. The concentrated enzyme was added to 2.72 mg of 20 μm POROS AL beads (Thermo Fisher Scientific), followed by the addition of 0.5 μL of 80 mg/mL NaCNBH₃ (Millipore Sigma). After incubation overnight at 4° C., the beads were washed three times with water, and brought up in a final volume of 500 μL. For the pulldown, 100 μL of 0.1 mg/mL BSA, 0.5 μL of 2 mg/mL C1-INH, and 10 μL 250 mM EDTA were added to 50 μL of the bead slurry. Reaction buffer was PBS. The reaction proceeded for 3 h at room temperature with shaking. After the reaction, the beads were spun down and the supernatants were saved (“flow through”). The beads were sequentially washed once with 100 μL PBS (“wash 1”), once with 100 μL 1% Tween (“wash 2”), and once with 100 μL PBS (“wash 3”). For the elution, 32 μL 1×NuPage LDS sample buffer (Thermo Fisher Scientific) was added and the beads were boiled for 5 min. Samples were loaded onto a 10% Criterion™ XT Bis-Tris precast gel (Bio-Rad), run at 180 V for 1 h with XT-MES (BioRad), and visualized by silver stain.

### Molecular Modeling

Using the 2016 Molecular Operating Environment (MOE) software suite, the X-ray crystal structures of StcE (PDB ID: 3UJZ), astacin (PDB ID: 1QJI), and serralysin (PDB ID: 3VI1) were superimposed using the residues (HEXXHXXGXXH) of their conserved metzincin active site (Gomis-Rüth et al., J. Biol. Chem. (2009) 284, 15353-15357). The individual structures were then prepared by (a) capping any termini with acetyl or NMe groups and (b) adding unresolved atoms (side chains and hydrogens) so that each structure was at its proper valency and charge. In their cocrystal structures, the peptidomimetic/peptidic ligands bind in the active site of astacin/serralysin in similar conformations, with the ligands' P2-P1′ residues forming antiparallel β-sheets with the enzymes. These crystallographic ligands were thus used as scaffolds to construct the three different ligands used in our docking studies: Ac-PTLTH-NMe (SEQ ID NO:7), Ac-P(GalNAcα-)TLTH-NMe (SEQ ID NO:8), and Ac-P(GalNAcα-) TL(GalNAcα-)TH-NMe (SEQ ID NO:4), where Pro is the P3 residue and His is the P2′ residue. Using the Amber10:EHT forcefield, each of the three ligands underwent a brief dynamics simulation to generate a corresponding library of >15,000 conformers, with the individual conformations varying solely in the arrangement of their side chains and GalNAc moieties. Each conformer and the prepared StcE(E447D) structure underwent induced fit docking, again using the Amber10:EHT forcefield, to yield minimized ligand-enzyme complexes. Docking studies containing the normal catalytic E447 residue and/or solvent molecules did not yield reasonable or reproducible results.

StcE treatment of patient-derived CA-125 Fresh frozen ovarian cancer patient-derived ascites fluid was rapidly thawed in a room temperature water bath, then centrifuged at 500×g for 5 min. at 4° C. to remove cellular debris. A portion of clarified solution (50 (IL) was treated with 5, 0.5, 0.05, or 0.005 μg StcE for 1 h at 37° C. An aliquot of reaction solution (22.5 (IL) was removed to tubes containing 7.5 μL 4×NuPAGE LDS Sample Buffer (Thermo Fisher Scientific)+100 mM DTT, then boiled for 5 min at 95° C. to quench the reaction. Boiled samples were spun at 14000×g for 2 min., then 20 μL of each was loaded onto an 18-well 4-12% Criterion™ XT Bis-Tris precast gel (Bio-Rad), and the gel was run with XT-MOPS (Bio-Rad) at 180 V for 1 h. Proteins were transferred to 0.2 μm nitrocellulose using the Trans-Blot® Turbo™ Transfer System (Bio-Rad) at 2.5 A constant for 15 min. Total protein was quantified using REVERT stain (LI-COR Biosciences). Western blotting for MUC16 was performed using anti-MUC16 antibody [X75] (Abcam) according to manufacturer recommendations. IRDye® 800CW Goat anti-Mouse IgG (LI-COR Biosciences) was used according to manufacturer recommendations. Reactions with semi-crude Cancer Antigen 125 (Lee BioSolutions) were performed in the same manner as recombinant substrates (see above) and immunoblotted with anti-MUC16 antibody as was done for patient-derived ascites fluid.

Expression and purification of Pic, ZmpC, BT4244, BT4244 E575A, AM0627, AM0627 E326A, AM0908, AM1514, SmEnhancin, and VIBHAR2194

The gene fragments encoding Pic (SEQ ID NO: 20), ZmpC (SEQ ID NO: 21), BT4244 (SEQ ID NO: 22), AM0627 (SEQ ID NO: 23), AM0908 (SEQ ID NO: 24), AM1514 (SEQ ID NO: 25), SmEnhancin (SEQ ID NO: 26), and VIBHAR2194 (SEQ ID NO: 27) were amplified from genomic DNA. AM0627 was cloned into pET28b (Novagen), BT4244 was cloned into pRSETA (Invitrogen), Pic was cloned into pACYC184, SmEnhancin was cloned into pET28a, and the rest were cloned into pRham Chis (Lucigen). E326A and E575A were generated using the Q5 Site-Directed Mutagenesis Kit (New England Biolabs). Plasmids encoding AM0627, E326A, BT4244, E575A, and SmEnhancin were transformed into E. coli BL21(DE3) and grown at 37° C. until an optical density of 0.6-0.8 was reached. The AM0627, E326A, BT4244, and E575A cultures were then induced with 0.4 mM IPTG and incubated for an additional 3 hours at 37° C. The SmEnhancin culture was induced with 0.1 mM IPTG and grown overnight at 16° C. Plasmids encoding ZmpC, AM0908, AM1514, and VIBHAR2194 were transformed into E. coli 10G (Lucigen) and grown at 37° C. until an optical density of 0.4-0.8 was reached. Cultures were induced with 0.2% v/v rhamnose and incubated for an additional 3 hours at 37° C. Cells were lysed with xTractor buffer (Clontech) and lysates were applied to a 1 mL HisTrap HP column (GE Healthcare Life Sciences) using a GE ÄKTA Pure FPLC. Fractions containing pure protein as judged by SDS-PAGE analysis were pooled and concentrated using a 10K Amicon Ultra MWCO filter (Millipore Sigma), dialyzed into PBS, and stored at −80° C. pACYC184-Pic was transformed into E. coli DH5a, grown to OD 0.7-1, concentrated using a 50K Amicon Ultra MWCO filter (Millipore Sigma), dialyzed into PBS, and stored at −80° C.

### EXAMPLES

The following examples are put forth so as to provide those of ordinary skill in the art with a complete disclosure and description of how to make and use the present invention; they are not intended to limit the scope of what the inventors regard as their invention. Unless indicated otherwise, part are parts by weight, molecular weight is average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); and Cell and Tissue Culture: Laboratory Procedures in Biotechnology (Doyle & Griffiths, John Wiley & Sons 1998), the disclosures of which are incorporated herein by reference. Reagents, antibodies, cells, tissue samples, etc., and kits referred to in this disclosure are available from commercial vendors such, but not limited to, those vendors identified herein.

### Example 1: STCE has Peptide-, Glycan-, and Secondary Structure-Based Specificity for Mucins

The field of glycoproteomics has been almost entirely focused on N-glycosylated proteins, which have predictable glycosylation sites and structures, convenient enzymatic tools for glycan manipulation, and effective software for site and structure assignments. Mucin glycoproteins, on the other hand, defy all these conveniences.

In addition, due to the presence of tandem repeat domains, MUC1 can be >1200 amino acids long and 50% glycosylation by mass; MUC16 can exceed 22,000 residues and 85% glycosylation by mass. The high density of O-glycosylation on these tandem repeats makes them resistant to digestion by workhorse proteases such as trypsin, meaning the majority of the sequence space is often left unanalyzed in conventional methods. Systems with truncated forms of glycosylation, such as engineered “SimpleCells” lacking the 0-glycan elaboration machinery, can simplify the identification of glycosylation sites; however, in these methods functionally important glycan structures beyond the initiating 0-GalNAc are lost. In view of these realizations, the following investigations were undertaken. The work described in the following examples, along with the description of the present disclosure, provide tools with peptide-, glycan-, and secondary structure-based specificity for mucins and methods for applying such tools.

StcE and its catalytically inactive point mutant (E447D) were expressed as 98 kDa soluble N-terminal His-tagged proteins in E. coli, as previously described (see FIG. 6) (Yu et al., Structure (2012) 20, 707-717). StcE activity against a known substrate, C1 esterase inhibitor (C11NH), is detectable in a pH range of 6.1-9.0, in a temperature range of 4-55° C., in high salt and detergent, and after days of incubation at 37° C., consistent with its pathological activity in the mammalian gut.

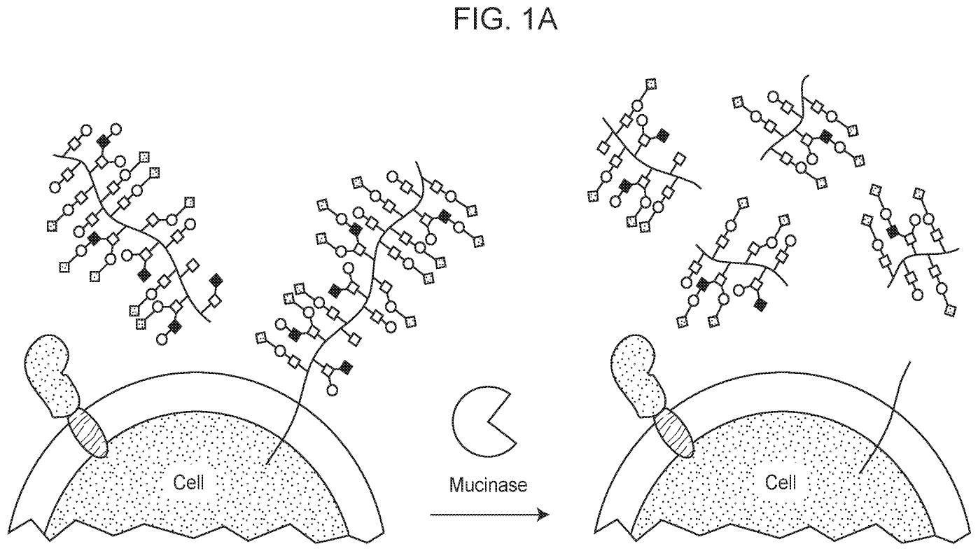

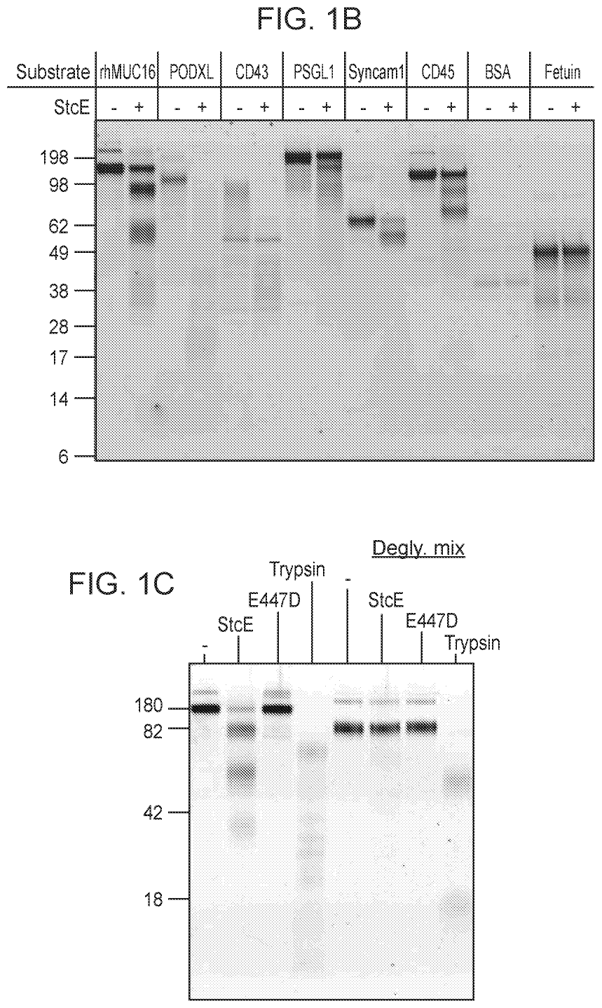

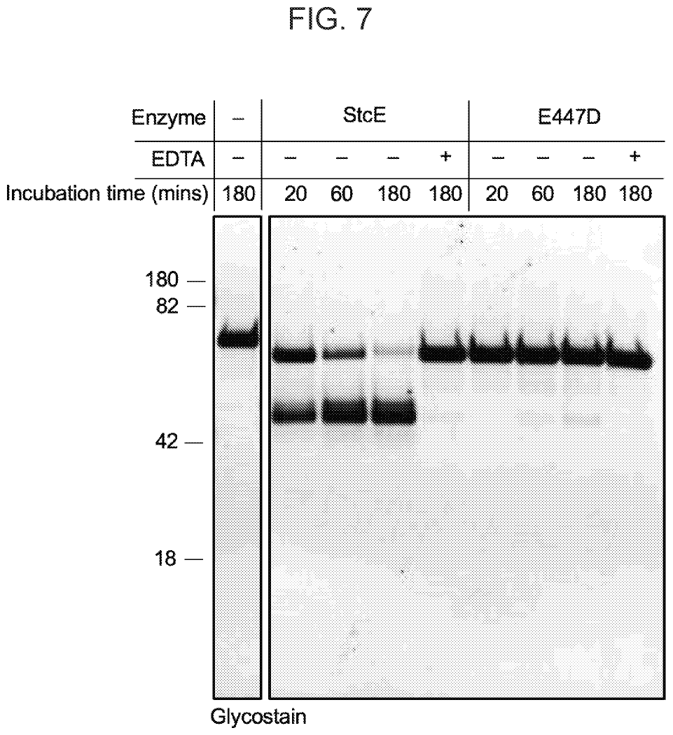

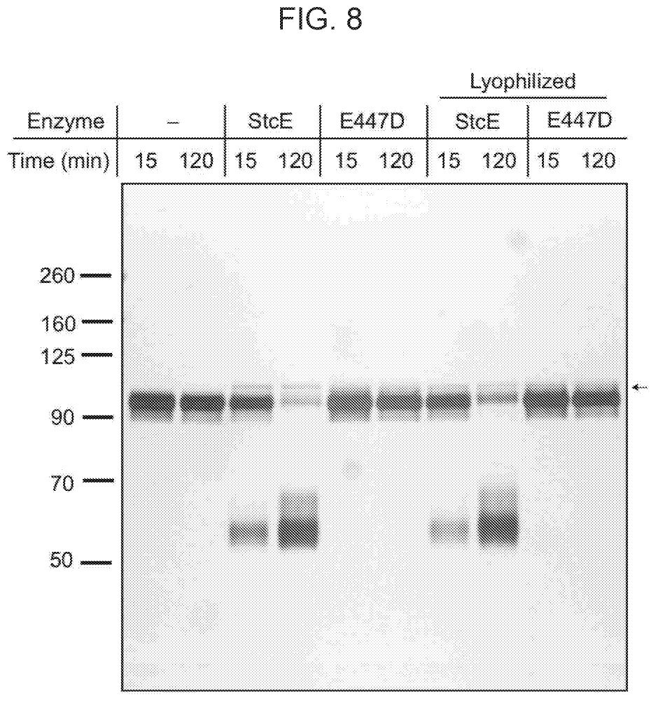

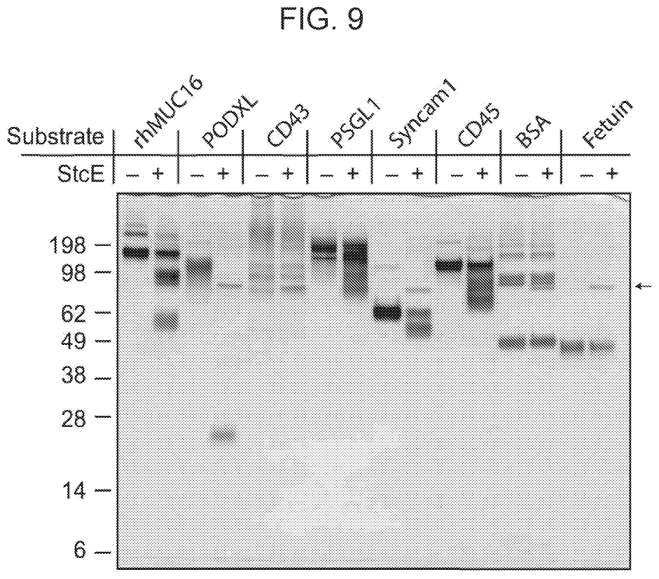

Glycan requirement for cleavage by StcE. StcE was amenable to high yield expression (80 mg/L), active against C11NH (see FIG. 7), stable to lyophilization (see FIG. 8), and operative at nanomolar concentrations in all media types tested. Next, StcE's activity on clinically relevant mucin-domain glycoproteins was assessed. StcE did not cleave glycosylated but non-mucin proteins (e.g., bovine serum albumin and fetuin), but cleaved all tested mucin-like glycoproteins (e.g., recombinant MUC16, podocalyxin, CD43, PSGL-1, Syncam-1, and CD45), as evidenced by gel shifts to lower molecular weights (glycostain and silver stain, see FIG. 1B, and FIG. 9B). Further, StcE's activity was abrogated when its substrates were enzymatically deglycosylated, indicating a glycan requirement for cleavage (FIG. 1C).

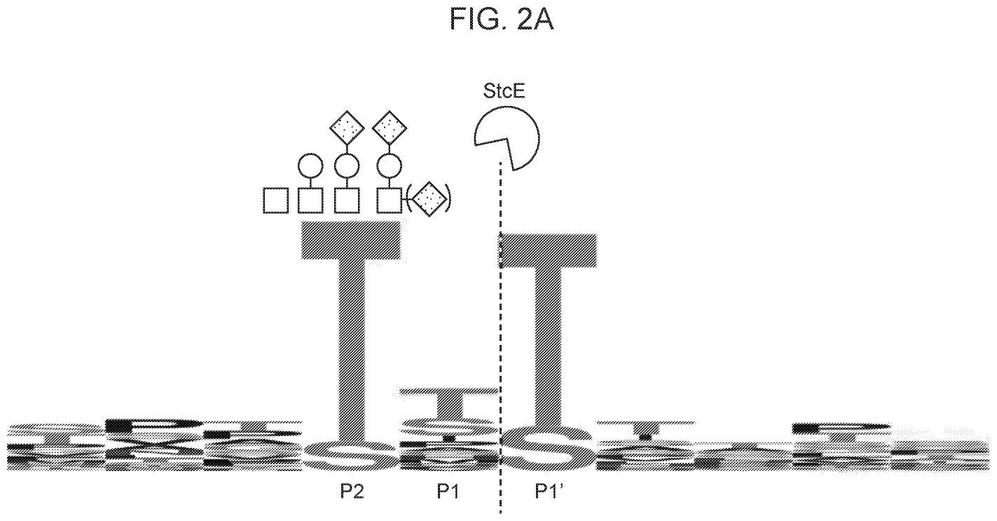

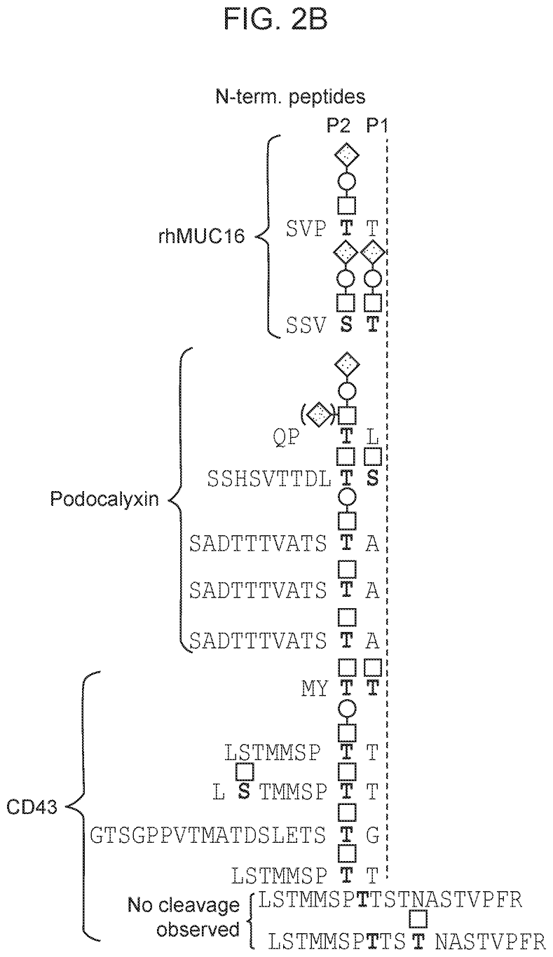

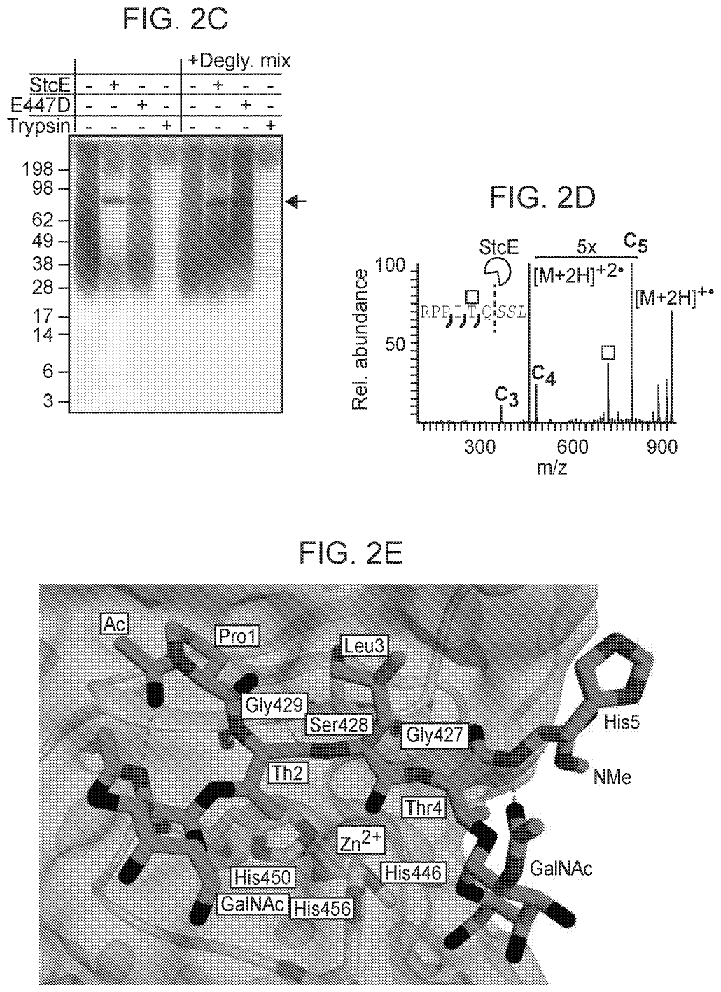

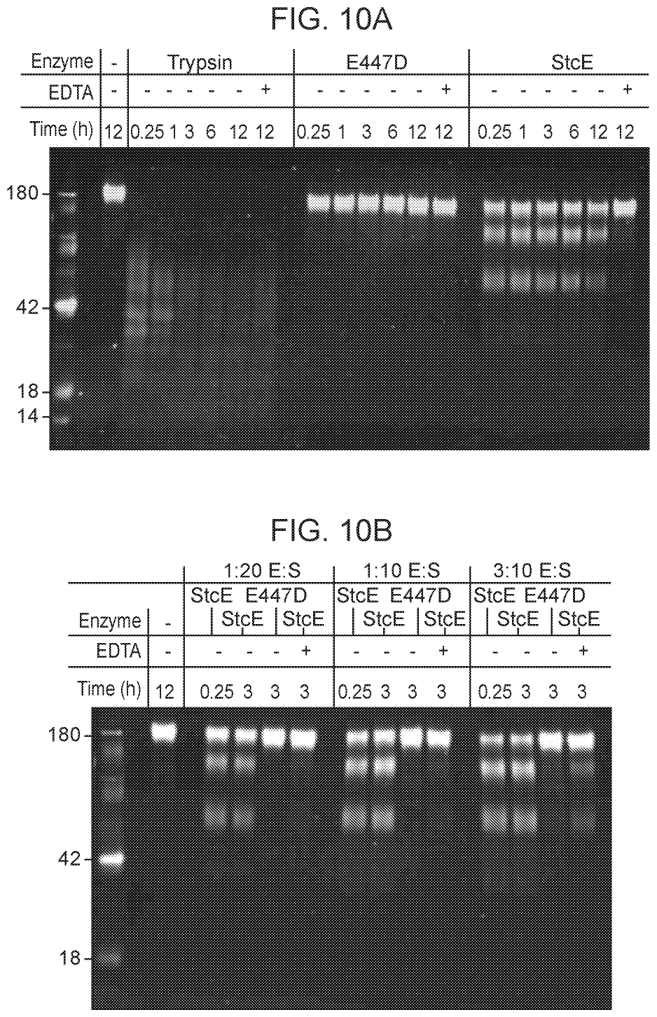

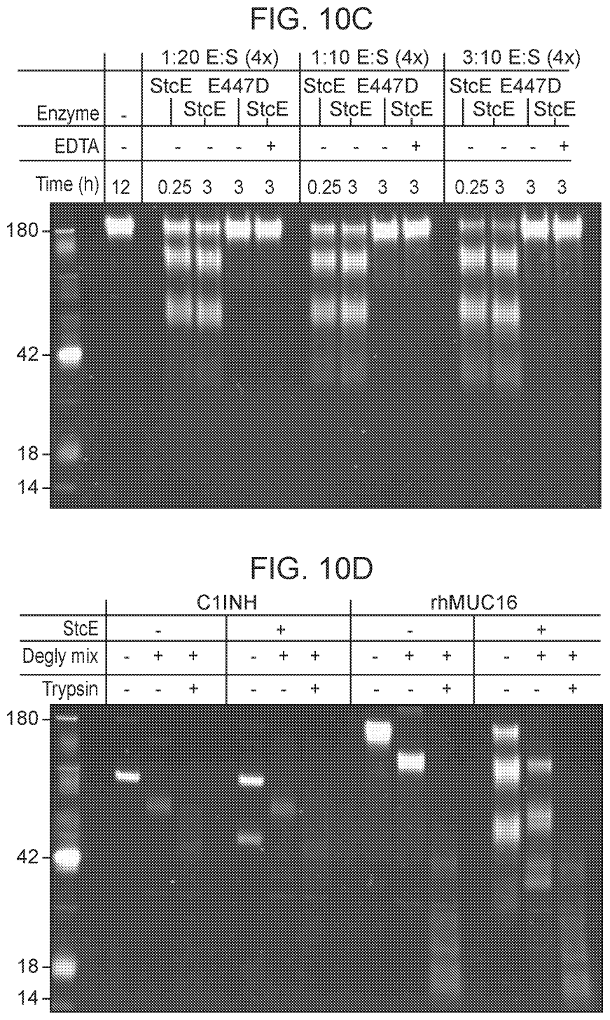

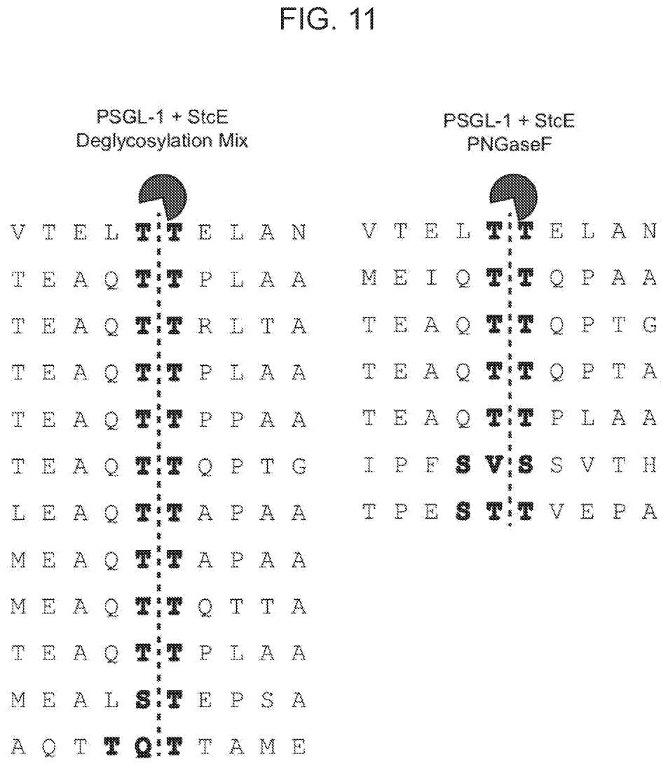

StcE has a distinct peptide consensus sequence, S/T*-X-S/T. In a further step, it was investigated whether StcE had a preferred sequence or structure recognition motif. The recombinant mucin-domain glycoproteins (recombinant MUC16, podocalyxin, CD43, PSGL-1, Syncam-1, and CD45) were digested with StcE, de-N-glycosylated with PNGaseF, trypsinized, and subjected to MS analysis using an optimized protocol (see FIG. 10). Through manual validation of peptides present in the StcE samples but not in the control samples (PNGaseF and trypsin only), it was discovered that StcE had a distinct peptide consensus sequence, S/T*-X-SIT, where cleavage occurred before the second serine or threonine and X was any amino acid or, to a lesser extent, absent (see panel (a) of FIG. 2, and FIG. 11). As seen from FIG. 2, in N-terminal StcE-cleaved peptides, the P2 (*) position was invariably glycosylated (see panel (b) of FIG. 2). This glycosylation ranged from a single 0-GalNAc residue to higher order structures such as a di-sialylated T antigen, indicating that StcE accepted a variety of glycans at the P2 position. StcE cleavage was also permissive to glycosylation at the P1′ position. In all cases, neither the peptide sequence nor the glycan alone was sufficient to predict cleavage.

A single GalNAc residue is the minimum necessary glycoform for StcE cleavage. Based on MS analysis of cleaved peptides (see panel (b) of FIG. 2), the minimum necessary glycoform for StcE cleavage was a single GalNAc residue. To confirm this, a synthetic glycosylated polypeptide comprising GalNAc-α-O-Ser residues mixed with Lys residues in a random sequence was incubated together with StcE. As seen in panel (c) of FIG. 2, StcE cleaved the glycosylated polymer, and this cleavage was reduced when the polymer was deglycosylated. It was also observed that StcE cleaved a synthetic peptide containing a single GalNAc, RPPIT*QSSL (SEQ ID NO:3), into RPPIT*Q (SEQ ID NO:15) (see panel (d) of FIG. 2). These data confirm that 0-GalNAc was the minimum required glycoform on the P2 position serine or threonine residue. GalNAc is the first glycan found on every site of mucin-type 0-glycosylation and SIT-X-SIT is commonly found in their characteristic proline, threonine, and serine-rich repeat domains. Therefore, StcE is a true mucinase, a protease that specifically cleaves mucins, but is promiscuous within that family.

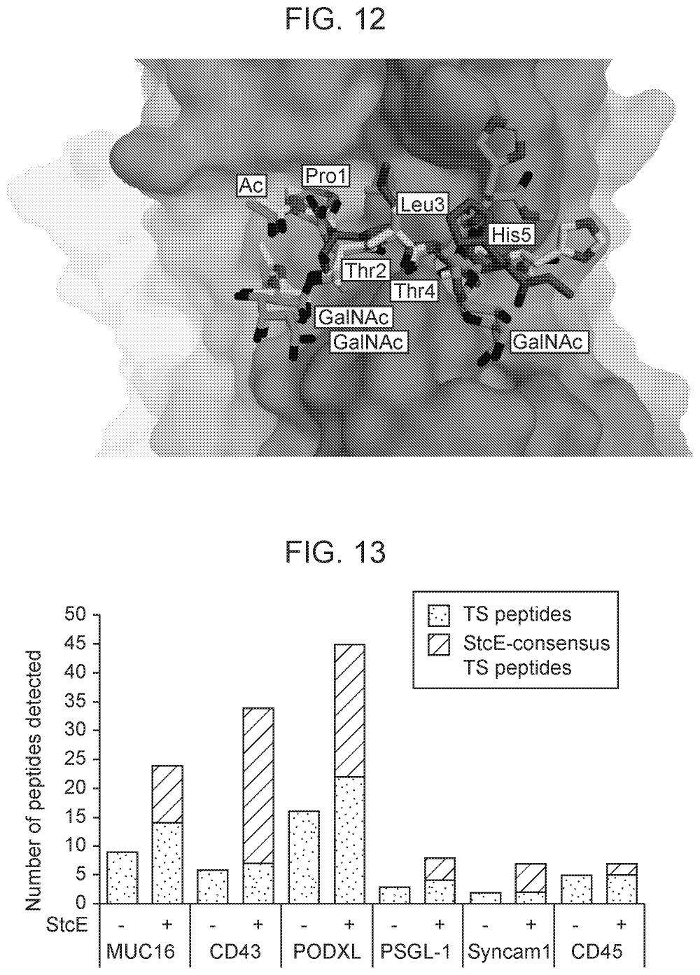

The observed insensitivity of 0-glycosylated but non-mucinous proteins to StcE activity suggested that secondary structure may be an additional recognition determinant. For example, fetuin was not cleaved by StcE (see panel (b) of FIG. 1), although it exhibited a correctly glycosylated StcE consensus sequence GPT*PSAA (SEQ ID NO:16) (*=sialyl T antigen, among others) (see Windwarder et al., J. Proteomics (2014) 108, 258-268). To explore the role of secondary structure in the determination of StcE's specificity, peptide docking studies were conducted with a previously reported crystal structure of StcE (Yu et al., Structure (2012) 20, 707-717) and model glycopeptides derived from a StcE-labile podocalyxin sequence Ac-P(GalNAcα-)TL(GalNAcα-)TH-NMe (SEQ ID NO:4) (see panel (e) of FIG. 2, and FIG. 12). When docked using a scaffold consistent with previously reported zinc metalloprotease/peptide co-crystal structures (see Gomis-Rüth et al., J. Biol. Chem. (2009) 284, 15353-15357), the ligand made specific contacts with the zinc ion and other residues of the catalytic core as well as residues of a flanking β-strand, forming a combined antiparallel β-sheet. The acetyl groups of the GalNAc moieties frequently formed intramolecular contacts with peptide backbone amides. Previous studies have shown that similar carbohydrate-peptide interactions force O-α-GalNAc glycopeptides into a β-strand-like “mucin fold” (see e.g., Coltart et. al., J. Am. Chem. Soc. (2002) 124, 9833-9844). The interactions within the modeled StcE/substrate complex therefore support that StcE may partially achieve its selectivity by recognizing a mucin-fold. Importantly, it appears that in this conformation, the glycan moieties of docked glycopeptides were oriented away from the enzyme's active site (see panel (e) of FIG. 2), which may enable StcE to cleave glycopeptides with larger glycans.

### Example 2: STCE Improves Mass Spectrometry Analysis of Mucin-Domain Glycoproteins

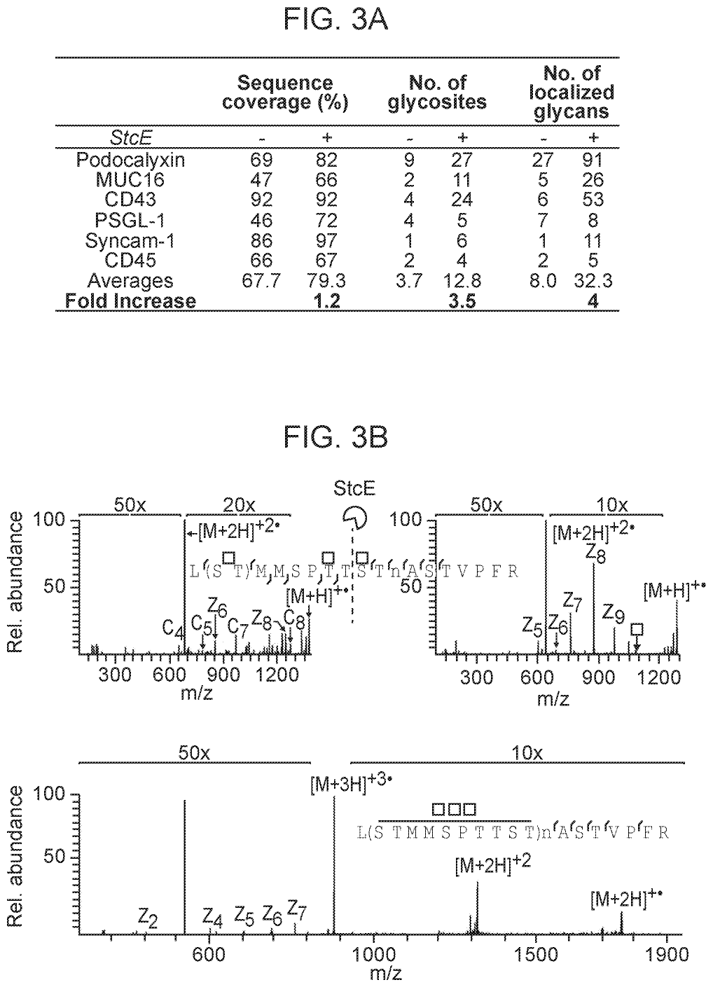

Given its specificity for mucin domains, the incorporation of StcE into common proteomic workflows to facilitate analysis of mucin glycoproteins was investigated. Recombinant substrates (see panel (b) of FIG. 1) were digested with StcE, treated with PNGaseF to remove N-glycans, trypsinized, and subjected to MS. As seen in panel (a) of FIG. 3, StcE treatment increased protein sequence coverage by up to 50%, number of glycosites by up to 6-fold, and number of localized glycans by up to 11-fold, with averages of 20%, 3.5-fold, and 4-fold improvement, respectively. StcE's ability to break up areas of dense O-glycosylation, which generated smaller glycopeptides with higher charge density, contributed to the observed gains. This allowed for better electron transfer dissociation (ETD) spectra, which were necessary for glycosite mapping. To illustrate this concept, ETD spectra of three representative CD43 peptides are shown in panel (b) of FIG. 3. In the untreated sample (bottom panel) site-localization of the three O-GalNAc modifications was not possible, but StcE treatment (top panel) resulted in two peptides covering the same sequence, each with sufficient charge and fragmentation for site-localization of the modification. In silico searches for peptides with serine or threonine at their N-terminus may aid in database searches of StcE-cleaved samples (see e.g., FIG. 13).

### Example 3: STCE Cleaves Native, Human-Derived Mucins in Biological Samples

Cell surface mucins have been implicated as pathogenic drivers of cancerous growth and cancer progression. Tools such as StcE and analogs for their functional analysis provide for detecting abnormal cell growth, cancerous growth and cancer progression.

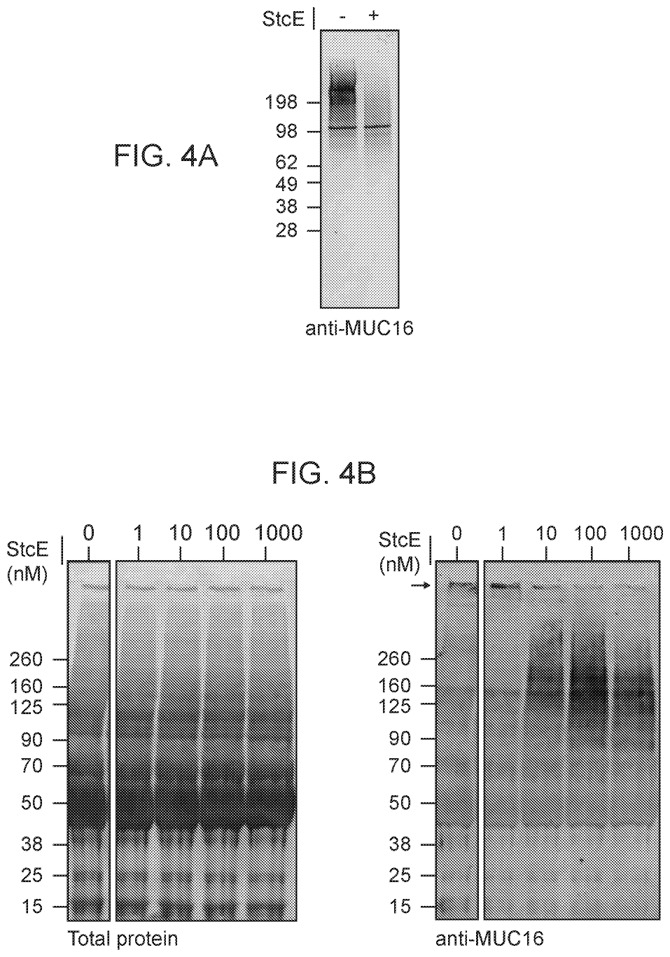

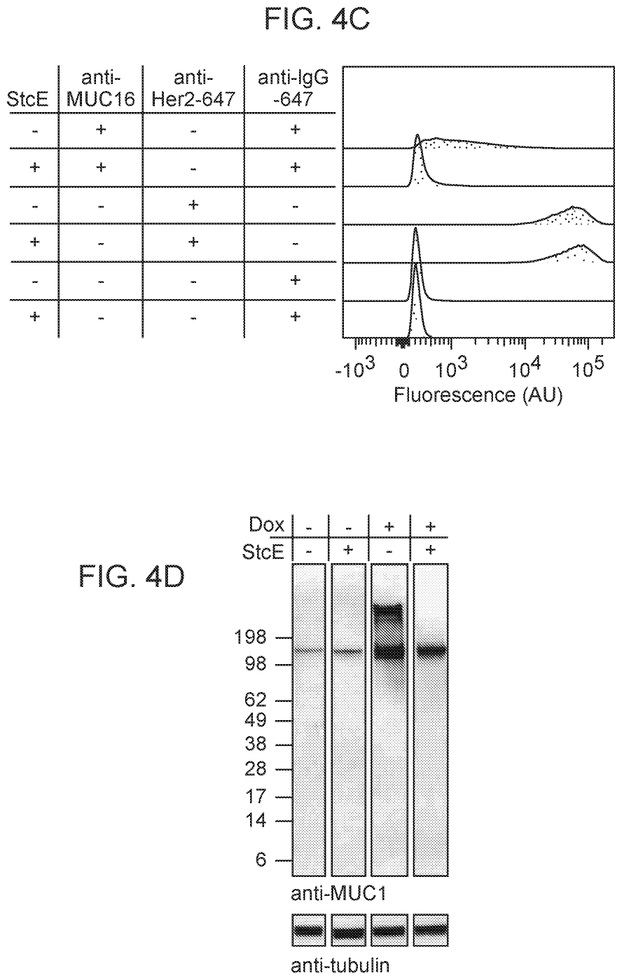

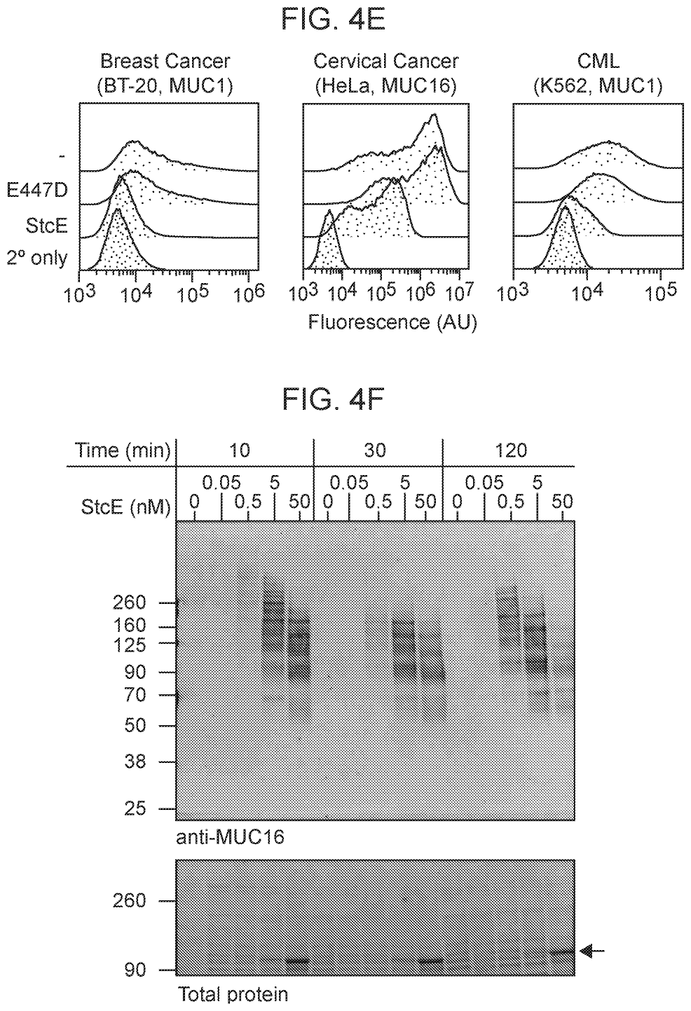

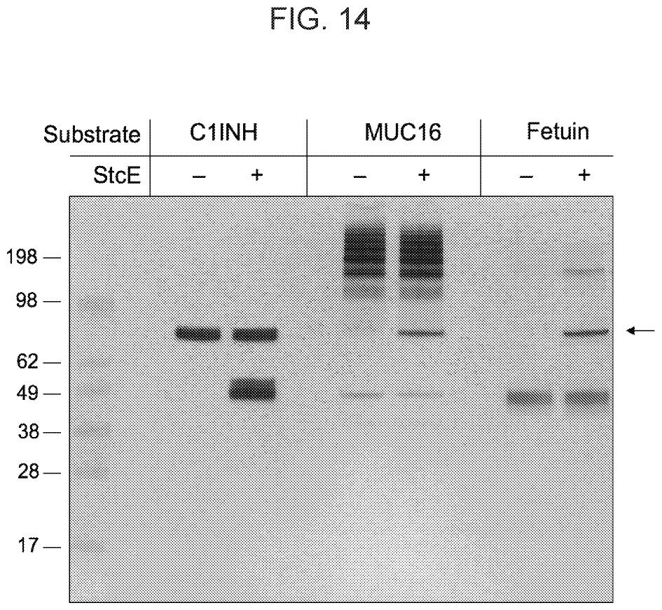

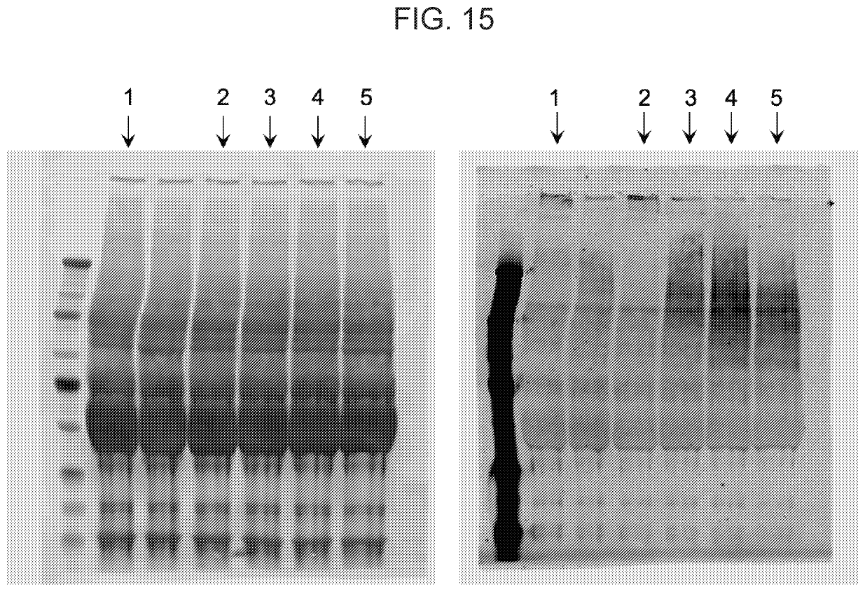

StcE's ability to cleave native, human-derived mucins was confirmed using a commercially available semi-crude preparation of MUC16 from cancer patient ascites fluid. This preparation was found to be sensitive to StcE cleavage, as shown in panel (a) of FIG. 4 and in FIG. 14. The density around 200 kDa in the semi-crude preparation does not originate from full-length glycosylated MUC16, however, which migrates with an apparent molecular weight in the megadalton range. To demonstrate cleavage of full-length human MUC16, crude ascites that had been obtained from an ovarian cancer patient were incubated with StcE. In untreated ascites, density in the stacking gel was detected (see arrow in panel (b) of FIG. 4, and FIG. 15), which was consistent with a very high molecular weight species. StcE treatment for 1 h at 37° C. resulted in a dose-dependent decrease in apparent molecular weight, demonstrating that StcE has activity on human MUC16.

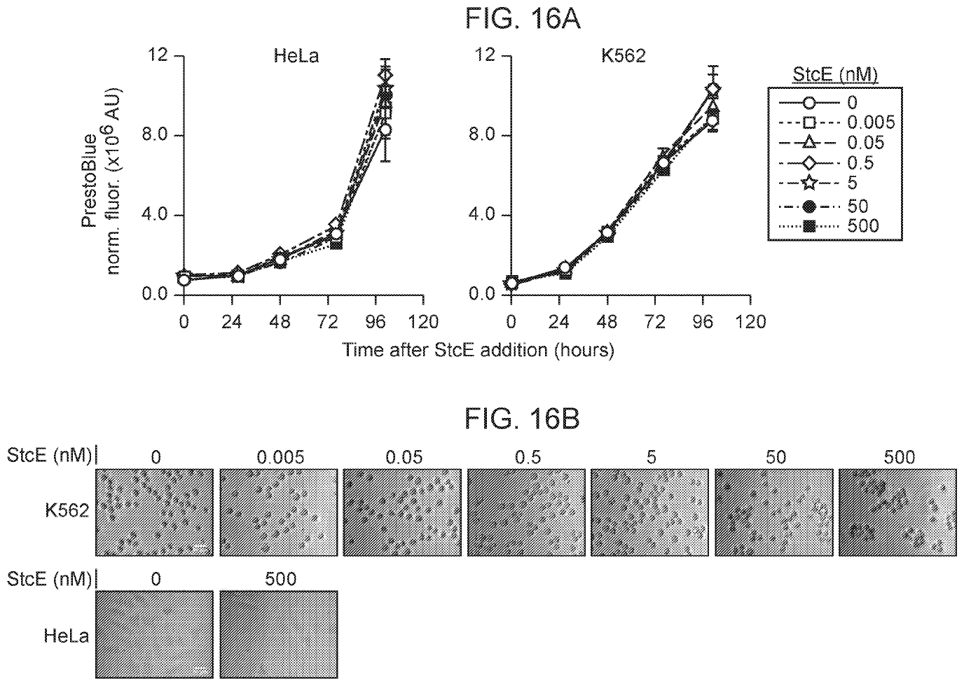

StcE treatment had no negative effect on cell viability, was non-toxic to both adherent and suspension cell lines at all concentrations tested, and did not affect proliferation over days, as shown in FIG. 16.

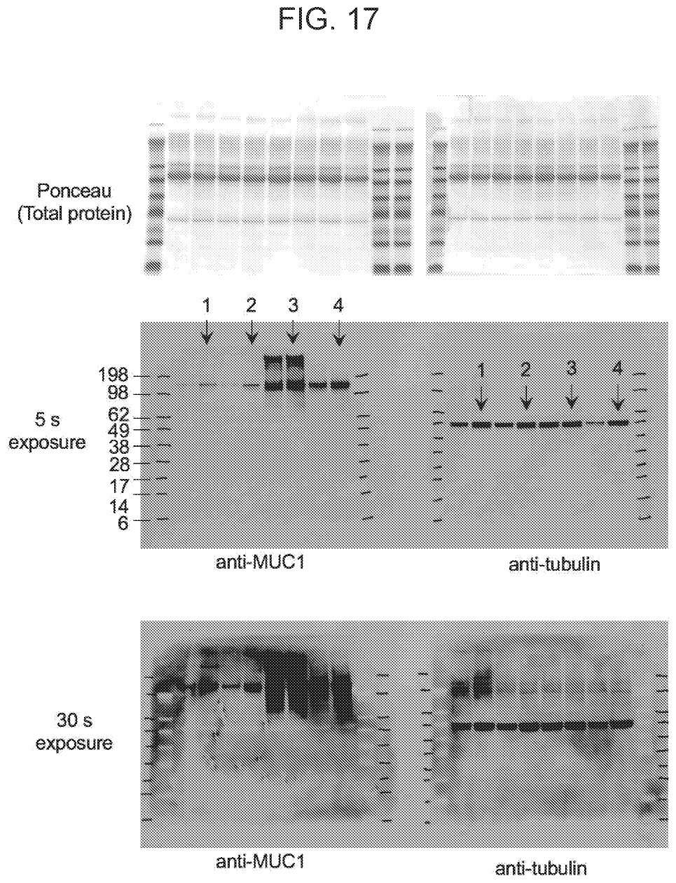

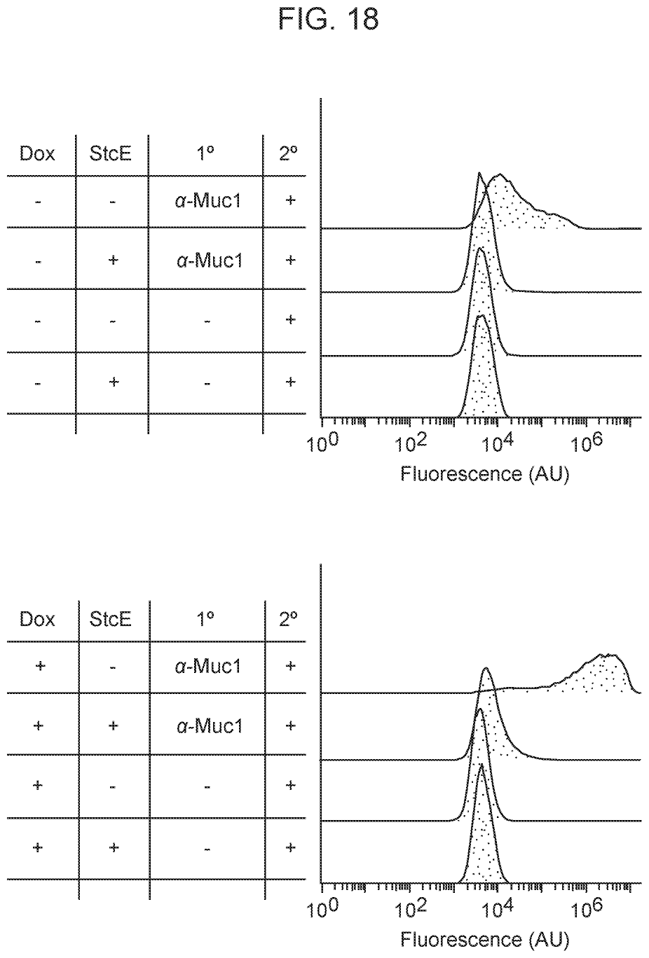

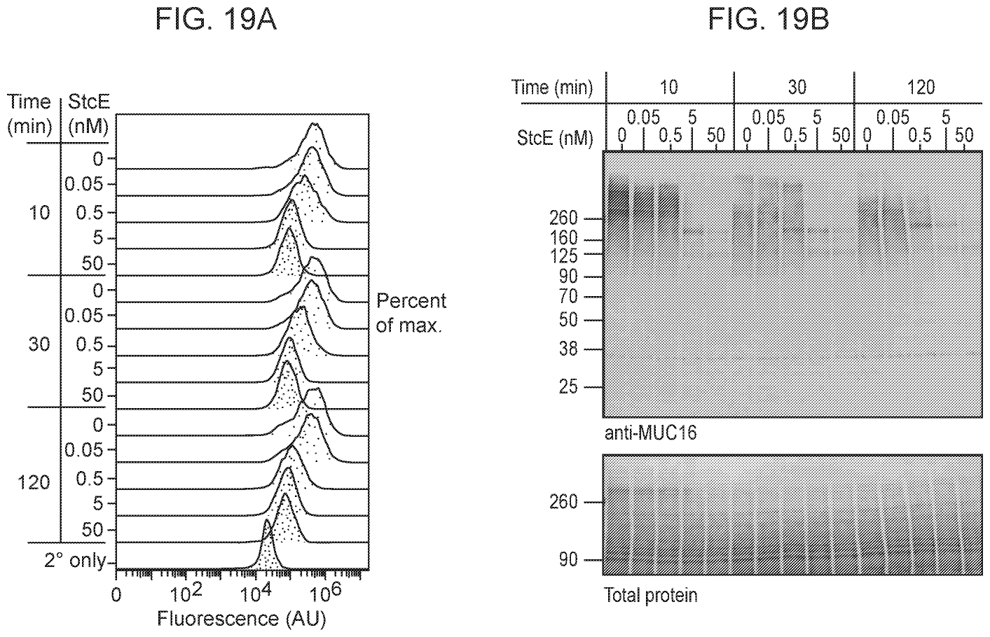

Next, the human breast cancer cell line SKBR3 was treated with StcE and probed for changes in abundance of MUC16. As determined by flow cytometric analysis, StcE depleted MUC16, but had no effect on the highly abundant N-glycosylated but non-mucin HER2 receptor, as seen in panel (c) of FIG. 4. StcE's effect was also tested on breast cancer-associated mucin MUC1 using an MCF10A cell line ectopically expressing a signaling deficient form of this cell-surface mucin (MUC1ΔCT) (see Shurer et al., ACS Biomater. Sci. Eng. 2017). StcE readily cleaved glycosylated MUC1ΔCT but was inactive on an underglycosylated form of MUC1ΔCT (˜125 kD) which is also visible on the Western blot, as shown in panel (d) of FIG. 4 as well as in FIG. 17 and FIG. 18. Further, StcE's cleaving activity did not appear to be cell-line dependent, as it digested cell surface MUC1 and MUC16 from cell lines derived from a variety of cancer types (see panel (e) of FIG. 4). These results confirmed that StcE retained its activity in cellulo. Furthermore, the supernatants of StcE-treated HeLa cells, but not their vehicle only-treated (control) counterparts, stained strongly for MUC16, and the apparent molecular weight of the mucin fragments decreased with increasing StcE concentration and treatment time, as shown in panel (f) of FIG. 4 and in FIG. 19.

These results confirm StcE as an effective and powerful tool to release and solubilize mucins from biological samples useful in detecting abnormal cell growth, cancerous growth and cancer progression.

### Example 4: STCE Purifies Mucins from Protein Mixtures

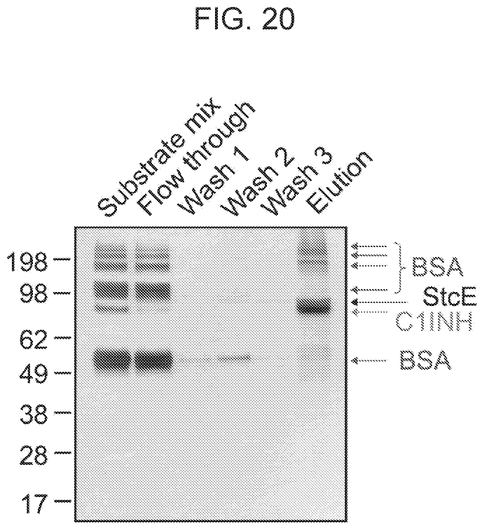

The application of StcE to purify mucins from protein mixtures was also evaluated. For this purpose, StcE was conjugated to beads using reductive amidation. A pulldown using a mixture of BSA and C11NH showed enrichment of C11NH in the elution (see FIG. 20), indicating StcE's utility as an enrichment tool.

### Example 5: STCE Treatment of Cultured Cells Reveals that Siglec-7 Binds Mucin-Domain Glycoproteins

Based on StcE's specificity for mucins, the use of StcE's to discover mucin-based ligands of glycan-binding receptors whose physiological binding partners were unknown was investigated.

Evidence supports that so-called glycan-binding proteins can recognize discrete glycoprotein or glycolipid ligands via motifs that encompass both glycan structures as well as elements of their underlying scaffolds. As a landmark example, PSGL-1 was identified as a cell-surface mucin that functioned as the chief ligand for P-selectin at sites of inflammatory leukocyte recruitment (Pouyani et al., Cell (1995) 83, 333-343); notably, PSGL-1 was effectively digested with StcE, as shown in panel (b) of FIG. 1. The molecular determinants of PSGL-1 that confer P-selectin binding included a specific O-glycan structure combined with a nearby peptide motif (Somers et al., Cell (2000) 103, 467-479). Likewise, the immune modulatory receptor PILRα recognized a composite mucin-derived sialoglycopeptide epitope on cognate ligands (Kuroki et al., Proc. Natl. Acad. Sci. (2014) 111, 8877-8882).

StcE's ability to cleave mucins facilitate the identification of mucins as binding partners of orphan receptors such as the Siglecs was investigated.

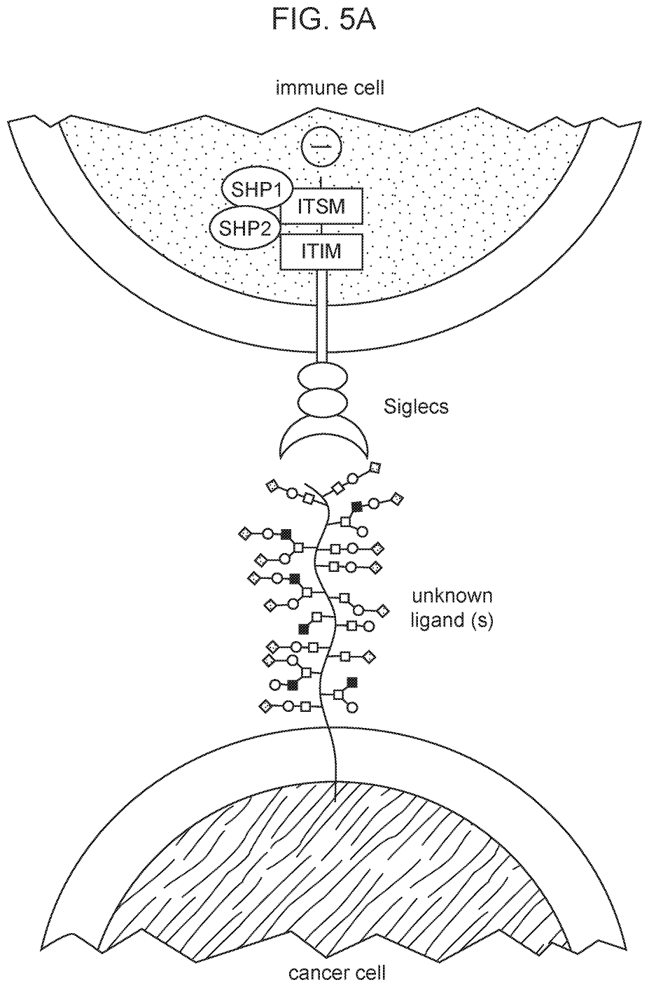

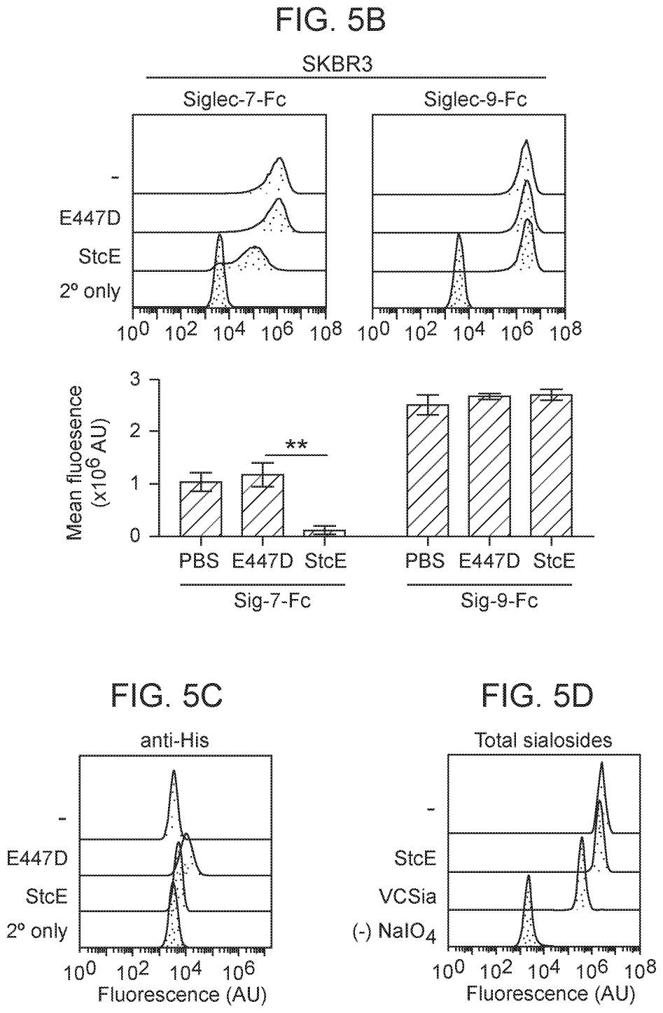

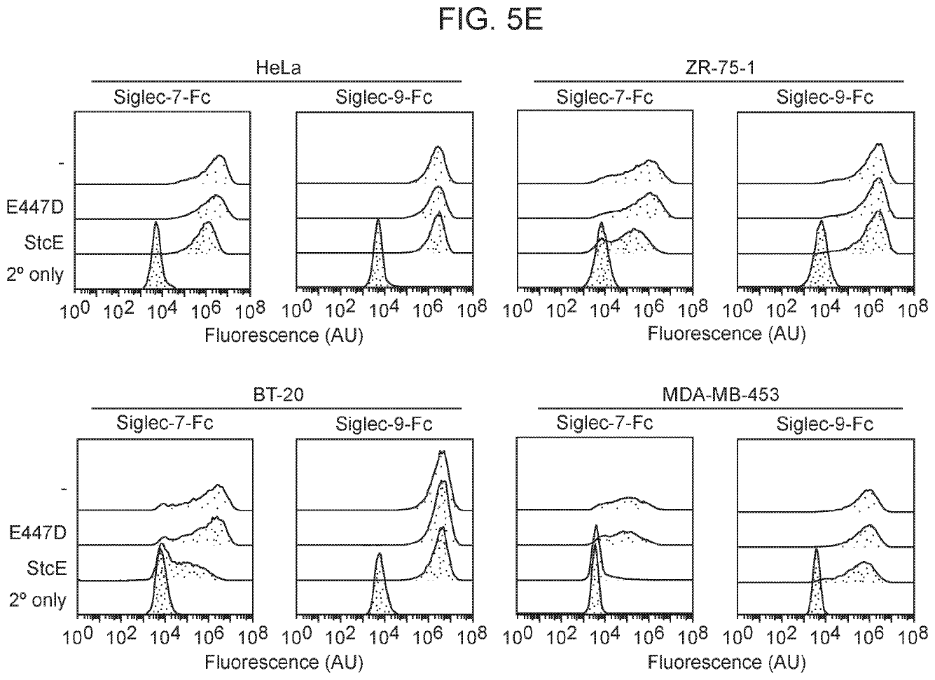

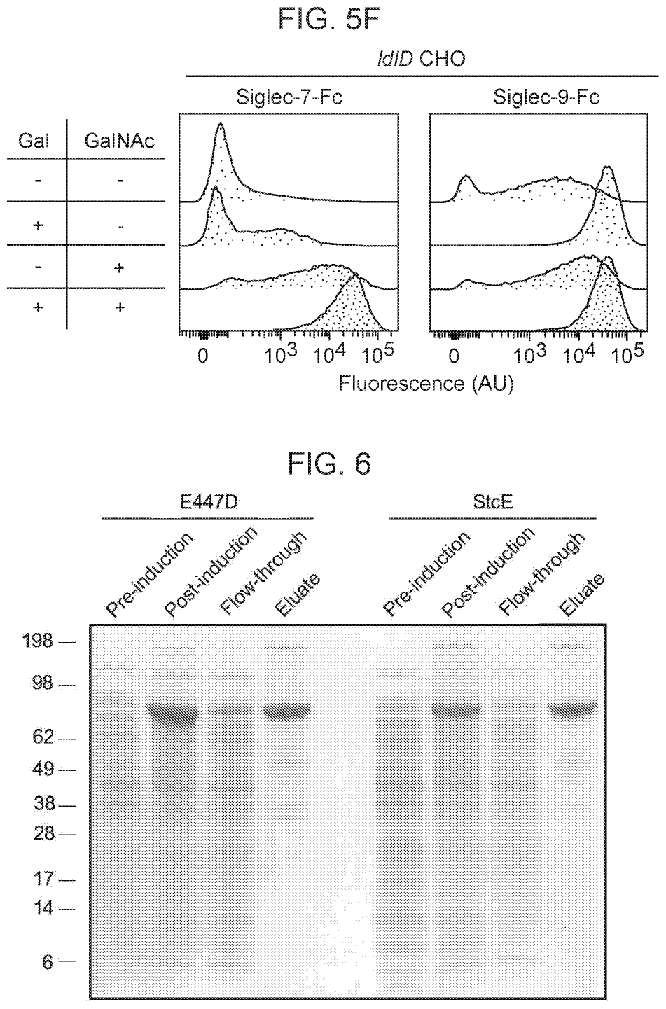

Sialic acid-binding immunoglobulin-type lectins (Siglecs) are a glycan-binding receptor family whose physiological ligands are largely unknown. Individual family members exhibit preferences for sialosides of various linkages to underlying glycan motifs, but the specific glycoproteins or glycolipids they interact with in biological settings are not fully known. Siglecs-7 and -9 have been implicated as inhibitory receptors that function similarly to the immune checkpoints PD-1 and CTLA-4, the targets of several successful cancer immune therapies (Sharma et al., Cell (2015) 161, 205-214). Extracellularly, Siglec-7 and -9 each have a sialic acid-binding Vset domain (see panel (a) of FIG. 5). Intracellularly, they resemble PD-1, with C-terminal cytosolic tyrosine-based inhibitory motif (ITIM) and tyrosine-based switch motif (ITSM) domains that mediate inhibitory signaling. Enzymatic removal of sialic acids en masse from cancer cell surfaces enhances immune cell mediated clearance of those cells through loss of Siglec-7 and -9 binding. Despite years of effort, however, ligands of Siglec-7 and -9 have not been identified.

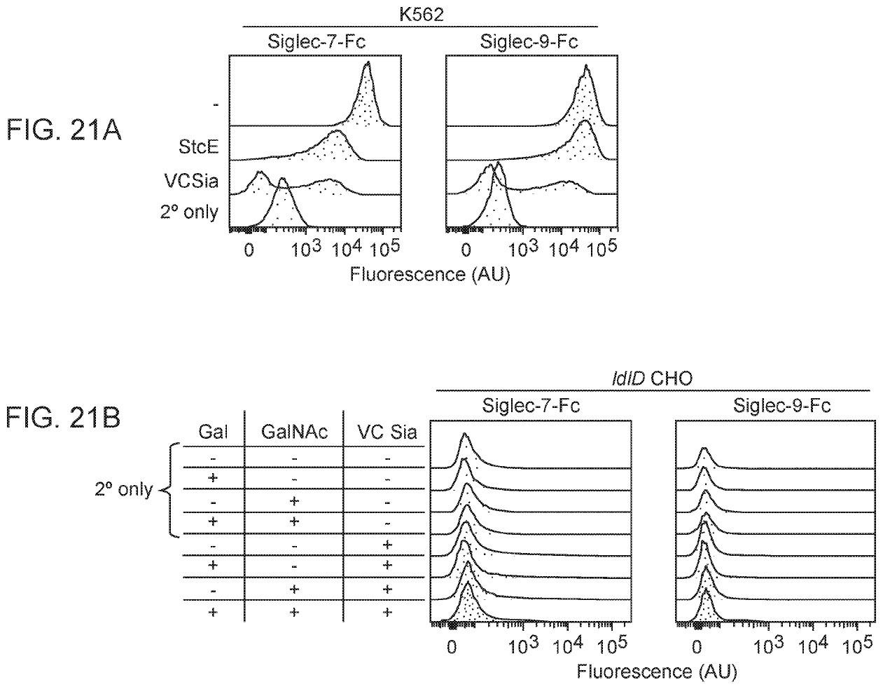

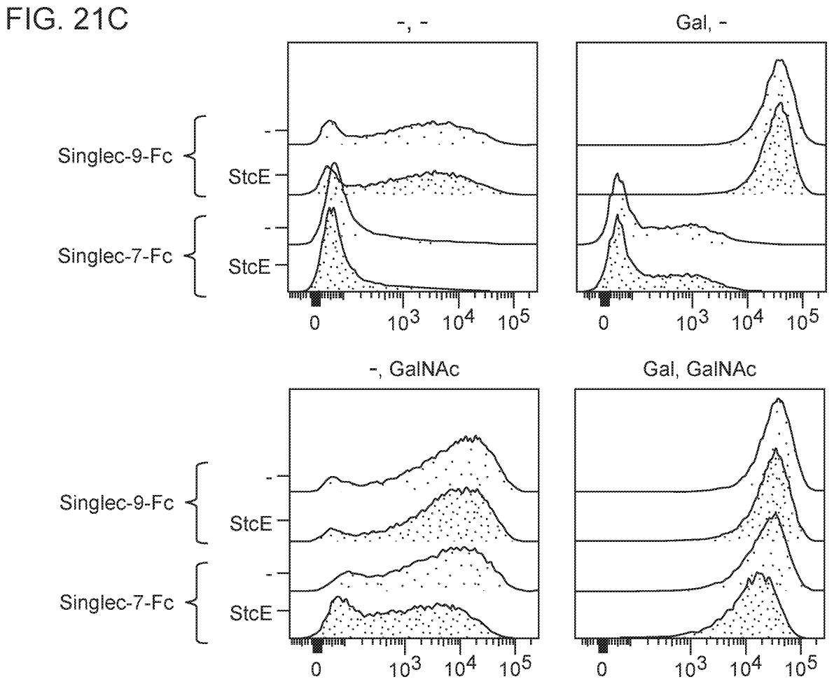

Using soluble Siglec-Fc fusions, the effects of StcE treatment on Siglec-7 and -9 binding to SKBR3 cells were assessed. Analysis by flow cytometry showed that StcE treatment depleted Siglec-7-Fc binding but had no effect on Siglec-9-Fc binding, as shown on the top of panel (b) of FIG. 5 for flow cytometry histograms, and on the bottom of panel (b) in FIG. 5 for biological replicates. The inactive StcE point mutant E447D had no effect on the binding of either Siglec-Fc. Siglec-7- and -9-Fc binding was confirmed to be dependent on sialic acid via treatment with Vibrio cholerae sialidase (see FIG. 21). These results suggested that Siglec-7 recognized mucin glycoproteins on SKBR3 cells but that Siglec-9 bound structures that were resistant to StcE treatment.

In order to ensure that StcE did not simply bind to cell surface mucins and block accessibility to Siglec-7-Fc, SKBR3 cells that were treated with either StcE or E447D were stained with anti-His antibodies to bind the His-tagged enzymes (see panel (c) of FIG. 5). E447D bound cell surfaces more tightly than StcE did, but did not deplete Siglec-7-Fc binding, indicating that StcE's enzymatic activity was required for its effects on Siglec-7-Fc. Interestingly, periodate-mediated labeling of cell surface sialic acids revealed that StcE treatment had only a minor effect on total cell-surface sialic acid levels (see panel (d) of FIG. 5). Thus, StcE removed only a small fraction of total sialosides while depleting a majority of Siglec-7-Fc binding structures.

A panel of cell lines was tested for StcE-mediated depletion of Siglec-7 and -9 ligands. In all other cell lines tested, Siglec-7-Fc binding decreased upon StcE digestion while Siglec-9-Fc binding remained unchanged, as shown in panel (e) of FIG. 5 and in FIG. 21).

To further support the identification of Siglec-7 as a sialomucin-binding receptor, ldlD Chinese Hamster Ovary (CHO) cells were employed, which were deficient in UDP-glucose/galactose-4-epimerase (GALE). GALE interconverts UDP-glucose and UDP-GlcNAc to UDP-galactose and UDP-GalNAc, respectively. Without active GALE, ldlD CHO cells can still take up glucose from tissue culture media and use it to biosynthesize nucleotide sugars of glucose, mannose, fucose, and sialic acid. However, they cannot initiate or elaborate their glycans with GalNAc or galactose, resulting in truncated cellular glycans. Supplementing the media with 10 μM galactose and 100 μM GalNAc rescues the phenotype, as these undergo conversion to the respective nucleotide sugars within cells.

Unrescued ldlD CHO cells exhibited weak binding by both Siglec-7- and -9-Fc (see panel (f) of FIG. 5). Siglec-9-Fc binding increased by approximately the same amount after rescue with galactose alone and with both galactose and GalNAc supplementation, but increased only slightly with GalNAc rescue alone (see panel (f) of FIG. 5, right). These results were consistent with a view that Siglec-9 ligands are predominantly non-mucinous, as GalNAc deficiency should abrogate mucin-type O-glycosylation. Siglec-7-Fc binding was largely unaffected by galactose supplementation alone, but increased with GalNAc supplementation. Rescue with both sugars increased Siglec-7-Fc binding further (see panel (f) of FIG. 5, left). In all conditions, both Siglec-7- and -9-Fc binding were sialidase sensitive, confirming their dependence on sialic acid (see FIG. 21). In addition, StcE treatment had no effect on Siglec-9-Fc binding across any rescue condition, but decreased Siglec-7-Fc binding in cases with GalNAc supplementation (see FIG. 21).

These results distinguish the specificities of Siglec-7 and Siglec-9 on cell surfaces. In the case of Siglec-7, it appears that glycoprotein ligands may exist, and that at least a subset of such ligands are mucin-domain glycoproteins which may provide an avenue for immune checkpoint interventions.

The role mucin-domain glycoproteins play in immunological signaling is not limited to Siglec-7. For example, receptors such as CD45 and TIM-3, which are emerging as critical players in healthy immune function and the immune response to cancer, contain prominent mucin domains that are considered necessary for their activities. Further, several members of the galectin family, which are pro-oncogenic glycan-binding proteins, are known mucin-binders, but their specificities for discrete glycoproteins have not been fully characterized. Enzymatic de-mucination with StcE provides a powerful tool for de-orphanizing the receptors and ligands that interact with mucin-domain glycoproteins.

### Example 6: Identification and Characterization of Mucinases for Mucin Staining and Enrichment

### Methods

### Determination of Mucinase Consensus Motif

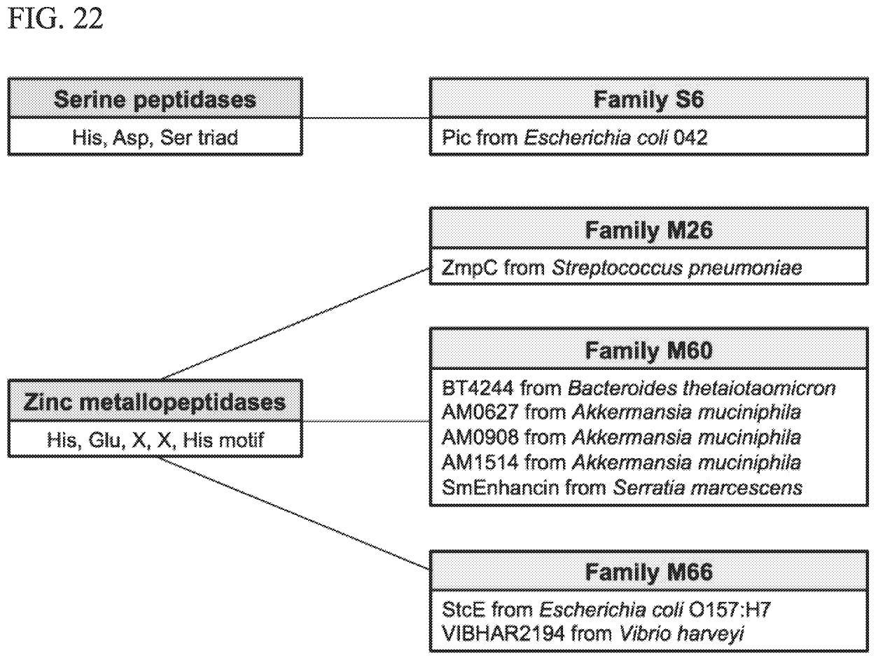

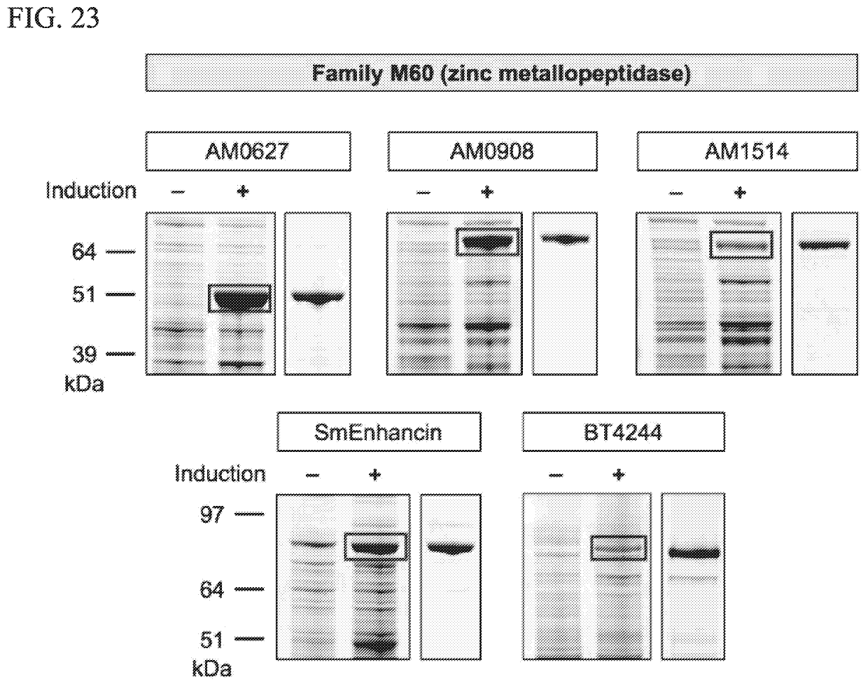

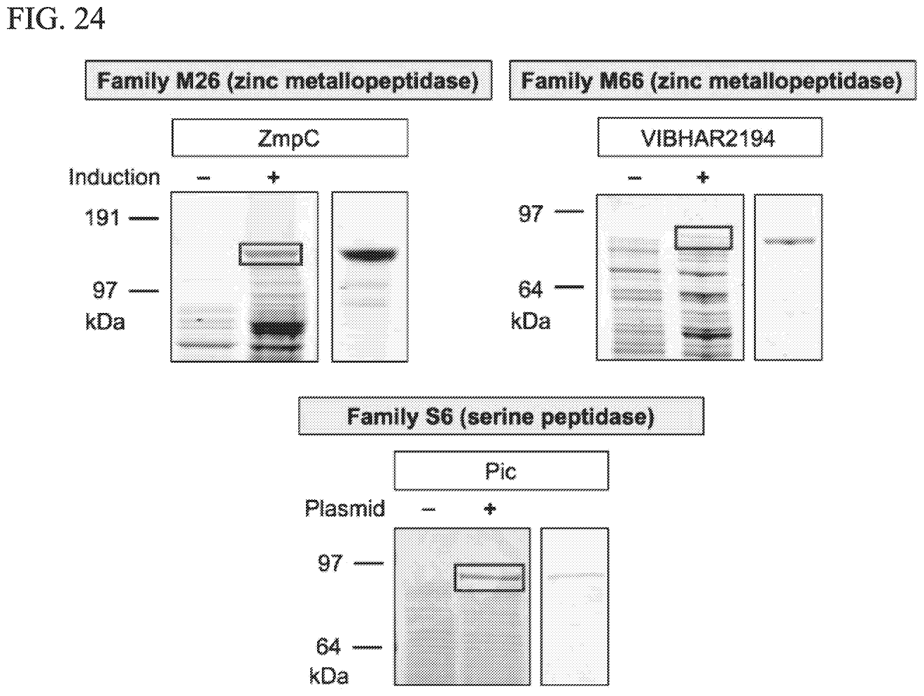

Candidate mucinases were identified and grouped into peptidase families (FIG. 22). Candidate mucinases were expressed and purified for analysis (FIGS. 23-24).

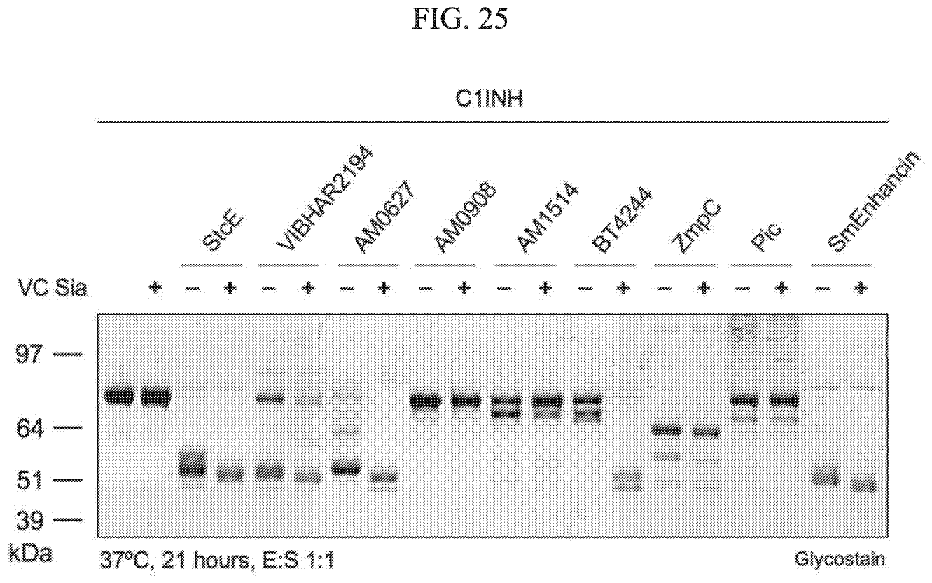

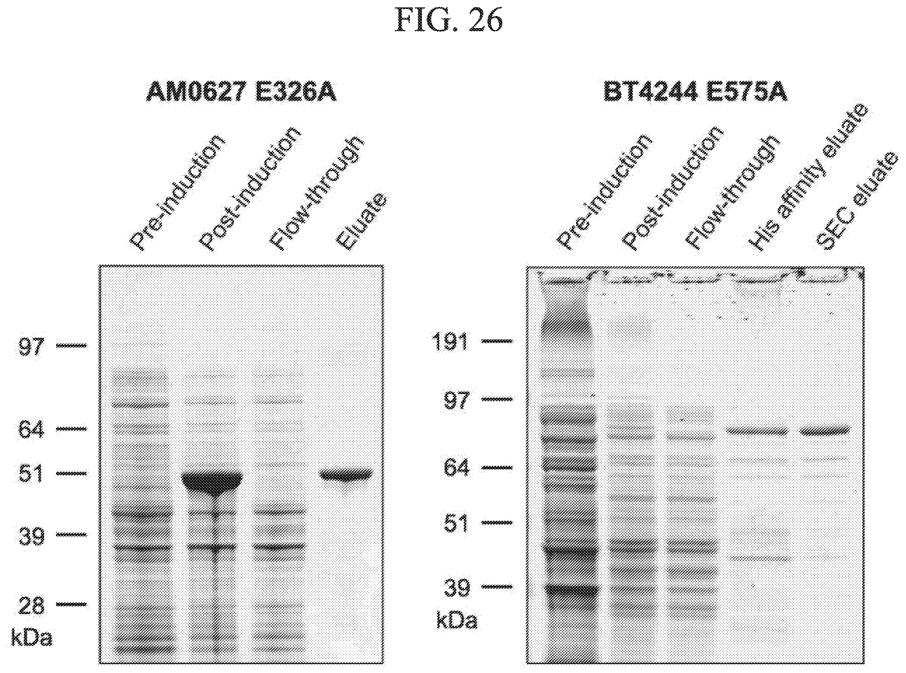

Candidate mucinases exhibit unique activities against native mucins (FIG. 25). Recombinant mucinases were incubated at a 1:1 enzyme:substrate (E:S) ratio with 0.5 μM human plasma-derived C1 esterase inhibitor (C11NH) for 21 h at 37° C. either with or without 10 nM Vibrio cholerae sialidase (VC Sia). Digests were separated by SDS-PAGE and glycosylated peptides were visualized with Pro-Q Emerald 300 Glycoprotein Stain, exhibiting differences in mucinase-generated products and mucinase sensitivity to sialic acid.

Sample Preparation. Four recombinant glycoproteins (CD43, rhMUC16, podocalyxin, and PSGL-1) were digested with the individual mucinases in a 1:1 E:S ratio, in a total volume of 12-13 μL of buffer (50 mM ammonium bicarbonate, pH 7.5) overnight at 37° C. Control proteins were incubated at 37° C. overnight in a solution containing buffer only. Afterward, the volume was increased to 19 μL with buffer. PNGaseF (1 μL; Promega) was added to 99 μL of 50 mM ammonium bicarbonate, and 1 μL of this reaction was added to each reaction vial. Deglycosylation reactions were incubated overnight (12-16 h) at 37° C. Reduction and alkylation were performed according to ProteaseMax (Promega) protocols. Briefly, the solution was diluted to 93.5 μL with 50 mM ammonium bicarbonate. Then, 1 μL of 0.5 MDTT was added and the samples were incubated at 56° C. for 20 min., followed by the addition of 2.7 μL of 0.55 M iodoacetamide at room temperature for 15 min. in the dark. Digestion was completed by adding sequencing-grade trypsin (Promega) in a 1:20 enzyme:protein ratio for 8 h at 37° C. and quenched by adding 0.3 μL of glacial acetic acid. Samples were brought to 1 mL in 0.1% formic acid in water (solvent A) and subjected to C18 clean up using Strata-X columns (Phenomenex). Briefly, the column was washed with 1 mL of solvent B (80% acetonitrile with 0.1% formic acid), followed by equilibration with 1 mL solvent A. The sample (1 mL) was loaded onto the column and washed with 1 mL of solvent A. Finally, peptides were eluted with 300 μL of solvent B and taken to dryness.

Mass spectrometry. Samples were reconstituted in 10 μL of solvent A and analyzed by online nanoflow LC-MS/MS using an Orbitrap Fusion Tribrid mass spectrometer (Thermo Fisher) coupled to a Dionex Ultimate 3000 HPLC (Thermo Fisher). A portion of the sample (4 μL of 10; 40%) was loaded via autosampler onto a C18 nano pre-column using 0.1% formic acid in water (“Solvent A”). For pre-concentration and desalting, the column was washed with 2% ACN and 0.1% formic acid in water (“loading pump solvent”). Subsequently, the C18 nano pre-column was switched in line with the C18 nano separation column (75 μm×250 mm EASYSpray (Thermo Fisher) containing 2 μm C18 beads) for gradient elution. The column was held at 40° C. using a column heater in the EASY-Spray ionization source (Thermo Fisher). The samples were eluted at a constant flow rate of 0.3 μL/min using a 90-minute gradient and a 140-minute instrument method. The gradient profile was as follows (min:% solvent B, 2% formic acid in acetonitrile) 0:3, 3:3, 93:35, 103:42, 104:95,109:95, 110:3, 140:3. The instrument method used an MS1 resolution of 60,000 at FWHM400 m/z, an AGC target of 3e5, and a mass range from 300 to 1,500 m/z. Dynamic exclusion was enabled with a repeat count of 3, repeat duration of 10 s, exclusion duration of 10 s. Only charge states 2-6 were selected for fragmentation. MS2s were generated at top speed for 3 s. HCD was performed on all selected precursor masses with the following parameters: isolation window of 2 m/z, 28-30% collision energy, orbitrap (resolution of 30,000) detection, and an AGC target of 1e4 ions. ETD was performed if (a) the precursor mass was between 300-1000 m/z and (b)3 of 7glyco fingerprint ions (126.055, 138.055, 144.07, 168.065, 186.076, 204.086, 274.092, 292.103) were present at +/−0.1 m/z and greater than 5% relative intensity. ETD parameters were as follows: calibrated charge-dependent ETD times, 2e5 reagent target, precursor AGC target 1e4.

Mass spectrometry data analysis. Raw files were searched using Byonic by ProteinMetrics against the Uniprot human proteome (downloaded Jun. 26, 2016) and/or directed databases containing the recombinant protein of interest. Search parameters included semi-specific cleavage specificity at the C-terminal site of R and K. Mass tolerance was set at 10 ppm for MS1s, 0.35 for MS2s. Methionine oxidation (common 2), asparagine deamidation (common 2), and N-term acetylation (rare 1) were set as variable modifications with a total common max of 3, rare max of 1. O-glycans were also set as variable modifications (common 2), using the “O-glycan 6 most common” database. Cysteine carbaminomethylation was set as a fixed modification. Peptide hits were filtered using a 1% FDR.

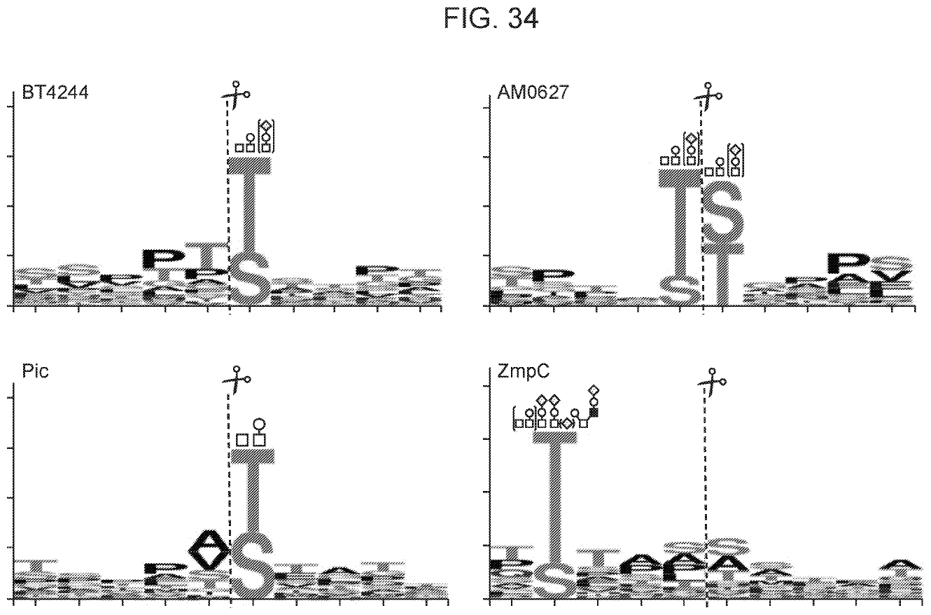

FIG. 34 shows mucinase consensus motifs. Cleaved peptides present in the mucinase-digested samples, but not in the trypsin-only samples, were loaded into WebLogo (weblogo.berkeley.edu). Glycan assignments were assessed manually. Brackets indicate glycans with only a few examples of cleavage, parentheses indicate that the linkage for the second sialic acid of the disialylated structure could not be assigned.

### Staining of Mucin-Domain Glycoproteins Using Inactive Point Mutant Mucinases

FIG. 26 illustrates the recombinant expression and purification of the inactive point mutants AM0627 E326A and BT4244 E575A. AM0627 E326A and BT4244 E575A were purified via His affinity chromatography, with an additional size exclusion chromatography (SEC) step for BT4244 E575A. Protein bands were detected with Coomassie stain (Bulldog-Bio).

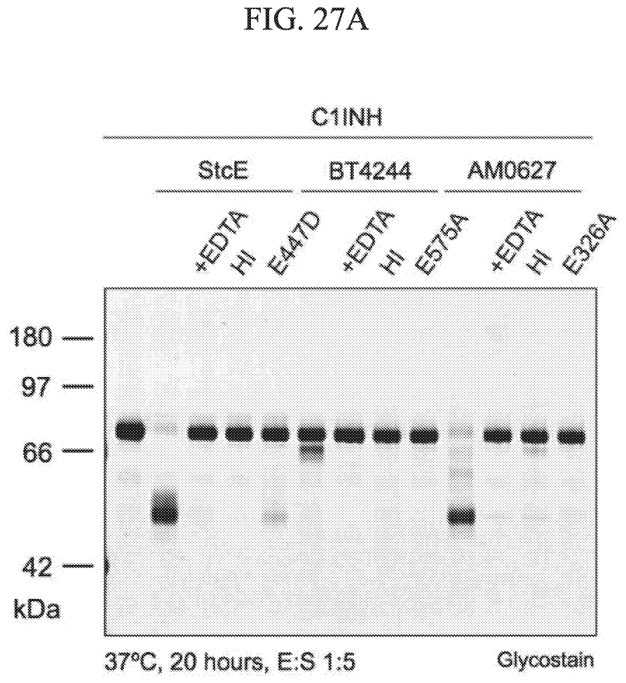

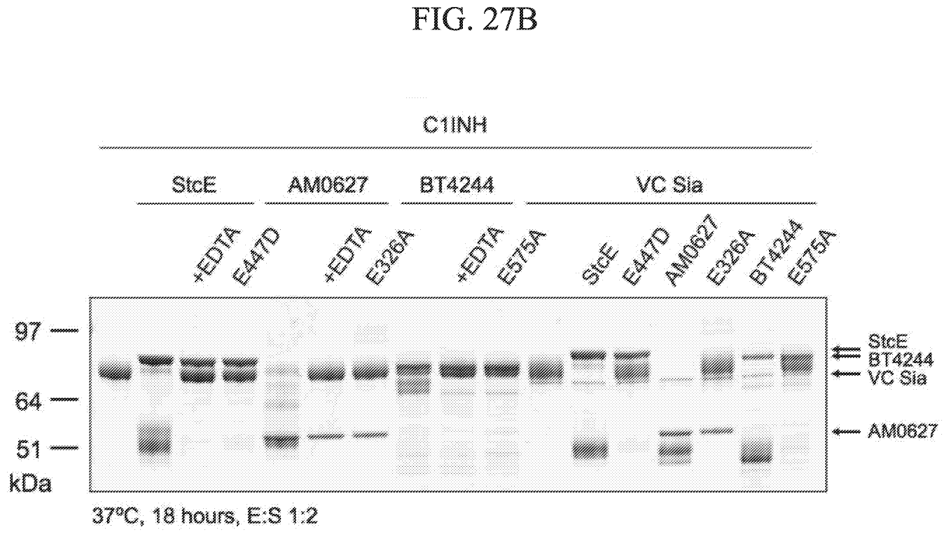

FIGS. 27A-27B illustrate the decrease in catalytic activity for StcE E447D, BT4244 E575A and AM0627 E326A compared to their active enzyme counterparts. In FIG. 27A, 1 μM C11NH was treated with the appropriate mucinases at an E:S ratio of 1:5 for 20 h at 37° C. The activities of the point mutants were compared to other forms of enzyme inactivation, including addition of 25 mM EDTA and heat inactivation (HI) at 65° C. for 10 minutes. Glycosylated fragments were visualized with Pro-Q Emerald 300 Glycoprotein Stain. In FIG. 27B, mucinase activity was tested at high concentration (1 μM) and higher E:S ratio against C11NH at 37° C. for 18 h with or without the addition of 10 nM VC Sia. Proteins were visualized with Coomassie stain (Bulldog-Bio). Little to no mucinase activity was observed in both cases, facilitating binding to mucin substrates without cleavage.

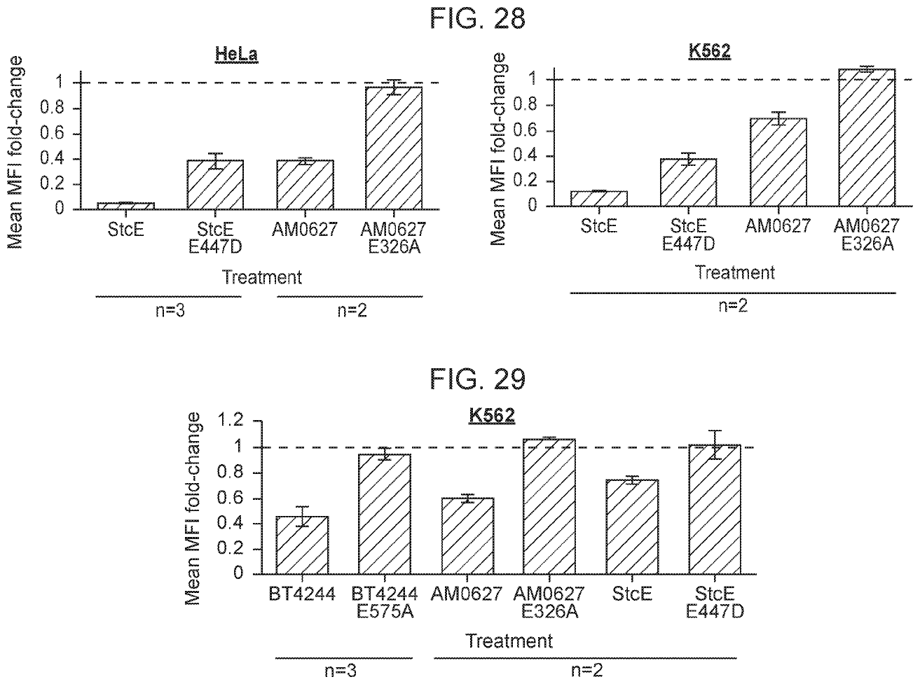

FIG. 28 shows that Alexa Fluor 647-labeled StcE E447D (AF647-E447D) is capable of staining live cells. HeLa cells were treated with 50 nM mucinase for 2 h at 37° C., stained with 50 nM-100 nM (5 μg/mL-10 μg/mL) AF647-E447D for 30 minutes at 4° C., and subjected to live cell flow cytometry. K562 cells were treated with 50 nM mucinase for 2 h at 37° C., stained with 100 nM (10 μg/mL) AF647-E447D for 30 min at 4° C., and subjected to live cell flow cytometry. Fold-change in mean fluorescence intensity with respect to an untreated control (dotted line) is shown. Staining levels were sensitive to pretreatments including removal of mucins with active mucinases and blocking of sites with StcE E447D prior to staining.

FIG. 29 demonstrates that Alexa Fluor 647-labeled BT4244 E575A (AF647-E575A) is capable of staining live cells. K562 cells were treated with 50 nM mucinase for 2 h at 37° C., stained with 100 nM (10 μg/mL) AF647-E575A for 30 minutes at 4° C., and subjected to live cell flow cytometry. Fold-change in mean fluorescence intensity with respect to an untreated control (dotted line) is shown. E575A staining was the most sensitive to pretreatment with its active counterpart compared to pretreatment with other mucinases, reflecting its more selective binding properties.

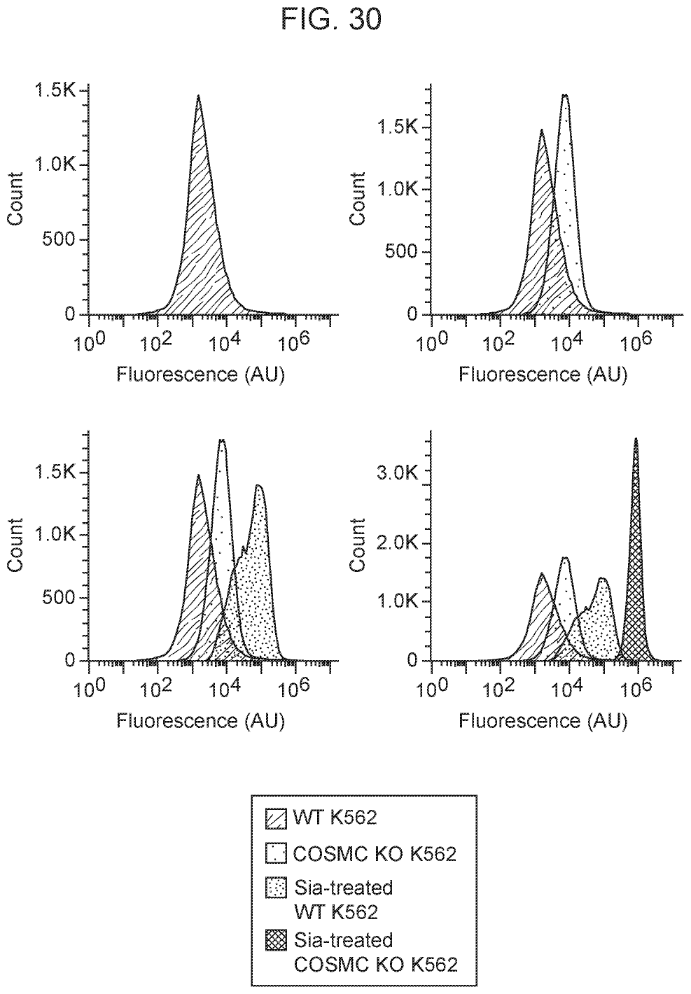

FIG. 30 shows that live cell staining with Alexa Fluor 647-labeled BT4244 E575A (AF647-E575A) increases with knockout of the COSMC chaperone and VC sialidase treatment. Wild-type K562 cells and COSMC knockout K562 cells were incubated with 10 nM VC sialidase for 2 h at 37° C., stained with 100 nM (10 μg/mL) AF647-E575A for 1 h at 4° C., and subjected to live cell flow cytometry. The increase in staining with VC sialidase treatment reflects the sensitivity of BT4244 E575A to terminal sialic acid residues. The highest staining was observed for sialidase-treated COSMC knockout cells, indicating the selectivity of BT4244 E575A for the Tn antigen.

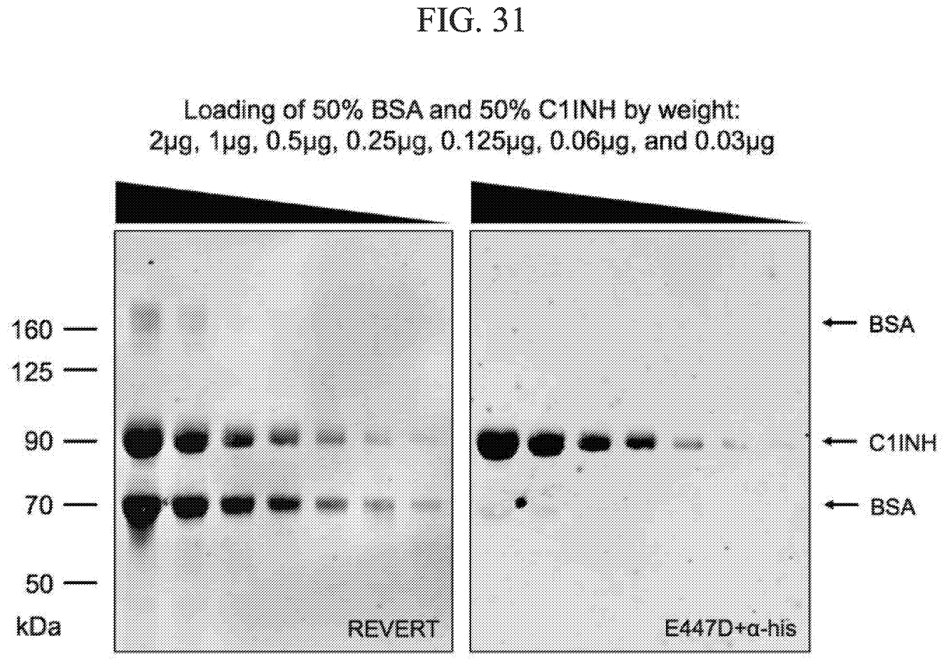

FIG. 31 illustrates that StcE E447D is capable of selectively staining mucin-domain glycoproteins by Western blot. A serially diluted 1:1 mixture of C11NH and bovine serum albumin (BSA) was transferred to a 0.2 μm nitrocellulose membrane and incubated with 20 μg/mL StcE E447D overnight at 4° C. IRdye800CW-labeled ReadyTag anti-6-His (BioX Cell) was used as a secondary. Total protein was visualized using REVERT stain (LI-COR Biosciences). The signal was selective for C11NH over the non-mucin BSA down to 0.03 μg C11NH.

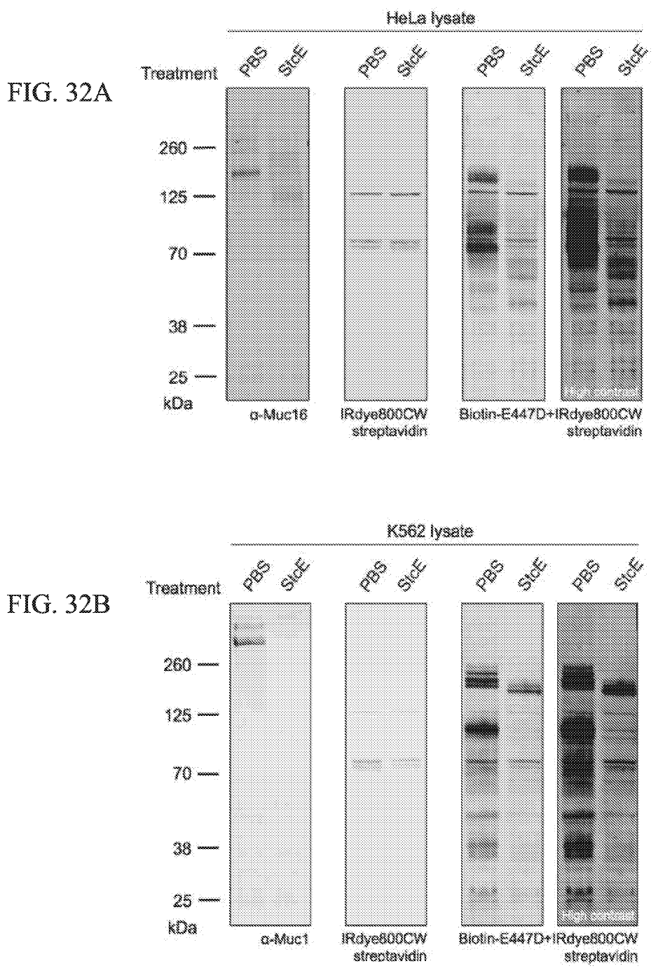

FIGS. 32A-32B show that StcE E447D is capable of identifying StcE-sensitive proteins in cell lysates by Western blot. In FIG. 32A, untreated and StcE-treated HeLa lysates were transferred to a 0.2 μm nitrocellulose membrane and incubated with anti-MUC16 antibody (Abcam, X75) or 10 μg/mL biotin-StcE E447D (1.89 mol biotin/mol E447D). In FIG. 32B, untreated and StcE-treated K562 lysates were transferred to a nitrocellulose membrane and incubated with anti-MUC1antibody (EMD Millipore, 214D4) or 10 μg/mL biotin-StcE E447D. IRdye800CW-streptavidin (LI-COR Biosciences) was used as a secondary for E447D blots and for secondary-only control blots. IRdye800CW goat anti-mouse IgG (LI-COR Biosciences) was used as a secondary for MUC16 and MUC1 blots. In both cell lines, bands corresponding to MUC16/MUC1 and additional StcE-sensitive proteins were visible by E447D staining.

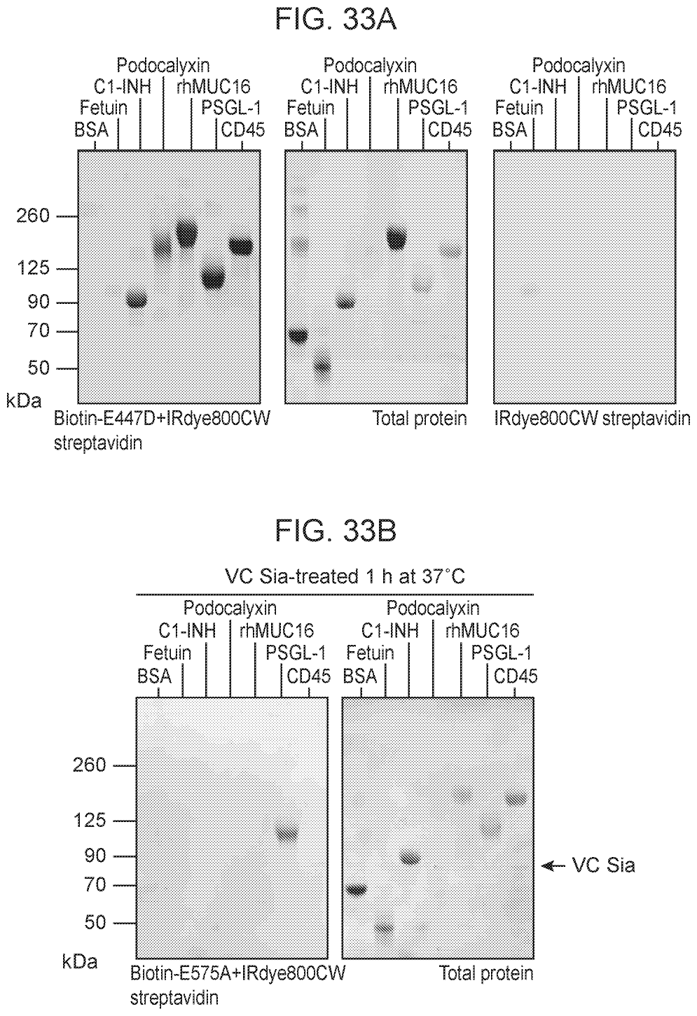

FIGS. 33A-33B demonstrates that StcE E447D is capable of selectively staining a panel of mucin-domain glycoproteins by Western blot while BT4244 E575A stains a subset of this panel. In FIG. 33A, 1 μg of each substrate was transferred to a 0.2 μm nitrocellulose membrane and incubated with 5 μg/mL biotin-StcE E447D (1.89 mol biotin/mol E447D). In FIG. 33B, 1 μg of each substrate was treated with VC sialidase for 1 h a t37° C., transferred to a 0.2 μm nitrocellulose membrane, and incubated with 5 μg/mL biotin-BT4244 E575A (1.37 mol biotin/mol E575A). IRdye800CW-streptavidin (LI-COR Biosciences) was used as a secondary. Total protein was visualized using REVERT stain (LI-COR Biosciences).

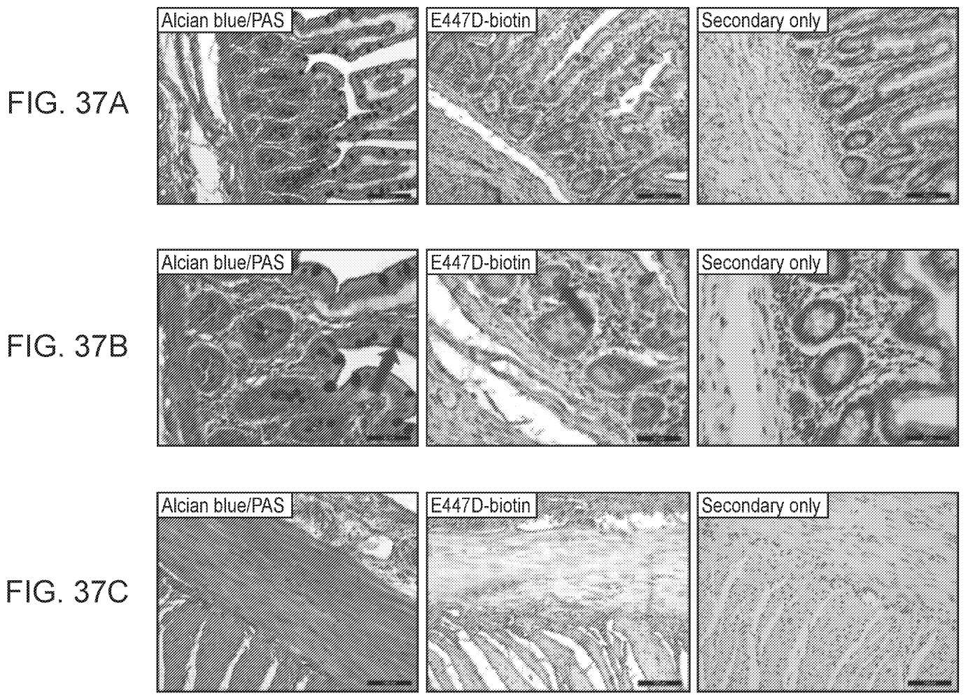

FIG. 37, panels a-c show that StcE E447D can be used to stain tissues for immunohistochemistry. Healthy small intestine jejunum tissue (Novus Biologicals) was stained with alcian blue (pH 2.5)/periodic acid-Schiff stain (Alcian blue/PAS) (Abcam) to visualize acidic (dark purple) and neutral (pink/magenta) glycoproteins as a positive control. Tissues were incubated with 20 μg/mL biotin-StcE E447D (1.89 mol biotin/mol E447D) followed by streptavidin HRP (Abcam) and 3,3′-diaminobenzidine (DAB) chromogen (Abcam) to visualize E447D substrates (brown). The process was repeated without biotin-StcE E447D as a negative control (secondary only). Cell nuclei were counterstained with Hematoxylin(blue) (Abcam). Images were obtained with a Leica DM2000 histology scope (Stanford CSIF) showing (a) intestinal glands and villi at 20× (scale bar:100 μm); (b) intestinal glands and villi at 40× (scale bar: 50 μm); and (c) muscularis externa at 20× (scale bar: 100 μm).

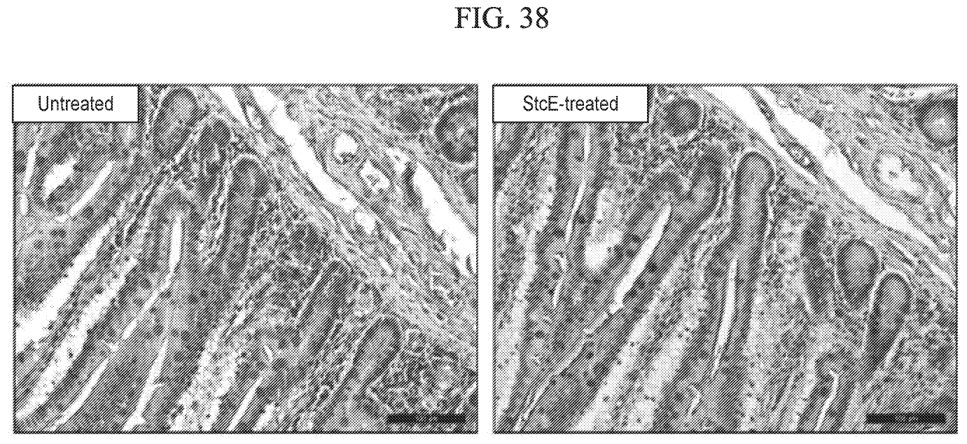

FIG. 38 shows that StcE pretreatment of tissues decreases StcE E447D immunohistochemistry staining Healthy small intestine jejunum tissue (Novus Biologicals) was treated with 10 μg/mL StcE or PBS (untreated control) overnight at room temperature. Tissues were incubated with 20 μg/mL biotin-StcE E447D (1.89 mol biotin/mol E447D) followed by streptavidin HRP (Abcam) and 3,3′-diaminobenzidine (DAB) chromogen (Abcam) to visualize E447D substrates. Cell nuclei were counterstained with Hematoxylin (Abcam). Images were obtained with a Leica DM2000 histology scope (Stanford CSIF) showing decreased DAB signal for the StcE treated sample (scale bar: 100 μm).

Enrichment of Mucin-Domain Glycoproteins from Lysate and Ascites Fluid Using Inactive Point Mutant Mucinases

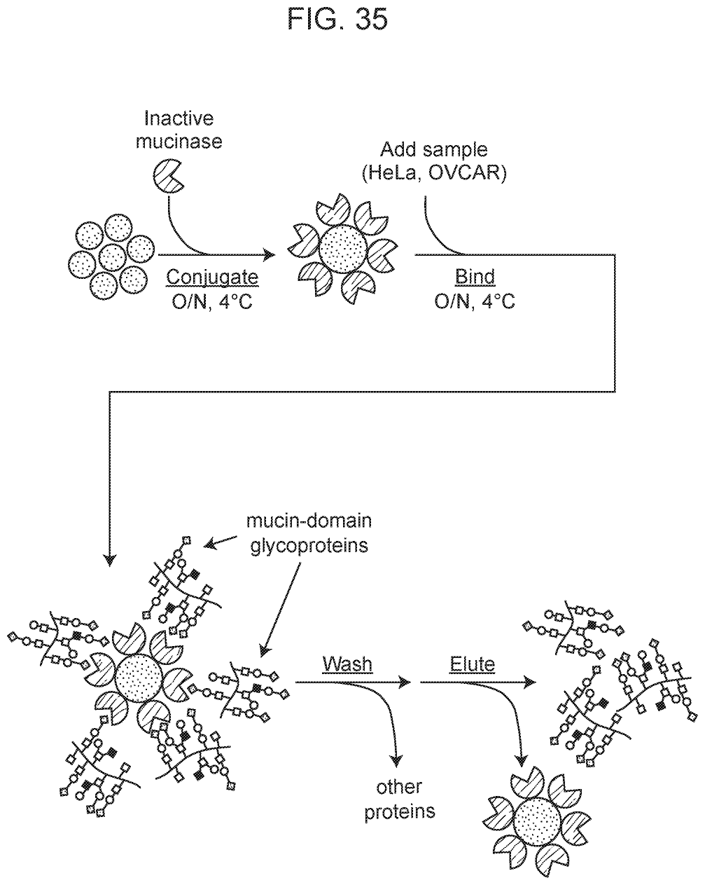

FIG. 35 provides an illustration of enrichment procedure. Inactivated and/or point-mutant mucinases are conjugated to beads overnight at 4° C. Sample (lysate, ascites fluid) is added to the beads and bound overnight at 4° C. Beads are washed three times, and then mucin-domain glycoproteins are eluted by boiling in protein loading buffer. The samples are analyzed by western blot or mass spectrometry.

Enzyme-bead conjugation. Enzymes (˜2 mg in 1 mL) were added to 7 mg of 20 μm POROS AL beads (ThermoFisher Scientific), followed by the addition of 1 μL of 80 mg/mL NaCNBH₃ (Millipore Sigma). After incubation overnight at 4° C., the beads were washed three times with water, and then brought up in Tris-HCl, pH 7, followed by the addition of 1 μL of 80 mg/mL NaCNBH₃. The reaction proceeded for 2-6 hours at room temperature, followed by 3 washes in water. After washing, beads were stored in 1 mL PBS at 4° C. until use. In cases where the enzymes were less concentrated, the amount of beads was decreased proportionally.

Enrichment of mucins from cell lysate and crude cancer patient ascites fluid. Crude lysate and ascites fluid were spun at 18,000×g for 20 min. Clarified samples were subjected to BCA analysis. For control samples, 6 aliquots of 6-30 μL of lysate (6% of enrichment input) were incubated with 2-10 μL of 4× protein loading buffer, boiled for 5 min., and spun at 13,000×g for 2 min. For enriched samples, 6 aliquots of 100-500 μL of lysate or ascites fluid (for a total of 0.5 mg protein input) was incubated with 6 aliquots of 100 μL of conjugated beads (200 μg of enzyme) in 25 mM ETDA overnight at 4° C. After incubation, beads were washed 3 times with 250 μL of PBS in 25 mM ETDA, spun at 8500 rpm, and the supernatant was discarded. To elute, 40 μL of 4× protein loading buffer was added, samples were boiled for 5 min, spun at 13,000×g for 2 min, and frozen until further use.

In-gel digests. Samples (6 per condition) were loaded onto a 4-12% Bis-Tris gel and run at 180 V in MOPS buffer for approximately 1 h. Afterward, gels were stained in AquaStain Protein Gel Stain for 20-30 min, then destained three times in water for 10 min each. Bands (totaling 8) were cut from each lane (6 per condition) for a total of 48 bands per condition. Gel slices were washed once with 200 μL of water, followed by 200 μL of acetonitrile, and equilibrated with 200 μL of 50 mM ammonium bicarbonate (ABC) for 20 min. Gel slices were reduced in 5 mM DTT in 50 mM ABC for 35 min. at 65° C., followed by alkylation in 25 mM IAA in 50 mM ABC for 30 min. at room temperature. The slices were then washed once with 200 μL of 50 mM ABC, followed by two washes with 200 μL of 50:50 acetonitrile:ABC, then dried in a vacuum concentrator for approximately 30 min. The dried slices were resuspended in 0.1 μg trypsin in 50 mM ABC and incubated overnight at 37° C. Afterward, the solution was acidified by the addition of 2.5 μL of formic acid, and incubated for 45 min. Finally, peptides were eluted twice with 100 μL of 70% acetonitrile in water, for 30 min. each. Adjacent band elutions were combined for a total of 400 μL per replicate. Samples were taken to dryness in a vacuum concentrator.

C18 cleanup of gel slices. Samples were reconstituted in 150 μL of solvent A (0.1% formic acid in water) and desalted using a HyperSep C18 96-well filter plate (Thermo Scientific). Briefly, wells were washed with 150 μL of solvent B (80% acetonitrile with 0.1% formic acid) and spun in a table-top centrifuge at 3000×g, followed by equilibration with 150 μL of solvent A. Samples (150 μL) were loaded into the plate 4 times, followed by 3 washes with 150 μL of solvent A. Peptides were eluted three times with 100 μL of solvent B and dried in a vacuum concentrator.

Mass spectrometry. Samples were reconstituted in 8 μL of solvent A and analyzed by online nanoflow LC-MS/MS using an Orbitrap Fusion Tribrid mass spectrometer (Thermo Fisher) coupled to a Dionex Ultimate 3000 HPLC (Thermo Fisher). A portion of the sample (6.5 μL of 8; 80%) was loaded via autosampler onto a C18 nano pre-column using 0.1% formic acid in water (“Solvent A”). For pre-concentration and desalting, the column was washed with 2% ACN and 0.1% formic acid in water (“loading pump solvent”). Subsequently, the C18 nano pre-column was switched in line with the C18 nano separation column (75 μm×250 mm EASYSpray (Thermo Fisher) containing 2 μm C18 beads) for gradient elution. The column was held at 40° C. using a column heater in the EASY-Spray ionization source (Thermo Fisher). The samples were eluted at a constant flow rate of 0.3 μL/min using a 90-minute gradient and a 120-minute instrument method. The gradient profile was as follows (min:% solvent B, 2% formic acid in acetonitrile) 0:3, 3:3, 93:35, 103:42, 104:95,109:95, 110:3, 120:3. The instrument method used an MS1 resolution of 60,000 at FWHM400 m/z, an AGC target of 3e5, and a mass range from 300 to 1,500 m/z. Dynamic exclusion was enabled with a repeat count of 3, repeat duration of 10 s, exclusion duration of 10 s. Only charge states 2-6 were selected for fragmentation. MS2s were generated at top speed for 3 s. HCD was performed on all selected precursor masses with the following parameters: isolation window of 2 m/z, 30% collision energy, orbitrap (resolution of 30,000) detection, and AGC target of 1e4 ions.

Data analysis. Raw files were loaded into MaxQuant and processed using Perseus. Data was log-2 transformed, missing values were imputed based on a normal distribution, and then data was exported from Perseus into excel. Averages for replicates were calculated, along with fold changes and p-values using a two-tailed t-test. Proteins were filtered for those that were enriched in the elution (p<0.05) and then run through an in-house program called STPcalc that determines the ratio of Ser, Thr, and Pro to the entire protein. This program also outputs the fasta files of the enriched proteins, which are then run through the NetOglyc server to determine potential sites of modification. Finally, this output is run through another in-house program called NetOGlycResultsCompiler, which outputs whether or not an enriched protein is a mucin. To be called a mucin, there must be 10 predicted glycosites within a 100 residue stretch.

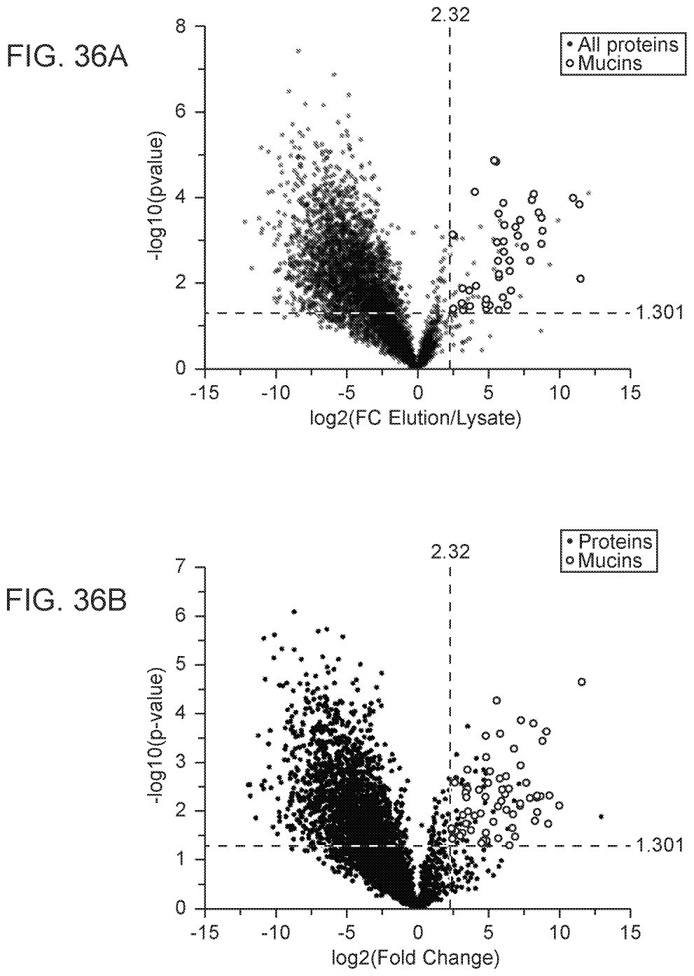

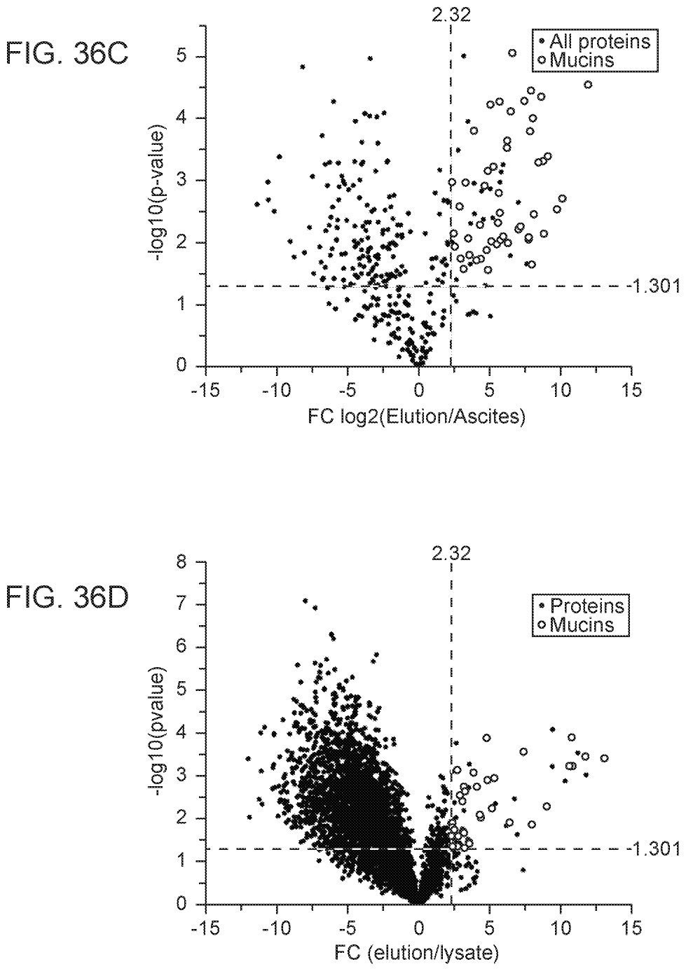

In FIG. 36A, a volcano plot of StcE-enrichment with HeLa lysate is shown. Fold change is shown on the x-axis, and 2.32 indicates >5-fold enrichment of mucins compared to lysate alone. Significance is displayed on the y-axis, where 1.301 designates a p-value of <0.05. Significantly enriched proteins are in the upper-right quadrant, and proteins with a mucin domain are highlighted by enlarged circles.

In FIG. 36B, a volcano plot of StcE-enrichment with OVCAR3 lysate is depicted. Fold change is shown on the x-axis, and 2.32 indicates >5-fold enrichment of mucins compared to lysate alone. Significance is displayed on the y-axis, where 1.301 designates a p-value of <0.05. Significantly enriched proteins are in the upper-right quadrant, and proteins with a mucin domain are highlighted by enlarged circles.

In FIG. 36C, a volcano plot of StcE-enrichment with crude cancer-patient ascites fluid (OC235) is shown. Fold change is shown on the x-axis, and 2.32 indicates >5-fold enrichment of mucins compared to lysate alone. Significance is displayed on the y-axis, where 1.301 designates a p-value of <0.05. Significantly enriched proteins are in the upper-right quadrant, and proteins with a mucin domain are highlighted by enlarged circles.

In FIG. 36D, a volcano plot of BT4244-enrichment with HeLa lysate is depicted. Fold change is shown on the x-axis, and 2.32 indicates >5-fold enrichment of mucins compared to lysate alone. Significance is displayed on the y-axis, where 1.301 designates a p-value of <0.05. Significantly enriched proteins are in the upper-right quadrant, and proteins with a mucin domain are highlighted by enlarged circles.

Notwithstanding the appended claims, the disclosure is also defined by the following clauses:

1. A method comprising:

contacting a sample containing or suspected of containing a mucin-domain glycoprotein comprising a mucin-specific glycan-peptide cleavage motif with a mucin-specific protease that cleaves the cleavage sequence to generate glycopeptides and de-mucinated byproduct; and

analyzing the generated glycopeptides, the de-mucinated byproduct, or both.

2. The method according to clause 1, wherein the mucin-specific glycan-peptide cleavage motif is S/T*-X-S/T, wherein * denotes glycosylation of the S or T residue and X is any amino acid residue or absent.
3. The method according to any of the preceding clauses, wherein the method comprises detecting the presence of the mucin-domain glycoprotein in the sample based on detecting the generated glycopeptides.
4. The method according to any of the preceding clauses, wherein the de-mucinated byproduct comprises de-mucinated cells.
5. The method according to clause 4, wherein the analyzing comprises evaluating a phenotype of the de-mucinated cells.
6. The method according to clauses 4 or 5, further comprising comparing the de-mucinated cells to a control population of cells that are not de-mucinated.
7. The method according to any of the preceding clauses, wherein the mucin-specific protease is a secreted protease of C1 esterase inhibitor (StcE) having at least 90% sequence identity with SEQ ID NO:1.
8. The method according to any one of clauses 1-6, wherein the mucin-specific protease is selected from the group consisting of Pic, ZmpC, BT4244, AM0627, AM0908, AM1514, SmEnhancin, and VIBHAR2194.
9. The method according to any one of clauses 1-6, wherein the mucin-specific protease is AM0627 or BT4244.
10. The method according to any of the preceding clauses, wherein the sample is an acellular proteinaceous sample or a cellular sample.
11. The method according to clause 10, wherein the cellular sample is prepared from a cell culture or a biopsy.
12. The method according to clause 11, wherein the cell culture comprises cultured cancer cells.
13. The method according to clause 11, wherein the biopsy is a cancer biopsy.
14. The method according to any of the preceding clauses, wherein the method further comprises enriching the sample for glycopeptides or isolating glycopeptides from the sample.
15. The method according to clause 14, wherein sample is enriched for the generated glycopeptides or the generated glycopeptides are isolated prior to the analyzing.
16. The method according to any of the preceding clauses, wherein the analyzing comprises mass spectrometry.
17. The method according to any of the preceding clauses, wherein the method further comprises determining the amino acid sequence of at least a portion of a glycopeptide of the generated glycopeptides.
18. The method according to clause 17, wherein the method further comprises identifying one or more glycosites of the glycopeptide.
19. The method according to any of the preceding clauses, wherein the method does not comprise releasing glycans from the generated glycopeptides.
20. A method comprising:

contacting a cellular sample with a mucin-specific protease to generate a population of mucin-domain cleaved glycopeptides; and

analyzing the population of mucin-domain cleaved glycopeptides using mass spectrometry to produce a mucin-domain cleaved glycosignature.

21. The method according to clause 20, wherein the mucin-specific protease is a secreted protease of C1 esterase inhibitor (StcE) having at least 90% sequence identity with SEQ ID NO:1
22. The method according to clause 20, wherein the mucin-specific protease is selected from the group consisting of Pic, ZmpC, BT4244, AM0627, AM0908, AM1514, SmEnhancin, and VIBHAR2194.
23. The method according to clause 20, wherein the mucin-specific protease is AM0627 or BT4244.
24. The method according to any of clauses 20 to 23, wherein the method further comprises isolating the population of cleaved glycopeptides prior to the analyzing.
25. The method according to any of clauses 20 to 24, wherein the method further comprises analyzing a population of de-mucinated cells generated during the contacting.
26. The method according to clause 25, wherein the method further comprises isolating the population of cells prior to the analyzing.
27. The method according to any of clauses 20 to 26, wherein the method further comprises deglycosylating glycoproteins of the cellular sample.
28. The method according to any of clauses 20 to 27, wherein the method further comprises analyzing a population of deglycosylated glycoproteins using mass spectrometry.
29. The method according to any of clauses 20 to 28, wherein the method further comprises analyzing a population of glycopeptides from the cellular sample using mass spectrometry to produce a non-mucin-cleaved glycosignature.
30. The method according to clause 29, wherein the method further comprises comparing the mucin-cleaved glycosignature to the non-mucin-cleaved glycosignature.
31. A method for detecting a condition characterized by aberrant glycosylation in a subject, the method comprising:

determining a mucin-domain cleaved glycosignature from a biological sample from said subject according to the method of any of clauses 20 to 30; and

comparing the mucin-domain cleaved glycosignature to a healthy reference or control mucin-domain cleaved glycosignature to detect the condition.

32. The method according to clause 31, wherein the condition is cancer.
33. A method of treating a subject for a cancer, the method comprising:

performing, or having performed, the method according to clause 32 to detect whether a subject has a cancer characterized by aberrant glycosylation; and

treating the subject with a mucin-domain directed therapy when the subject is identified as having the cancer characterized by aberrant glycosylation.

34. The method according to clause 33, wherein the mucin-domain directed therapy comprises a mucin-domain glycoprotein-specific antibody.
35. The method according to clauses 33 or 34, wherein the mucin-domain directed therapy comprises a mucin-domain glycoprotein-specific chimeric antigen receptor (CAR).
36. The method according to any of clauses 33 to 35, wherein the mucin-domain directed therapy comprises an anti-mucin vaccine.
37. The method according to any of clauses 33 to 36, wherein the mucin-domain directed therapy comprises a mucin inhibitor.
38. A method of identifying a receptor as mucin-domain glycoprotein-specific, the method comprising:
contacting a cellular sample with a mucin-specific protease to generate a de-mucinated cellular sample; and
assessing binding of the receptor with the cellular sample and the de-mucinated cellular sample, wherein decreased binding of the receptor to cells of the de-mucinated cellular sample as compared to cells of the cellular sample identifies the receptor as a mucin-domain glycoprotein-specific receptor.
39. The method according to clause 38, wherein the mucin-specific protease is a secreted protease of C1 esterase inhibitor (StcE) having at least 90% sequence identity with SEQ ID NO:1
40. The method according to clause 39, wherein the mucin-specific protease is selected from the group consisting of Pic, ZmpC, BT4244, AM0627, AM0908, AM1514, SmEnhancin, and VIBHAR2194.
41. The method according to clause 39, wherein the mucin-specific protease is AM0627 or BT4244.
42. The method according to any of clauses 38 to 41, wherein the receptor is an orphan receptor.
43. The method according to clauses 38 to 42, wherein the method further comprises assessing binding of a control receptor known to be mucin-domain glycoprotein-specific.
44. The method according to any of clauses 38 to 43, wherein the method further comprises assessing binding of a control receptor known not to be mucin-domain glycoprotein-specific.
45. A method comprising:
contacting a sample with a catalytically inactive mucin-specific protease that binds a mucin-domain glycoprotein present in the sample; and
separating the bound mucin-specific protease from at least a portion of the sample to isolate, enrich or deplete the mucin-domain glycoprotein from or in the sample.
46. The method according to clause 45, wherein the catalytically inactive mucin-specific protease is: a mutant that lacks protease activity, in the presence of a protease inhibitor, or both.
47. The method according to clauses 45 or 46, wherein the mucin-specific protease is a secreted protease of C1 esterase inhibitor (StcE) having at least 90% sequence identity with SEQ ID NO:1
48. The method according to clause 47, wherein the StcE has 100% sequence identity with SEQ ID NO:1.
49. The method according to clause 47, wherein the StcE is a recombinant StcE variant having less than 100% sequence identity with SEQ ID NO:1.
50. The method according to clause 49, wherein recombinant StcE variant comprises a E447D mutation.
51. The method according to clauses 45 or 46, wherein the mucin-specific protease is selected from the group consisting of Pic, ZmpC, BT4244, AM0627, AM0908, AM1514, SmEnhancin, and VIBHAR2194.
52. The method according to clauses 45 or 46, wherein the mucin-specific protease is BT4244 or AM0627.
53. The method according to clause 52, wherein the mucin-specific protease is a recombinant AM0627 variant comprising a substitution at amino acid position 326 or a recombinant BT4244 variant comprising a substitution at amino acid position 575.
54. The method according to clause 53, wherein the substitution at amino acid position E326 is E326A.
55. The method according to clause 53, wherein the substitution at amino acid position E575 is E575A.
56. The method according to any one of clauses 45 to 55, wherein the mucin-specific protease is bound to a solid support.
57. The method according to clause 56, wherein the method comprises contacting the sample with the solid support to bind the mucin-domain glycoprotein and extracting the solid support from the sample to isolate the mucin-domain glycoprotein.
58. The method according to clause 56, wherein the method comprises contacting the sample with the solid support to bind the mucin-domain glycoprotein and retaining the solid support to enrich the sample for the mucin-domain glycoprotein.
59. The method according to any of clauses 45 to 58, wherein the mucin-domain glycoprotein isolated, enriched, or depleted is an intact mucin-domain glycoprotein.
60. A kit comprising:
one or more containers comprising a mucin-specific protease.
61. The kit according to clause 60, wherein the mucin-specific protease is a secreted protease of C1 esterase inhibitor (StcE) having at least 90% sequence identity with SEQ ID NO:1 or a nucleic acid encoding the StcE.
62. The kit according to clause 60, wherein the StcE is a recombinant StcE variant having less than 100% sequence identity with SEQ ID NO:1.
63. The kit according to clause 60, wherein the mucin-specific protease is selected from the group consisting of Pic, ZmpC, BT4244, AM0627, AM0908, AM1514, SmEnhancin, and VIBHAR2194.
64. The kit according to clause 60, wherein the mucin-specific protease is AM0627 or VIBHAR2194.
65. The kit according to any one of clauses 60-64, wherein the mucin-specific protease is conjugated to a detectable label.
66. The kit according to clause 65, wherein the detectable label comprises a fluorescent molecule, luminescent molecule, light-scattering molecule, or a quantum dot.
67. The kit according to any of clauses 60 to 66, wherein the mucin-specific protease is catalytically inactive, the kit comprises a protease inhibitor, or both.
68. The kit according to any of clauses 60 to 66, wherein the kit comprises the mucin-specific protease in a dry composition.
69. The kit according to clause 68, wherein the mucin-specific protease is lyophilized.
70. The kit according to any of any of clauses 60 to 69, wherein the mucin-specific protease is attached to a solid support.
71. The kit according to any of clauses 60 to 61, wherein the kit comprises a plasmid comprising a nucleic acid encoding the mucin-specific protease.
72. The kit according to any of clauses 60 to 70, further comprising a buffer in which the mucin-specific protease is active.
73. The kit according to any of clauses 60 to 72, further comprising a deglycosylase.
74. The kit according to clause 73, wherein the deglycosylase is PNGase F.
75. The kit according to any of clauses 60 to 74, further comprising a protease.
76. The kit according to clause 75, wherein the protease is trypsin.
77. The kit according to any of clauses 60 to 76, further comprising one or more purification devices and/or reagents.
78. A method comprising:
contacting a sample with a catalytically inactive mucin-specific protease that binds a mucin-domain glycoprotein present in the sample;
detecting binding of the catalytically inactive mucin-specific protease to the sample.
79. The method according to clause 78, wherein the catalytically inactive mucin-specific protease is a variant of a mucin-specific protease selected from the group consisting of StcE, Pic, ZmpC, BT4244, AM0627, AM0908, AM1514, SmEnhancin, and VIBHAR2194.
80. The method according to clause 78, wherein the mucin-specific protease is StcE, BT4244, or AM0627.
81. The method according to clause 80, wherein the catalytically inactive mucin-specific protease comprises a sequence of StcE comprising the substitution E447D.
82. The method according to clause 80, wherein the catalytically inactive mucin-specific protease comprises a sequence of BT4244 comprising the substitution E575A.
83. The method according to clause 80, wherein the catalytically inactive mucin-specific protease comprises a sequence of AM0627 comprising the substitution E326A.
84. The method according to any of clauses 78-83, wherein the sample is a tissue sample.
85. The method according to clause 84, wherein the tissue sample is a small intestinal tissue sample.
86. The method according to any of clauses 80-85, wherein the catalytically inactive mucin-specific protease comprises a detectable label.
87. The method according to clause 86, wherein the detectable label is a fluorescent molecule, luminescent molecule, light-scattering molecule, or a quantum dot.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

TABLE 1
MUCINASE SEQUENCES

| SEQ | | |
|---|---|---|
| ID | | |
| NO: | Sequence | Description |
| 1 | ADNNSAIYFNTSQPINDLQGSLAAEVKFAQSQILPAHPKEGDSQPH | StcE amino |
| | LTSLRKSLLLVRPVKADDKTPVQVEARDDNNKILGTLTLYPPSSLP | acid sequence |
| | DTIYHLDGVPEGGIDFTPHNGTKKIINTVAEVNKLSDASGSSIHSH | |
| | LTNNALVEIHTANGRWVRDIYLPQGPDLEGKMVRFVSSAGYSSTVF | |
| | YGDRKVTLSVGNTLLFKYVNGQWFRSGELENNRITYAQHIWSAELP | |
| | AHWIVPGLNLVIKQGNLSGRLNDIKIGAPGELLLHTIDIGMLTTPR | |
| | DRFDFAKDKEAHREYFQTIPVSRMIVNNYAPLHLKEVMLPTGELLT | |
| | DMDPGNGGWHSGTMRQRIGKELVSHGIDNANYGLNSTAGLGENSHP | |
| | YVVAQLAAHNSRGNYANGIQVHGGSGGGGIVTLDSTLGNEFSHEVG | |
| | HNYGLGHYVDGFKGSVHRSAENNNSTWGWDGDKKRFIPNFYPSQTN | |
| | EKSCLNNQCQEPFDGHKFGFDAMAGGSPFSAANRFTMYTPNSSAII | |
| | QRFFENKAVFDSRSSTGFSKWNADTQEMEPYEHTIDRAEQITASVN | |
| | ELSESKMAELMAEYAVVKVHMWNGNWTRNIYIPTASADNRGSILTI | |
| | NHEAGYNSYLFINGDEKVVSQGYKKSFVSDGQFWKERDVVDTREAR | |
| | KPEQFGVPVTTLVGYYDPEGTLSSYIYPAMYGAYGFTYSDDSQNLS | |
| | DNDCQLQVDTKEGQLRFRLANHRANNTVMNKFHINVPTESQPTQAT | |
| | LVCNNKILDTKSLTPAPEGLTYTVNGQALPAKENEGCIVSVNSGKR | |
| | YCLPVGQRSGYSLPDWIVGQEVYVDSGAKAKVLLSDWDNLSYNRIG | |
| | EFVGNVNPADMKKVKAWNGQYLDFSKPRSMRVVYK | |
| 2 | ATGAACACTAAAATGAATGAGAGATGGAGAACACCGATGAAATTAA | StcE |
| | AGTATCTGTCATGTACGATCCTTGCCCCTCTGGCGATTGGGGTATT | nucleotide |
| | TTCTGCAACAGCTGCTGATAATAATTCAGCCATTTATTTCAATACC | sequence |
| | TCCCAGCCTATAAATGATCTGCAGGGTTCGTTGGCCGCAGAGGTGA | |
| | AATTTGCACAAAGCCAGATTTTACCCGCCCATCCTAAAGAAGGGGA | |
| | TAGTCAACCACATCTGACCAGCCTGCGGAAAAGTCTGCTGCTTGTC | |
| | CGTCCGGTGAAAGCTGATGATAAAACACCTGTTCAGGTGGAAGCCC | |
| | GCGATGATAATAATAAAATTCTCGGTACGTTAACCCTTTATCCTCC | |
| | TTCATCACTACCGGATACAATCTACCATCTGGATGGTGTTCCGGAA | |
| | GGTGGTATCGATTTCACACCTCATAATGGAACGAAAAAGATCATTA | |
| | ATACGGTGGCTGAAGTAAACAAACTCAGTGATGCCAGCGGGAGTTC | |
| | TATTCATAGCCATCTAACAAATAATGCACTGGTGGAGATCCATACT | |
| | GCAAATGGTCGTTGGGTAAGAGACATTTATCTGCCGCAGGGACCCG | |
| | ACCTTGAAGGTAAGATGGTTCGCTTTGTTTCGTCTGCAGGCTATAG | |
| | TTCAACGGTTTTTTATGGTGATCGAAAAGTCACACTCTCGGTGGGT | |
| | AACACTCTTCTGTTCAAATATGTAAATGGTCAGTGGTTCCGCTCCG | |
| | GTGAACTGGAGAATAATCGAATCACTTATGCTCAGCATATTTGGAG | |
| | TGCTGAACTGCCTGCGCACTGGATCGTGCCTGGTTTAAACTTGGTG | |
| | ATTAAACAGGGCAATCTGAGCGGTCGCCTAAATGATATCAAGATTG | |
| | GAGCACCGGGTGAGCTGTTGTTGCATACAATTGATATCGGGATGTT | |
| | GACCACTCCCCGGGATCGCTTTGATTTTGCCAAAGACAAAGAAGCA | |
| | CATAGGGAATATTTCCAGACCATTCCTGTAAGTCGTATGATTGTTA | |
| | ATAATTATGCGCCTCTACACCTAAAGGAAGTTATGTTACCAACCGG | |
| | AGAGTTATTGACAGATATGGATCCAGGAAATGGTGGGTGGCATAGT | |
| | GGTACAATGCGTCAAAGAATAGGTAAAGAATTGGTTTCGCATGGCA | |
| | TTGATAATGCTAACTATGGTTTAAATAGTACCGCAGGCTTAGGGGA | |
| | GAATAGTCATCCATATGTAGTTGCGCAATTAGCGGCACATAATAGC | |
| | CGCGGTAATTATGCTAATGGCATCCAGGTTCATGGTGGCTCCGGAG | |
| | GTGGGGGAATTGTTACTTTAGATTCCACATTGGGGAATGAGTTCAG | |
| | TCATGAAGTTGGTCATAATTATGGTCTTGGTCATTATGTAGATGGT | |
| | TTCAAGGGTTCTGTACATCGTAGTGCAGAAAATAACAACTCAACTT | |
| | GGGGATGGGATGGTGATAAAAAACGGTTTATTCCTAACTTTTATCC | |
| | GTCTCAAACAAATGAAAAGAGTTGTCTGAATAATCAGTGTCAAGAA | |
| | CCGTTTGATGGACACAAATTTGGTTTTGACGCCATGGCGGGAGGCA | |
| | GCCCTTTCTCTGCTGCAAACCGTTTCACAATGTATACTCCGAATTC | |
| | ATCGGCTATCATCCAGCGTTTTTTTGAAAATAAAGCTGTGTTCGAT | |
| | AGCCGTTCCTCCACCGGCTTCAGCAAGTGGAATGCAGATACGCAGG | |
| | AAATGGAACCGTATGAACACACCATTGACCGTGCGGAGCAGATTAC | |
| | GGCTTCAGTCAATGAGCTAAGTGAAAGCAAAATGGCTGAGCTGATG | |
| | GCAGAGTACGCTGTCGTCAAAGTGCATATGTGGAACGGTAACTGGA | |
| | CAAGAAACATCTATATCCCTACAGCCTCCGCAGATAATAGAGGCAG | |
| | TATCCTGACCATCAACCATGAGGCCGGTTATAATAGTTATCTGTTT | |
| | ATAAATGGTGACGAAAAGGTCGTTTCCCAGGGGTATAAAAAGAGCT | |
| | TTGTTTCCGATGGTCAGTTCTGGAAAGAACGTGATGTGGTTGATAC | |
| | TCGTGAAGCGCGTAAGCCAGAGCAGTTTGGTGTTCCTGTGACGACC | |
| | CTGGTGGGGTATTACGATCCGGAAGGCACGCTGTCAAGCTACATCT | |
| | ATCCTGCGATGTATGGTGCCTATGGCTTCACTTATTCCGATGATAG | |
| | TCAGAATCTATCCGATAACGACTGCCAGCTGCAGGTGGATACGAAA | |
| | GAAGGGCAGTTGCGATTCAGACTGGCTAATCACCGGGCTAACAACA | |
| | CTGTAATGAATAAGTTCCATATTAACGTGCCAACAGAAAGTCAGCC | |
| | CACACAGGCCACATTGGTTTGCAATAACAAGATACTGGATACCAAA | |
| | TCGCTCACACCTGCGCCAGAAGGACTTACCTATACTGTAAATGGGC | |
| | AGGCACTTCCAGCAAAAGAAAACGAGGGATGCATCGTGTCCGTGAA | |
| | TTCAGGTAAACGTTACTGTTTGCCGGTTGGTCAACGGTCAGGATAT | |
| | AGCCTTCCTGACTGGATTGTTGGGCAGGAAGTCTATGTCGACAGCG | |
| | GGGCTAAAGCGAAAGTGCTGCTTTCTGACTGGGATAACCTGTCCTA | |
| | TAACAGGATTGGTGAGTTTGTAGGTAATGTGAACCCAGCTGATATG | |
| | AAAAAAGTTAAAGCCTGGAACGGACAGTATTTGGACTTCAGTAAAC | |
| | CTAGGTCAATGAGGGTTGTATATAAATAA | |
| 17 | MNKVYSLKYCPVTGGLIAVSELARRVIKKTCRRLTHILLAGIPAIC | Pic from |
| | LCYSQISQAGIVRSDIAYQIYRDFAENKGLFVPGANDIPVYDKDGK | Escherichia coli |
| | LVGRLGKAPMADFSSVSSNGVATLVSPQYIVSVKHNGGYRSVSFGN | 042 |
| | GKNTYSLVDRNNHPSIDFHAPRLNKLVTEVIPSAVTSEGTKANAYK | |
| | YTERYTAFYRVGSGTQYTKDKDGNLVKVAGGYAFKTGGTTGVPLIS | |
| | DATIVSNPGQTYNPVNGPLPDYGAPGDSGSPLFAYDKQQKKWVIVA | |
| | VLRAYAGINGATNWWNVIPTDYLNQVMQDDFDAPVDFVSGLGPLNW | |
| | TYDKTSGTGTLSQGSKNWTMHGQKDNDLNAGKNLVFSGQNGAIILK | |
| | DSVTQGAGYLEFKDSYTVSAESGKTWTGAGIITDKGTNVTWKVNGV | |
| | AGDNLHKLGEGTLTINGTGVNPGGLKTGDGIVVLNQQADTAGNIQA | |
| | FSSVNLASGRPTVVLGDARQVNPDNISWGYRGGKLDLNGNAVTFTR | |
| | LQAADYGAVITNNAQQKSQLLLDLKAQDTNVSEPTIGNISPFGGTG | |
| | TPGNLYSMILNSQTRFYILKSASYGNTLWGNSLNDPAQWEFVGMDK | |
| | NKAVQTVKDRILAGRAKQPVIFHGQLTGNMDVAIPQVPGGRKVIFD | |
| | GSVNLPEGTLSQDSGTLIFQGHPVIHASISGSAPVSLNQKDWENRQ | |
| | FTMKTLSLKDADFHLSRNASLNSDIKSDNSHITLGSDRAFVDKNDG | |
| | TGNYVIPEEGTSVPDTVNDRSQYEGNITLNHNSALDIGSRFTGGID | |
| | AYDSAVSITSPDVLLTAPGAFAGSSLTVHDGGHLTALNGLFSDGHI | |
| | QAGKNGKITLSGTPVKDTANQYAPAVYLTDGYDLTGDNAALEITRG | |
| | AHASGDIHASAASTVTIGSDTPAELASAETAASAFAGSLLEGYNAA | |
| | FNGAITGGRADVSMHNALWTLGGDSAIHSLTVRNSRISSEGDRTFR | |
| | TLTVNKLDATGSDFVLRTDLKNADKINVTEKATGSDNSLNVSFMNN | |
| | PAQGQALNIPLVTAPAGTSAEMFKAGTRVTGFSRVTPTLHVDTSGG | |
| | NTKWILDGFKAEADKAAAAKADSFMNAGYKNFMTEVNNLNKRMGDL | |
| | RDTNGDAGAWARIMSGAGSADGGYSDNYTHVQVGFDKKHELDGVDL | |
| | FTGVTMTYTDSSADSHAFSGKTKSVGGGLYASALFESGAYIDLIGK | |
| | YIHHDNDYTGNFASLGTKHYNTHSWYAGAETGYRYHLTEDTFIEPQ | |
| | AELVYGAVSGKTFRWKDGDMDLSMKNRDFSPLVGRTGVELGKTFSG | |
| | KDWSVTARAGTSWQFDLLNNGETVLRDASGEKRIKGEKDSRNILFN | |
| | VGMNAQIKDNMRFGLEFEKSAFGKYNVDNAVNANFRYMF | |
| 18 | LSAYNSQLSIGVGEHLPEPLKIEGYQYIGYIKTKKQDNTELSRTVD | ZmpC from |
| | GKYSAQRDSQPNSTKTSDVVHSADLEWNQGQGKVSLQGEASGDDGL | Streptococcus |
| | SEKSSIAADNLSSNDSFASQVEQNPDHKGESVVRPTVPEQGNPVSA | pneumoniae |
| | TTVQSAEEEVLATTNDRPEYKLPLETKGTQEPGHEGEAAVREDLPV | |
| | YTKPLETKGTQGPGHEGEAAVREEEPAYTEPLATKGTQEPGHEGKA | |
| | TVREETLEYFEPVATKGTQEPEHEGEAAVEEELPALEVTTRNRFLI | |
| | QNIPYTTEEIQDPTLLKNRRKIERQGQAGTRTIQYEDYIVNGNVVE | |
| | TKEVSRTEVAPVNEVVKVGTLVKVKPTVEITNLTKVENKKSITVSY | |
| | NLIDTTSAYVSAKTQVFHGDKLVKEVDIENPAKEQVISGLDYYTPY | |
| | TVKTHLTYNLGENNEENTETSTQDFQLEYKKIEIKDIDSVELYGKE | |
| | NDRYRRYLSLSEAPTDTAKYFVKVKSDREKEMYLPVKSIFENTDGT | |
| | YKVTVAVDQLVEEGTDGYKDDYTFTVAKSKAEQPGVYTSFKQLVTA | |
| | MQSNLSGVYTLASDMTADEVSLGDKQTSYLTGAFTGSLIGSDGTKS | |
| | YAIYDLKKPLFDTLNGATVRDLDIKTVSADSKENVAALAKAANSAN | |
| | INNVAVEGKISGAKSVAGLVASATNTVIENSSFTGKLIANHQDSNK | |
| | NDTGGIVGNITGNSSRVNKVRVDALISTNARNNNQTAGGIVGRLEN | |
| | GALISNSVATGEIRNGQGYSRVGGIVGSTWQNGRVNNVVSNVDVGD | |
| | GYVITGDQYAAADVKNASTSVDNRKADRFATKLSKDQIDAKVADYG | |
| | ITVTLDDTGQDLKRNLREVDYTRLNKAEAERKVAYSNIEKLMPFYN | |
| | KDLVVHYGNKVATTDKLYTTELLDVVPMKDDEVVTDINNKKNSINK | |
| | VMLHFKDNTVEYLDVTFKENFINSQVIEYNVTGKEYIFTPEAFVSD | |
| | YTAITNNVLSDLQNVTLNSEATKKVLGAANDAALDNLYLDRQULEV | |
| | KANIAEHLRKVLAMDKSINTTGDGVVEYVSEKIKNNKEAFMLGLTY | |
| | MNRWYDINYGKMNTKDLSTYKFDFNGNNETSTLDTIVALGNSGLDN | |
| | LRASNTVGLYANKLASVKGEDSVFDFVEAYRKLFLPNKTNNEWFKE | |
| | NTKAYIVEMKSDIAEVREKQESPTADRKYSLGVYDRISAPSWGHKS | |
| | MLLPLLTLPEESVYISSNMSTLAFGSYERYRDSVDGVILSGDALRT | |
| | YVRNRVDIAAKRHRDHYDIWYNLLDSASKEKLFRSVIVYDGFNVKD | |
| | ETGRTYWARLTDKNIGSIKEFFGPVGKWYEYNSSAGAYANGSLTHF | |
| | VLDRLLDAYGTSVYTHEMVHNSDSAIYFEGNGRREGLGAELYALGL | |
| | LQSVDSVNSHILALNTLYKAEKDDLNRLHTYNPVERFDSDEALQSY | |
| | MHGSYDVMYTLDAMEAKAILAQNNDVKKKWFRKIENYYVRDTRHNK | |
| | DTHAGNKVRPLTDEEVANLTSLNSLIDNDIINRRSYDDSREYKRNG | |
| | YYTISMFSPVYAALSNSKGAPGDIMERMAYELLAEKGYHKGFLPYV | |
| | SNQYGAEAFASGSKTESSWHGRDVALVTDDLVFKKVENGEYSSWAD | |
| | FKKAMFKQRIDKQDNLKPITIQYELGNPNSTKEVTITTAAQMQQLI | |
| | NEAAAKDITNIDRATSHTPASWVHLLKQKIYNAYLRTTDDFRNSIY | |
| | K | |
| 19 | KDTEKSIINSSFSISEEYLIQNLDKSSTSVQIPINTSMELAQWSVS | BT4244 from |
| | YEANWLQCSKQKTAAEGTFLRITVNENTGETKRTANIKVTSTTATY | Bacteroides |
| | TITVNQYAKGEVIVEGDIKVTPTGGKASEHQEGQDIENTYDGKFST | thetaiotaomicron |
| | DGAAPFHTPWGQSAKFPVTLEYYFKGDTEIDYLIYYTRSGNGNEGK | |
| | VKVYTTTNPDRSDYTLQGEYDFKEQNAPSKVSFSEGIKATGIKFEV | |
| | LSGLGDFVSCDEMEFYKTNTDKTLDKQLLTVFIDITCTEIKNNVTN | |
| | EQIQALPDYFVRIAEAVRDNTYDKWEKEFRIRSYEPYSNIAEWADK | |
| | LMTKKYSDLDNPTGISVKAGDDIIVLVGDTYGQNISMQCIWETGFE | |
| | YKQTASSGDVYMLNPGVNKLTMKGEGQLFVMYNTELTSNTAKPIKI | |
| | HIPLGSGTVNGFFDLKEHKTDEKYAELLKKSTHKYFCIRGEKIMFY | |
| | FHRNKLLEYVPNNILSAIHLWDNIVGWQQELMGIDDVRPSQVNNHL | |
| | FAISPEGSYMVVASDYQIGFVYTYLGNILLEDNVMAAEDNAWGPAH | |
| | EIGHVHQAAINWASSTESSNNLFSNFIIYKLGKYKSRGNGLGSVAT | |
| | ARYANGQAWYNMGDATHQNEDFETHMRMNWQLWIYYHRCEYKTDEV | |
| | VQTLFKLMREVNMFEGEDPGKKQLEFAKMASKAANQNLTDFFEMWG | |
| | FFEPVNTTIEQYGTYKYYVSDAMIREAKEYMAQFPAPKHAFQYIED | |
| | RKKSEFPSNDYRYSAVGDVGYYTQFKENQKITKAITAELAGRKVSI | |
| | QNGDEAVAILLRENDENGKLLYESTFITILIPSSILMVNAKLYAVQ | |
| | ADGKRILL | |
| 20 | ANTPEHIGNDLKLFKDSSCTSLKPDVKNTSAFQSDAMKELATKILA | AM0627 from |
| | GHYKPDYLYAEYRALPSPRQTGKNLRIGDGESKYDNMTGVYLEKGR | Akkermansia |
| | HVVLVGKTEGQEISLLLPNLMRKPAEGVQPTKDPNGWGLHKKQIPL | muciniphila |
| | KEGINIIDVETPANAYISYFTEDAGKAPKIPVHFVTGKANGYFDTT | |
| | RGDTNKDWVRLLDQAVSPIMDARGKYIQVAYPVEFLKKFTKDRGFE | |
| | LINAYDKLIGIQYQLMGLDKYGKIPENRVLARVNFNYYMFRDGDGV | |
| | AYLGNDGTMRMVTDPENVLKGDACWGFSHEVGHVMQMRPMTWGGMT | |
| | EVSNNIFSLQAAAKTGNESRLKRQGSYDKARKEIIEGEIAYLQSKD | |
| | VENKLVPLWQLHLYFTKNGHPDFYPDVMEYLRNNAGNYGGNDTVKY | |
| | QULFVKACCDVTKTDLTDFFEKWGFEKPGKFHIGDYAQYDENVTPE | |
| | MVEETKKWIAGKGYPKPETDIFELSE | |
| 21 | KYPSLDVPEDILLHVKEAASSQSPYSGNHSVKQAVDGSMESANWHV | AM0908 from |
| | PGVHHVEGEFVFEVPETIHYIIFSGANFNEIAVSAMSGSSWKDLGK | Akkermansia |
| | EDIGGSRMIRFKKPLQKVRKIRLTVDYPEGSSPSFTVREISFYKRV | muciniphila |
| | ESALNRKLLKVEKDTSCSSINPRCTLTDLKALPEFLQMIAKKIKSG | |
| | DYEDKEFRIASYKAYSHPEFAAKVRNINALNKEDNPTGIVAEKGDE | |
| | ILVFVGPTHGEDIGLASVSPAGIESSSYPLNEGVNKIRINRSGLLY | |
| | VMYHTDISPPKKPITVHIPVGSGIVNGYEDVTRHTDKDWKRMISNA | |
| | PHSMFDIVGRNSMMILHTKYLKDYSPDSITKSVRVWDESVKAMVVK | |
| | IMGFDKYPQPHNNRQLGVSVEGGAHMFATVVYYCGYSIGDQGNTLK | |
| | NEVLAPGVLQGNRLWGIGHEIGHCYQHPFNWRSMSESSNNFFAQLI | |
| | LDQVTNAINGNEQASDMENPCKYLLSEAVKGMPFHDLNGWAKWGFA | |
| | QYSFYLYFHKLGINPEFYPRLFESLRRKPLSRQAYEVSEAHLALYE | |
| | RICNISRTDFTDDFEIFNWFVPIDRKGHQYGDYSFKMTEEMARASK | |
| | ARIAAKRYPKPKFRIAFLHQHGKTVNLWGQNLHGSQLNGYWTKYKQ | |
| | NAKLSPSVSASKKDNMIIVRNGENAAAFCVVTNGKVVGYYDRQKFD | |
| | VSGVEWNDTSKVYAIPIQTAEPYKLIYAAGRS | |
| 22 | QDNLAKRKAAQELTAERKLKEKKAREAAEKQRIKREREIREKKEKE | AM1514 from |
| | RLAAQKAYEEAQEEKARQAAEAARKLQEQAEREEREKRRREELERR | Akkermansia |
| | EREEEARRQEEDTPVEEEPEPEGREPQPVKNRMPELSVYSIPCRDD | muciniphila |
| | IQTEKDKLLETWSWDKAEKMEGMEEFPTGSSPWKKGKDAGRMQALL | |
| | EKCREWKDAKLASLKACPAAKDFPGVPENGAQTVRRTVEIDSNIGG | |
| | WHSTGLYAPPGAEISCSLSGAPKDGSISVRIGCHTDSLHKLDEWKR | |
| | VPEITMQVPAGRGRVKMVNPMGGLVYVNVGQRPRRGKVEKVQISGA | |
| | VPSPLEVMGKTTPEQWAEQLENTKAPWGEIRMPRLIVTMPVEQLKQ | |
| | CPDVQKTAEFLQKNMALQDWIMGWDTKPDRLHHPMRFVVDRQISAG | |
| | AGHSGYPAMATKDWTNSIATGSIIHSGSWGLWHELGHNHQSPPFTM | |
| | EGQIINSVNIFSMVCEVMGTGKDFESCWGGGMGPYGMSAEMKKYFS | |
| | GTQTYNEAPNKVQLFFVVVELMYYLGFDAFRQVALQFHDKPYDNGE | |
| | LSDEKKWEWVMNAFSKVTGKNMGPFFKIWRTPVSERATGRMKDLPA | |
| | WLPSKDYPACYTAEE | |
| 23 | MPTITKTQSLYTLTRPVWLDAAGMSKGIDHDRQHLGIILAAGQVLR | SmEnhancin from |
| | VRQTNAAFTGKLTLRLLNDDSKTEAEFSVGASWVEAQVNAVSVPFI | Serratia |
| | DTPYGQGEPALEFEYPDTAKALPVYRRGENEAAFFARWDDRDAEFA | marcescens |
| | LVDAEYAVLLVPKISKPALKSLGEAKNIDGLIAYYDRIFTFYNALT | |
| | GVSFEAEHESDRNIRNRYFMKADKHGAGAAYYGGRWTAETSSSVSS | |
| | FVVLTPKDSNWGSLHEIAHGYQGHFMGDKYFSTGEMWNNIYAACYQ | |
| | DVMLGERKYQEGWLYNYGNAAGVEKKIVDLIAQGTPLNQWDLRSKL | |
| | FFAMLMVDKAGKNAFTHFNQQYRQSCNTPGFVPSEHALLDLLSASF | |
| | AAAGEQIDVTPFVELTGGVITQTQRDLNAFSHAKAVYPLYQLVDAQ | |
| | SLPAVLAQLKLDSSLRLVDSSQLKASGLKGDVALSLDIDDFAQIYG | |
| | EDLVLMEGARYAHKVRIDAPTLALNALPIGVYTLRLPTGKDRKYRP | |
| | TAGYLTVKQGKSAVTLRFERKAASPIVSQEINLLGLSDAVFATVVV | |
| | DRAKGAVVVDVTSTTPHAYFPGETYARVTLRDRQGAVRFSKEVQGT | |
| | QATLSHDELPFAEGDDIEIYHVEPSRVRLNPAAAGVIDNKNKTNVL | |
| | RIETSGLKNPALSGDPEQALLMRLEQAAQRLRRLLPAYYAPSSSFK | |
| | DDIYLAIGAFAEPQREPLLATYRDCLPADNTPPGANVGNAFTVAGK | |
| | GIGDRQFLTCAVDLVKKTVTVTLAAGIAHHYFADSYAFLSVTDADG | |
| | NTLLSYDVIGSQNQQAKTWVLPLSGYGGEVIHLRHEEPDNRLTIVN | |
| | EMQHVRLAEKGKQQAYCITPVGLKRIKD | |
| 24 | QVIQFNNETPVNNLQGDFEAMVIYAQNITVPHYNDKGDPRPHLTAL | VIBHAR2194 |
| | RDTLLLVKPVEPLNVSSQVKVIARDSQGNQLGALSLNTPEQLPNHD | from Vibrio |
| | GPNLDIMYAKDMVVSVTLPATWIQPGLTLELLNGSTSGLLSDLDIG | harveyi |
| | APNELLINTMDLGMLTNPRGRFYFADNPELHKDYFQKIPVSKLIVN | |
| | PYETVKLDEVMLPDGDLLTELDPSTGTVVHQGDMRADTTKILMSHG | |
| | INLANYGINSSTSVSERAHPYTANQITAIAAVGRYQNGVVAHGGSG | |
| | GNGMVTIDSSVGNEWSHEVGHNFGLGHWPGGTDGTTHRPSTDINSA | |
| | WGWDLFQKRFIANFMWNKRNGEDQVCCSDGIGIPAEEGYKFNRDAM | |
| | GGGEPTSPISKYTLHTPYVLEKIQTFMEGKATFDESSPTGFTKWND | |
| | ETKAMQAFQQPAILLSKSITSQSQLNTIKDDIDGSVLLGYINDFDI | |
| | TKVETGDGRWIRDIYLPSAENVAVGKAVNIARYSGYGVTAHVNGQL | |
| | VNLNRGDSKFYISDGKAWLETTEDQVAEGKPTRIPTDYGVPVTTLV | |
| | GYYDPQQQLDSYIFPALHGAYGFVYQPTPVDRLDTTGCYVKVYNGQ | |
| | DHQTDNYQLVGFRFDDNVMNKFH1NLKQTDDPTRAEVVCDNAVLSS | |
| | LDIEKPKQALEVSIVQSDSLKPSENNKPVAHAGEDQSVLSGATITL | |
| | SADKSTDADGDKLTYVWEQISGLPASIQASNAMTTNVVLAESTQEQ | |
| | SYVFSVLVSDGKSSSSDMVTIIAQPQPTQNHAPEVSLPSSIDAKPG | |
| | EVIEITATASDPDGDVLSFKWSTSGLAYQTMSGGTIQLSVPDVTVD | |
| | SQFLLSVVVSDPKGESASANTTLNVKASGNTCSVSDPNATNYDAWS | |
| | ASKSYSGGDLVSYKQLVWKAKHWSQNNQPDSSDAWELLSDVVLPWS | |
| | SQAAYSGGAQVTYNGVKEEAKWWTRGEQPNVSSVWINKGAACQ | |

What is claimed is:

1. A method comprising:
contacting a sample containing or suspected of containing a mucin-domain glycoprotein comprising a mucin-specific glycan-peptide cleavage motif with a mucin-specific protease that cleaves the cleavage sequence to generate glycopeptides and de-mucinated byproduct; and
analyzing the generated glycopeptides, the de-mucinated byproduct, or both.

2. The method according to claim 1, wherein the mucin-specific glycan-peptide cleavage motif comprises: S/T*-X-S/T, S/T*-S/T, X-S/T*, S/T*-S/T*, S/T*-X-X-X-X, wherein * denotes glycosylation of the S or T residue and X is any amino acid residue.

3. The method according to any of the preceding claims, wherein the method comprises detecting the presence of the mucin-domain glycoprotein in the sample based on detecting the generated glycopeptides.

4. The method according to any of the preceding claims, wherein the de-mucinated byproduct comprises de-mucinated cells.

5. The method according to claim 4, wherein the analyzing comprises evaluating a phenotype of the de-mucinated cells.

6. The method according to claim 4 or 5, further comprising comparing the de-mucinated cells to a control population of cells that are not de-mucinated.

7. The method according to any of the preceding claims, wherein the mucin-specific protease is a secreted protease of C1 esterase inhibitor (StcE) having at least 90% sequence identity with SEQ ID NO:1.

8. The method according to any one of claims 1-6, wherein the mucin-specific protease is selected from the group consisting of Pic, ZmpC, BT4244, AM0627, AM0908, AM1514, SmEnhancin, and VIBHAR2194.

9. The method according to any one of claims 1-6, wherein the mucin-specific protease is AM0627 or BT4244.

10. The method according to any of the preceding claims, wherein the sample is an acellular proteinaceous sample or a cellular sample.

11. The method according to claim 10, wherein the cellular sample is prepared from a cell culture or a biopsy.

12. The method according to claim 11, wherein the cell culture comprises cultured cancer cells.

13. The method according to claim 11, wherein the biopsy is a cancer biopsy.

14. The method according to any of the preceding claims, wherein the method further comprises enriching the sample for glycopeptides or isolating glycopeptides from the sample.

15. The method according to claim 14, wherein sample is enriched for the generated glycopeptides or the generated glycopeptides are isolated prior to the analyzing.

16. The method according to any of the preceding claims, wherein the analyzing comprises mass spectrometry.

17. The method according to any of the preceding claims, wherein the method further comprises determining the amino acid sequence of at least a portion of a glycopeptide of the generated glycopeptides.

18. The method according to claim 17, wherein the method further comprises identifying one or more glycosites of the glycopeptide.

19. The method according to any of the preceding claims, wherein the method does not comprise releasing glycans from the generated glycopeptides.

20. A method comprising:
contacting a cellular sample with a mucin-specific protease to generate a population of mucin-domain cleaved glycopeptides; and
analyzing the population of mucin-domain cleaved glycopeptides using mass spectrometry to produce a mucin-domain cleaved glycosignature.

21. The method according to claim 20, wherein the mucin-specific protease is a secreted protease of C1 esterase inhibitor (StcE) having at least 90% sequence identity with SEQ ID NO:1

22. The method according to claim 20, wherein the mucin-specific protease is selected from the group consisting of Pic, ZmpC, BT4244, AM0627, AM0908, AM1514, SmEnhancin, and VIBHAR2194.

23. The method according to claim 20, wherein the mucin-specific protease is AM0627 or BT4244.

24. The method according to any of claims 20 to 23, wherein the method further comprises isolating the population of cleaved glycopeptides prior to the analyzing.

25. The method according to any of claims 20 to 24, wherein the method further comprises analyzing a population of de-mucinated cells generated during the contacting.

26. The method according to claim 25, wherein the method further comprises isolating the population of cells prior to the analyzing.

27. The method according to any of claims 20 to 26, wherein the method further comprises deglycosylating glycoproteins of the cellular sample.

28. The method according to any of claims 20 to 27, wherein the method further comprises analyzing a population of deglycosylated glycoproteins using mass spectrometry.

29. The method according to any of claims 20 to 28, wherein the method further comprises analyzing a population of glycopeptides from the cellular sample using mass spectrometry to produce an intact glycosignature.

30. The method according to claim 29, wherein the method further comprises comparing the mucin-cleaved glycosignature to the intact glycosignature.

31. A method for detecting a condition characterized by aberrant glycosylation in a subject, the method comprising:
determining a mucin-domain cleaved glycosignature from a biological sample from said subject according to the method of any of claims 20 to 30; and
comparing the mucin-domain cleaved glycosignature to a healthy reference or control mucin-domain cleaved glycosignature to detect the condition.

32. The method according to claim 31, wherein the condition is cancer.

33. A method of treating a subject for a cancer, the method comprising:
performing, or having performed, the method according to claim 32 to detect whether a subject has a cancer characterized by aberrant glycosylation; and
treating the subject with a mucin-domain directed therapy when the subject is identified as having the cancer characterized by aberrant glycosylation.

34. The method according to claim 33, wherein the mucin-domain directed therapy comprises a mucin-domain glycoprotein-specific antibody.

35. The method according to claim 33 or 34, wherein the mucin-domain directed therapy comprises a mucin-domain glycoprotein-specific chimeric antigen receptor (CAR).

36. The method according to any of claims 33 to 35, wherein the mucin-domain directed therapy comprises an anti-mucin vaccine.

37. The method according to any of claims 33 to 36, wherein the mucin-domain directed therapy comprises a mucin inhibitor.

38. A method of identifying a receptor as mucin-domain glycoprotein-specific, the method comprising:
contacting a cellular sample with a mucin-specific protease to generate a de-mucinated cellular sample; and
assessing binding of the receptor with the cellular sample and the de-mucinated cellular sample, wherein decreased binding of the receptor to cells of the de-mucinated cellular sample as compared to cells of the cellular sample identifies the receptor as a mucin-domain glycoprotein-specific receptor.

39. The method according to claim 38, wherein the mucin-specific protease is a secreted protease of C1 esterase inhibitor (StcE) having at least 90% sequence identity with SEQ ID NO:1

40. The method according to claim 39, wherein the mucin-specific protease is selected from the group consisting of Pic, ZmpC, BT4244, AM0627, AM0908, AM1514, SmEnhancin, and VIBHAR2194.

41. The method according to claim 40, wherein the mucin-specific protease is AM0627 or BT4244.

42. The method according to any of claims 38 to 41, wherein the receptor is an orphan receptor.

43. The method according to claims 38 to 42, wherein the method further comprises assessing binding of a control receptor known to be mucin-domain glycoprotein-specific.

44. The method according to any of claims 38 to 43, wherein the method further comprises assessing binding of a control receptor known not to be mucin-domain glycoprotein-specific.

45. A method comprising:
contacting a sample with a catalytically inactive mucin-specific protease that binds a mucin-domain glycoprotein present in the sample; and
separating the bound mucin-specific protease from at least a portion of the sample to isolate, enrich or deplete the mucin-domain glycoprotein from or in the sample.

46. The method according to claim 45, wherein the catalytically inactive mucin-specific protease is: a mutant that lacks protease activity, in the presence of a protease inhibitor, or both.

47. The method according to claim 45 or 46, wherein the mucin-specific protease is a secreted protease of C1 esterase inhibitor (StcE) having at least 90% sequence identity with SEQ ID NO:1

48. The method according to claim 47, wherein the StcE has 100% sequence identity with SEQ ID NO:1.

49. The method according to claim 47, wherein the StcE is a recombinant StcE variant having less than 100% sequence identity with SEQ ID NO:1.

50. The method according to claim 49, wherein recombinant StcE variant comprises a E447D mutation.

51. The method according to claim 45 or 46, wherein the mucin-specific protease is selected from the group consisting of Pic, ZmpC, BT4244, AM0627, AM0908, AM1514, SmEnhancin, and VIBHAR2194.

52. The method according to claim 45 or 46, wherein the mucin-specific protease is BT4244 or AM0627.

53. The method according to claim 52, wherein the mucin-specific protease is a recombinant AM0627 variant comprising a substitution at amino acid position 326 or a recombinant BT4244 variant comprising a substitution at amino acid position 575.

54. The method according to claim 53, wherein the substitution at amino acid position E326 is E326A.

55. The method according to claim 53, wherein the substitution at amino acid position E575 is E575A.

56. The method according to any one of claims 45 to 55, wherein the mucin-specific protease is bound to a solid support.

57. The method according to claim 56, wherein the method comprises contacting the sample with the solid support to bind the mucin-domain glycoprotein and extracting the solid support from the sample to isolate the mucin-domain glycoprotein.

58. The method according to claim 56, wherein the method comprises contacting the sample with the solid support to bind the mucin-domain glycoprotein and retaining the solid support to enrich the sample for the mucin-domain glycoprotein.

59. The method according to any of claims 45 to 58, wherein the mucin-domain glycoprotein isolated, enriched, or depleted is an intact mucin-domain glycoprotein.

60. A kit comprising:
one or more containers comprising a mucin-specific protease.

61. The kit according to claim 60, wherein the mucin-specific protease is a secreted protease of C1 esterase inhibitor (StcE) having at least 90% sequence identity with SEQ ID NO:1 or a nucleic acid encoding the StcE.

62. The kit according to claim 60, wherein the StcE is a recombinant StcE variant having less than 100% sequence identity with SEQ ID NO:1.

63. The kit according to claim 60, wherein the mucin-specific protease is selected from the group consisting of Pic, ZmpC, BT4244, AM0627, AM0908, AM1514, SmEnhancin, and VIBHAR2194.

64. The kit according to claim 60, wherein the mucin-specific protease is AM0627 or VIBHAR2194.

65. The kit according to any one of claims 60-64, wherein the mucin-specific protease is conjugated to a detectable label.

66. The kit according to claim 65, wherein the detectable label comprises a fluorescent molecule, luminescent molecule, light-scattering molecule, or a quantum dot.

67. The kit according to any of claims 60 to 66, wherein the mucin-specific protease is catalytically inactive, the kit comprises a protease inhibitor, or both.

68. The kit according to any of claims 60 to 66, wherein the kit comprises the mucin-specific protease in a dry composition.

69. The kit according to claim 68, wherein the mucin-specific protease is lyophilized.

70. The kit according to any of any of claims 60 to 69, wherein the mucin-specific protease is attached to a solid support.

71. The kit according to any of claims 60 to 61, wherein the kit comprises a plasmid comprising a nucleic acid encoding the mucin-specific protease.

72. The kit according to any of claims 60 to 70, further comprising a buffer in which the mucin-specific protease is active.

73. The kit according to any of claims 60 to 72, further comprising a deglycosylase.

74. The kit according to claim 73, wherein the deglycosylase is PNGase F.

75. The kit according to any of claims 60 to 74, further comprising a protease.

76. The kit according to claim 75, wherein the protease is trypsin.

77. The kit according to any of claims 60 to 76, further comprising one or more purification devices and/or reagents.

78. A method comprising:
contacting a sample with a catalytically inactive mucin-specific protease that binds a mucin-domain glycoprotein present in the sample;
detecting binding of the catalytically inactive mucin-specific protease to the sample.

79. The method according to claim 78, wherein the catalytically inactive mucin-specific protease is a variant of a mucin-specific protease selected from the group consisting of StcE, Pic, ZmpC, BT4244, AM0627, AM0908, AM1514, SmEnhancin, and VIBHAR2194.

80. The method according to claim 78, wherein the mucin-specific protease is StcE, BT4244, or AM0627.

81. The method according to claim 80, wherein the catalytically inactive mucin-specific protease comprises a sequence of StcE comprising the substitution E447D.

82. The method according to claim 80, wherein the catalytically inactive mucin-specific protease comprises a sequence of BT4244 comprising the substitution E575A.

83. The method according to claim 80, wherein the catalytically inactive mucin-specific protease comprises a sequence of AM0627 comprising the substitution E326A.

84. The method according to any of claims 78-83, wherein the sample is a tissue sample.

85. The method according to claim 84, wherein the tissue sample is a small intestinal tissue sample.

86. The method according to any of claims 80-85, wherein the catalytically inactive mucin-specific protease comprises a detectable label.

87. The method according to claim 86, wherein the detectable label is a fluorescent molecule, luminescent molecule, light-scattering molecule, a quantum dot, or an affinity label, such as biotin.


SEQUENCE LISTING (25 sequences)

SEQ ID NO: 1 - protein, 863 aa, Escherichia coli
ADNNSAIYFNTSQPINDLQGSLAAEVKFAQSQILPAHPKEGDSQPHLTSLRKSLLLVRPVKADDKTPVQVEARDDNNKILGTLTLYPPSSLPDTIYHLDGVPEGGIDFTPHNGTKKIINTVAEVNKLSDASGSSIHSHLTNNALVEIHTANGRWVRDIYLPQGPDLEGKMVRFVSSAGYSSTVFYGDRKVTLSVGNTLLFKYVNGQWFRSGELENNRITYAQHIWSAELPAHWIVPGLNLVIKQGNLSGRLNDIKIGAPGELLLHTIDIGMLTTPRDRFDFAKDKEAHREYFQTIPVSRMIVNNYAPLHLKEVMLPTGELLTDMDPGNGGWHSGTMRQRIGKELVSHGIDNANYGLNSTAGLGENSHPYVVAQLAAHNSRGNYANGIQVHGGSGGGGIVTLDSTLGNEFSHEVGHNYGLGHYVDGFKGSVHRSAENNNSTWGWDGDKKRFIPNFYPSQTNEKSCLNNQCQEPFDGHKFGFDAMAGGSPFSAANRFTMYTPNSSAIIQRFFENKAVFDSRSSTGFSKWNADTQEMEPYEHTIDRAEQITASVNELSESKMAELMAEYAVVKVHMWNGNWTRNIYIPTASADNRGSILTINHEAGYNSYLFINGDEKVVSQGYKKSFVSDGQFWKERDVVDTREARKPEQFGVPVTTLVGYYDPEGTLSSYIYPAMYGAYGFTYSDDSQNLSDNDCQLQVDTKEGQLRFRLANHRANNTVMNKFHINVPTESQPTQATLVCNNKILDTKSLTPAPEGLTYTVNGQALPAKENEGCIVSVNSGKRYCLPVGQRSGYSLPDWIVGQEVYVDSGAKAKVLLSDWDNLSYNRIGEFVGNVNPADMKKVKAWNGQYLDFSKPRSMRVVYK

SEQ ID NO: 2 - DNA, 2697 bp, Escherichia coli
atgaacactaaaatgaatgagagatggagaacaccgatgaaattaaagtatctgtcatgtacgatccttgcccctctggcgattggggtattttctgcaacagctgctgataataattcagccatttatttcaatacctcccagcctataaatgatctgcagggttcgttggccgcagaggtgaaatttgcacaaagccagattttacccgcccatcctaaagaaggggatagtcaaccacatctgaccagcctgcggaaaagtctgctgcttgtccgtccggtgaaagctgatgataaaacacctgttcaggtggaagcccgcgatgataataataaaattctcggtacgttaaccctttatcctccttcatcactaccggatacaatctaccatctggatggtgttccggaaggtggtatcgatttcacacctcataatggaacgaaaaagatcattaatacggtggctgaagtaaacaaactcagtgatgccagcgggagttctattcatagccatctaacaaataatgcactggtggagatccatactgcaaatggtcgttgggtaagagacatttatctgccgcagggacccgaccttgaaggtaagatggttcgctttgtttcgtctgcaggctatagttcaacggttttttatggtgatcgaaaagtcacactctcggtgggtaacactcttctgttcaaatatgtaaatggtcagtggttccgctccggtgaactggagaataatcgaatcacttatgctcagcatatttggagtgctgaactgcctgcgcactggatcgtgcctggtttaaacttggtgattaaacagggcaatctgagcggtcgcctaaatgatatcaagattggagcaccgggtgagctgttgttgcatacaattgatatcgggatgttgaccactccccgggatcgctttgattttgccaaagacaaagaagcacatagggaatatttccagaccattcctgtaagtcgtatgattgttaataattatgcgcctctacacctaaaggaagttatgttaccaaccggagagttattgacagatatggatccaggaaatggtgggtggcatagtggtacaatgcgtcaaagaataggtaaagaattggtttcgcatggcattgataatgctaactatggtttaaatagtaccgcaggcttaggggagaatagtcatccatatgtagttgcgcaattagcggcacataatagccgcggtaattatgctaatggcatccaggttcatggtggctccggaggtgggggaattgttactttagattccacattggggaatgagttcagtcatgaagttggtcataattatggtcttggtcattatgtagatggtttcaagggttctgtacatcgtagtgcagaaaataacaactcaacttggggatgggatggtgataaaaaacggtttattcctaacttttatccgtctcaaacaaatgaaaagagttgtctgaataatcagtgtcaagaaccgtttgatggacacaaatttggttttgacgccatggcgggaggcagccctttctctgctgcaaaccgtttcacaatgtatactccgaattcatcggctatcatccagcgtttttttgaaaataaagctgtgttcgatagccgttcctccaccggcttcagcaagtggaatgcagatacgcaggaaatggaaccgtatgaacacaccattgaccgtgcggagcagattacggcttcagtcaatgagctaagtgaaagcaaaatggctgagctgatggcagagtacgctgtcgtcaaagtgcatatgtggaacggtaactggacaagaaacatctatatccctacagcctccgcagataatagaggcagtatcctgaccatcaaccatgaggccggttataatagttatctgtttataaatggtgacgaaaaggtcgtttcccaggggtataaaaagagctttgtttccgatggtcagttctggaaagaacgtgatgtggttgatactcgtgaagcgcgtaagccagagcagtttggtgttcctgtgacgaccctggtggggtattacgatccggaaggcacgctgtcaagctacatctatcctgcgatgtatggtgcctatggcttcacttattccgatgatagtcagaatctatccgataacgactgccagctgcaggtggatacgaaagaagggcagttgcgattcagactggctaatcaccgggctaacaacactgtaatgaataagttccatattaacgtgccaacagaaagtcagcccacacaggccacattggtttgcaataacaagatactggataccaaatcgctcacacctgcgccagaaggacttacctatactgtaaatgggcaggcacttccagcaaaagaaaacgagggatgcatcgtgtccgtgaattcaggtaaacgttactgtttgccggttggtcaacggtcaggatatagccttcctgactggattgttgggcaggaagtctatgtcgacagcggggctaaagcgaaagtgctgctttctgactgggataacctgtcctataacaggattggtgagtttgtaggtaatgtgaacccagctgatatgaaaaaagttaaagcctggaacggacagtatttggacttcagtaaacctaggtcaatgagggttgtatataaataa

SEQ ID NO: 3 - protein, 9 aa, Artificial sequence
  synthetic sequence
  CARBOHYD (5)..(5)
RPPITQSSL

SEQ ID NO: 4 - protein, 6 aa, Artificial sequence
  synthetic sequence
  MOD_RES (1)..(1): ACETYLATION
  CARBOHYD (1)..(1): The Amino Acid at position 1 is modified with GalNAcalpha
  CARBOHYD (3)..(3): The Amino Acid at position 3 is modified with GalNAcalpha
  MOD_RES (6)..(6): METHYLATION
PTLTHN

SEQ ID NO: 5 - protein, 9 aa, Artificial sequence
  synthetic sequence
LSTMMSPTT

SEQ ID NO: 6 - protein, 10 aa, Artificial sequence
  synthetic sequence
STNASTVPFR

SEQ ID NO: 7 - protein, 6 aa, Artificial sequence
  synthetic sequence
  MOD_RES (1)..(1): ACETYLATION
  MOD_RES (6)..(6): METHYLATION
PTLTHN

SEQ ID NO: 8 - protein, 6 aa, Artificial sequence
  synthetic sequence
  MOD_RES (1)..(1): ACETYLATION
  CARBOHYD (1)..(1): The amino acid at position 1 is modified with GalNAcalpha
  MOD_RES (6)..(6): METHYLATION
PTLTHN

SEQ ID NO: 9 - DNA, 27 bp, Artificial sequence
  synthetic sequence
tcagtcatgacgttggtcataattatg

SEQ ID NO: 10 - DNA, 18 bp, Artificial sequence
  synthetic sequence
actcattccccaatgtgg

SEQ ID NO: 11 - protein, 9 aa, Artificial sequence
  synthetic sequence
  CARBOHYD (5)..(5): The amino acid at position 5 is modified with GalNAc
RPPITQSSL

SEQ ID NO: 12 - protein, 9 aa, Artificial sequence
  synthetic sequence
  CARBOHYD (4)..(4): The amino acid at position 4 is modified with GalNAc
IPVSSHNSL

SEQ ID NO: 13 - protein, 5 aa, Artificial sequence
  synthetic sequence
SHNSL

SEQ ID NO: 14 - protein, 8 aa, Artificial sequence
  synthetic sequence
  MOD_RES (4)..(4): PHOSPHORYLATION
DRVYIHPF

SEQ ID NO: 15 - protein, 6 aa, Artificial sequence
  synthetic sequence
  CARBOHYD (5)..(5)
RPPITQ

SEQ ID NO: 16 - protein, 7 aa, Artificial sequence
  synthetic sequence
  CARBOHYD (3)..(3)
GPTPSAA

SEQ ID NO: 17 - protein, 1372 aa, Escherichia coli
MNKVYSLKYCPVTGGLIAVSELARRVIKKTCRRLTHILLAGIPAICLCYSQISQAGIVRSDIAYQIYRDFAENKGLFVPGANDIPVYDKDGKLVGRLGKAPMADFSSVSSNGVATLVSPQYIVSVKHNGGYRSVSFGNGKNTYSLVDRNNHPSIDFHAPRLNKLVTEVIPSAVTSEGTKANAYKYTERYTAFYRVGSGTQYTKDKDGNLVKVAGGYAFKTGGTTGVPLISDATIVSNPGQTYNPVNGPLPDYGAPGDSGSPLFAYDKQQKKWVIVAVLRAYAGINGATNWWNVIPTDYLNQVMQDDFDAPVDFVSGLGPLNWTYDKTSGTGTLSQGSKNWTMHGQKDNDLNAGKNLVFSGQNGAIILKDSVTQGAGYLEFKDSYTVSAESGKTWTGAGIITDKGTNVTWKVNGVAGDNLHKLGEGTLTINGTGVNPGGLKTGDGIVVLNQQADTAGNIQAFSSVNLASGRPTVVLGDARQVNPDNISWGYRGGKLDLNGNAVTFTRLQAADYGAVITNNAQQKSQLLLDLKAQDTNVSEPTIGNISPFGGTGTPGNLYSMILNSQTRFYILKSASYGNTLWGNSLNDPAQWEFVGMDKNKAVQTVKDRILAGRAKQPVIFHGQLTGNMDVAIPQVPGGRKVIFDGSVNLPEGTLSQDSGTLIFQGHPVIHASISGSAPVSLNQKDWENRQFTMKTLSLKDADFHLSRNASLNSDIKSDNSHITLGSDRAFVDKNDGTGNYVIPEEGTSVPDTVNDRSQYEGNITLNHNSALDIGSRFTGGIDAYDSAVSITSPDVLLTAPGAFAGSSLTVHDGGHLTALNGLFSDGHIQAGKNGKITLSGTPVKDTANQYAPAVYLTDGYDLTGDNAALEITRGAHASGDIHASAASTVTIGSDTPAELASAETAASAFAGSLLEGYNAAFNGAITGGRADVSMHNALWTLGGDSAIHSLTVRNSRISSEGDRTFRTLTVNKLDATGSDFVLRTDLKNADKINVTEKATGSDNSLNVSFMNNPAQGQALNIPLVTAPAGTSAEMFKAGTRVTGFSRVTPTLHVDTSGGNTKWILDGFKAEADKAAAAKADSFMNAGYKNFMTEVNNLNKRMGDLRDTNGDAGAWARIMSGAGSADGGYSDNYTHVQVGFDKKHELDGVDLFTGVTMTYTDSSADSHAFSGKTKSVGGGLYASALFESGAYIDLIGKYIHHDNDYTGNFASLGTKHYNTHSWYAGAETGYRYHLTEDTFIEPQAELVYGAVSGKTFRWKDGDMDLSMKNRDFSPLVGRTGVELGKTFSGKDWSVTARAGTSWQFDLLNNGETVLRDASGEKRIKGEKDSRMLFNVGMNAQIKDNMRFGLEFEKSAFGKYNVDNAVNANFRYMF

SEQ ID NO: 18 - protein, 1704 aa, Streptococcus pneumoniae
LSAYNSQLSIGVGEHLPEPLKIEGYQYIGYIKTKKQDNTELSRTVDGKYSAQRDSQPNSTKTSDVVHSADLEWNQGQGKVSLQGEASGDDGLSEKSSIAADNLSSNDSFASQVEQNPDHKGESVVRPTVPEQGNPVSATTVQSAEEEVLATTNDRPEYKLPLETKGTQEPGHEGEAAVREDLPVYTKPLETKGTQGPGHEGEAAVREEEPAYTEPLATKGTQEPGHEGKATVREETLEYTEPVATKGTQEPEHEGEAAVEEELPALEVTTRNRTEIQNIPYTTEEIQDPTLLKNRRKIERQGQAGTRTIQYEDYIVNGNVVETKEVSRTEVAPVNEVVKVGTLVKVKPTVEITNLTKVENKKSITVSYNLIDTTSAYVSAKTQVFHGDKLVKEVDIENPAKEQVISGLDYYTPYTVKTHLTYNLGENNEENTETSTQDFQLEYKKIEIKDIDSVELYGKENDRYRRYLSLSEAPTDTAKYFVKVKSDRFKEMYLPVKSITENTDGTYKVTVAVDQLVEEGTDGYKDDYTFTVAKSKAEQPGVYTSFKQLVTAMQSNLSGVYTLASDMTADEVSLGDKQTSYLTGAFTGSLIGSDGTKSYAIYDLKKPLFDTLNGATVRDLDIKTVSADSKENVAALAKAANSANINNVAVEGKISGAKSVAGLVASATNTVIENSSFTGKLIANHQDSNKNDTGGIVGNITGNSSRVNKVRVDALISTNARNNNQTAGGIVGRLENGALISNSVATGEIRNGQGYSRVGGIVGSTWQNGRVNNVVSNVDVGDGYVITGDQYAAADVKNASTSVDNRKADRFATKLSKDQIDAKVADYGITVTLDDTGQDLKRNLREVDYTRLNKAEAERKVAYSNIEKLMPFYNKDLVVHYGNKVATTDKLYTTELLDVVPMKDDEVVTDINNKKNSINKVMLHFKDNTVEYLDVTFKENFINSQVIEYNVTGKEYIFTPEAFVSDYTAITNNVLSDLQNVTLNSEATKKVLGAANDAALDNLYLDRQFEEVKANIAEHLRKVLAMDKSINTTGDGVVEYVSEKIKNNKEAFMLGLTYMNRWYDINYGKMNTKDLSTYKFDFNGNNETSTLDTIVALGNSGLDNLRASNTVGLYANKLASVKGEDSVFDFVEAYRKLFLPNKTNNEWFKENTKAYIVEMKSDIAEVREKQESPTADRKYSLGVYDRISAPSWGHKSMLLPLLTLPEESVYISSNMSTLAFGSYERYRDSVDGVILSGDALRTYVRNRVDIAAKRHRDHYDIWYNLLDSASKEKLFRSVIVYDGFNVKDETGRTYWARLTDKNIGSIKEFFGPVGKWYEYNSSAGAYANGSLTHFVLDRLLDAYGTSVYTHEMVHNSDSAIYFEGNGRREGLGAELYALGLLQSVDSVNSHILALNTLYKAEKDDLNRLHTYNPVERFDSDEALQSYMHGSYDVMYTLDAMEAKAILAQNNDVKKKWFRKIENYYVRDTRHNKDTHAGNKVRPLTDEEVANLTSLNSLIDNDIINRRSYDDSREYKRNGYYTISMFSPVYAALSNSKGAPGDIMFRKIAYELLAEKGYHKGFLPYVSNQYGAEAFASGSKTFSSWHGRDVALVTDDLVFKKVFNGEYSSWADFKKAMFKQRIDKQDNLKPITIQYELGNPNSTKEVTITTAAQMQQLINEAAAKDITNIDRATSHTPASWVHLLKQKIYNAYLRTTDDFRNSIYK

SEQ ID NO: 19 - protein, 834 aa, Bacteroides thetaiotaomicron
KDTEKSIINSSFSISEEYLIQNLDKSSTSVQIPINTSMELAQWSVSYEANWLQCSKQKTAAEGTFLRITVNENTGETKRTANIKVTSTTATYTITVNQYAKGEVIVEGDIKVTPTGGKASEHQEGQDIENTYDGKFSTDGAAPFHTPWGQSAKFPVTLEYYFKGDTEIDYLIYYTRSGNGNFGKVKVYTTTNPDRSDYTLQGEYDFKEQNAPSKVSFSEGIKATGIKFEVLSGLGDFVSCDEMEFYKTNTDKTLDKQLLTVFTDITCTEIKNNVTNEQIQALPDYFVRIAEAVRDNTYDKWEKEFRIRSYEPYSNIAEWADKLMTKKYSDLDNPTGISVKAGDDIIVLVGDTYGQNISMQCIWETGTEYKQTASSGDVYMLNPGVNKLTMKGEGQLFVMYNTELTSNTAKPIKIHIPLGSGTVNGFFDLKEHKTDEKYAELLKKSTHKYFCIRGEKIMFYFHRNKLLEYVPNNILSAIHLWDNIVGWQQELMGIDDVRPSQVNNHLFAISPEGSYMWASDYQIGFVYTYLGNILLEDNVMAAEDNAWGPAHEIGHVHQAAINWASSTESSNNLFSNFIIYKLGKYKSRGNGLGSVATARYANGQAWYNMGDATHQNEDTETHMRMNWQLWIYYHRCEYKTDFWQTLFKLMREVNMTEGEDPGKKQLEFAKMASKAANQNLTDFFEMWGFFEPVNTTIEQYGTYKYYVSDAMIREAKEYMAQFPAPKHAFQYIEDRKKSEFPSNDYRYSAVGDVGYYTQFKENQKITKAITAELAGRKVSIQNGDEAVAFELRENDENGKLLYFSTFTTFEIPSSILMVNAKLYAVQADGKRILL

SEQ ID NO: 20 - protein, 486 aa, Akkermansia muciniphila
ANTPEHIGNDLKLFKDSSCTSLKPDVKNTSAFQSDAMKELATKILAGHYKPDYLYAEYRALPSPRQTGKNLRIGDGFSKYDNMTGVYLEKGRHVVLVGKTEGQEISLLLPNLMRKPAEGVQPTKDPNGWGLHKKQIPLKEGINIIDVETPANAYISYFTEDAGKAPKIPVHFVTGKANGYFDTTRGDTNKDWVRLLDQAVSPIMDARGKYIQVAYPVEFLKKFTKDRGTELINAYDKLIGIQYQLMGLDKYGKIPENRVLARVNFNYYMFRDGDGVAYLGNDGTMRMVTDPENVLKGDACWGFSHEVGHVMQMRPMTWGGMTEVSNNIFSLQAAAKTGNESRLKRQGSYDKARKEIIEGEIAYLQSKDVFNKLVPLWQLHLYFTKNGHPDFYPDVMEYLRNNAGNYGGNDTVKYQFEFVKACCDVTKTDLTDFFEKWGFFKPGKFHIGDYAQYDFNVTPEMVEETKKWIAGKGYPKPETDITELSE

SEQ ID NO: 21 - protein, 720 aa, Akkermansia muciniphila
KYPSLDVPEDILLHVKEAASSQSPYSGNHSVKQAVDGSMESANWHVPGVHHVEGEFVFEVPETIHYIIFSGANFNEIAVSAMSGSSWKDLGKFDIGGSRMIRFKKPLQKVRKIRLTVDYPEGSSPSFTVREISFYKRVESALNRKLLKVFKDTSCSSINPRCTLTDLKALPEFLQMIAKKIKSGDYEDKEFRIASYKAYSHPEFAAKVRNINALNKFDNPTGIVAEKGDEILVFVGPTHGEDIGLASVSPAGIESSSYPLNEGVNKIRINRSGLLYVMYHTDISPPKKPITVHIPVGSGIVNGYFDVTRHTDKDWKRMISNAPHSMFDIVGRNSMMILHTKYLKDYSPDSITKSVRVWDESVKAMWKIMGFDKYPQPHNNRQLGVSVEGGAHMFATWYYCGYSIGDQGNTLKNEVLAPGVLQGNRLWGIGHEIGHCYQHPFNWRSMSESSNNFFAQLILDQVTNAINGNEQASDMENPCKYLLSEAVKGMPFHDLNGWAKWGFAQYSFYLYFHKLGINPEFYPRLFESLRRKPLSRQAYEVSEAHLALYERICNISRTDFTDDFEIFNWFVPIDRKGHQYGDYSFKMTEEMARASKARIAAKRYPKPKFRIAFLHQHGKTVNLWGQNLHGSQLNGYWTKYKQNAKLSPSVSASKKDNMIIVRNGENAAAFCVVTNGKVVGYYDRQKFDVSGVEWNDTSKVYAIPIQTAEPYKLIYAAGRS

SEQ ID NO: 22 - protein, 612 aa, Akkermansia muciniphila
QDNLAKRKAAQELTAERKLKEKKAREAAEKQRIKREREIREKKEKERLAAQKAYEEAQEEKARQAAEAARKLQEQAEREEREKRRREELERREREEEARRQEEDTPVEEEPEPEGRFPQPVKNRMPELSVYSIPCRDDIQTEKDKLLETWSWDKAEKMEGMEEFPTGSSPWKKGKDAGRMQALLEKCREWKDAKLASLKACPAAKDFPGVPENGAQTVRRTVEIDSNIGGWHSTGLYAPPGAEISCSLSGAPKDGSISVRIGCHTDSLHKLDEWKRVPEITMQVPAGRGRVKMVNPMGGLVYVNVGQRPRRGKVFKVQISGAVPSPLFVMGKTTPEQWAEQLENTKAPWGEIRMPRLIVTMPVEQLKQCPDVQKTAEFLQKNMALQDWIMGWDTKPDRLHHPMRFVVDRQISAGAGHSGYPAMATKDWTNSIATGSIIHSGSWGLWHELGHNHQSPPFTMEGQTEVSVNIFSMVCEVMGTGKDFESCWGGGMGPYGMSAEMKKYFSGTQTYNEAPNKVQLFFWVELMYYLGFDAFRQVALQFHDKPYDNGELSDEKKWEWVMNAFSKVTGKNMGPFFKIWRTPVSERATGRMKDLPAWLPSKDYPACYTAEE

SEQ ID NO: 23 - protein, 855 aa, Serratia marcescens
MPTITKTQSLYTLTRPVWLDAAGMSKGIDHDRQHLGIILAAGQVLRVRQTNAAFTGKLTLRLLNDDSKTEAEFSVGASWVEAQVNAVSVPFIDTPYGQGEPALEFEYPDTAKALPVYRRGENEAAFFARWDDRDAEFALVDAEYAVLLVPKISKPALKSLGEAKNIDGLIAYYDRIFTFYNALTGVSFEAEHESDRNIRNRYFMKADKHGAGAAYYGGRWTAETSSSVSSFWLTPKDSNWGSLHEIAHGYQGHFMGDKYFSTGEMWNNIYAACYQDVMLGERKYQEGWLYNYGNAAGVEKKIVDLIAQGTPLNQWDLRSKLFFAMLMVDKAGKNAFTHFNQQYRQSCNTPGFVPSEHALLDLLSASFAAAGEQIDVTPFVELTGGVITQTQRDLNAFSHAKAVYPLYQLVDAQSLPAVLAQLKLDSSLRLVDSSQLKASGLKGDVALSLDIDDFAQIYGEDLVLMEGARYAHKVRIDAPTLALNALPIGVYTLRLPTGKDRKYRPTAGYLTVKQGKSAVTLRFERKAASPIVSQEINLLGLSDAVFATVVVDRAKGAVVVDVTSTTPHAYFPGETYARVTLRDRQGAVRFSKEVQGTQATLSHDELPFAEGDDIEIYHVEPSRVRLNPAAAGVIDNKNKTNVLRIETSGLKNPALSGDPEQALLMRLEQAAQRLRRLLPAYYAPSSSFKDDIYLAIGAFAEPQREPLLATYRDCLPADNTPPGANVGNAFTVAGKGIGDRQFLTCAVDLVKKTVTVTLAAGIAHHYFADSYAFLSVTDADGNTLLSYDVIGSQNQQAKTWVLPLSGYGGEVIHLRHEEPDNRLTIVNEMQHVRLAEKGKQQAYCITPVGLKRIKD

SEQ ID NO: 24 - protein, 961 aa, Vibrio harvevi
QVIQFNNETPVNNLQGDFEAMVIYAQNITVPHYNDKGDPRPHLTALRDTLLLVKPVEPLNVSSQVKVIARDSQGNQLGALSLNTPEQLPNHDGPNLDIMYAKDMWSVTLPATWIQPGLTLELLNGSTSGLLSDLDIGAPNELLINTMDLGMLTNPRGRFYFADNPELHKDYFQKIPVSKLIVNPYETVKLDEVMLPDGDLLTELDPSTGTWHQGDMRADTTKILMSHGINLANYGINSSTSVSERAHPYTANQITAIAAVGRYQNGVVAHGGSGGNGMVTIDSSVGNEWSHEVGHNFGLGHWPGGTDGTTHRPSTDINSAWGWDLFQKRFIANFMWNKRNGEDQVCCSDGIGIPAFEGYKFNRDAMGGGEPTSPISKYTLHTPYVLEKIQTFMEGKATFDESSPTGFTKWNDETKAMQAFQQPAILLSKSITSQSQLNTIKDDIDGSVLLGYINDFDITKVETGDGRWIRDIYLPSAENVAVGKAVNIARYSGYGVTAHVNGQLVNLNRGDSKFYISDGKAWLETTEDQVAEGKPTRIPTDYGVPVTTLVGYYDPQQQLDSYIFPALHGAYGFVYQPTPVDRLDTTGCYVKVYNGQDHQTDNYQLVGFRFDDNVMNKFHINLKQTDDPTRAEVVCDNAVLSSLDIEKPKQALEVSIVQSDSLKPSENNKPVAHAGEDQSVLSGATITLSADKSTDADGDKLTYVWEQISGLPASIQASNAMTTNVVLAESTQEQSYVFSVLVSDGKSSSSDMVTIIAQPQPTQNHAPEVSLPSSIDAKPGEVIEITATASDPDGDVLSFKWSTSGLAYQTMSGGTIQLSVPDVTVDSQFLLSVVVSDPKGESASANTTLNVKASGNTCSVSDPNATNYDAWSASKSYSGGDLVSYKQLVWKAKHWSQNNQPDSSDAWELLSDVVLPWSSQAAYSGGAQVTYNGVKFEAKWWTRGEQPNVSSVWINKGAACQ

SEQ ID NO: 25 - protein, 5 aa, Artificial sequence
  synthetic sequence
  VARIANT (1)..(1): The amino acid at position 1 can be Ser or Thr
  CARBOHYD (1)..(1)
  VARIANT (2)..(5): The amino acids at positions 2 to 5 can be any amino acid
XXXXX